SEQUENCE LISTING (119 sequences)

SEQ ID NO: 1 - protein, 8 aa, Artificial Sequence
  source: /note=“Description of Artificial Sequence: Synthetic peptide”
GFTFSSYA

SEQ ID NO: 2 - protein, 8 aa, Artificial Sequence
  source: /note=“Description of Artificial Sequence: Synthetic peptide”
ISVSGGST

SEQ ID NO: 3 - protein, 10 aa, Artificial Sequence
  source: /note=“Description of Artificial Sequence: Synthetic peptide”
AKANWGFFDY

SEQ ID NO: 4 - protein, 9 aa, Artificial Sequence
  source: /note=“Description of Artificial Sequence: Synthetic peptide”
SSDVGGYNY

SEQ ID NO: 5 - protein, 3 aa, Artificial Sequence
  source: /note=“Description of Artificial Sequence: Synthetic peptide”
DVS

SEQ ID NO: 6 - protein, 8 aa, Artificial Sequence
  source: /note=“Description of Artificial Sequence: Synthetic peptide”
SSYADSVV

SEQ ID NO: 7 - protein, 25 aa, Artificial Sequence
  source: /note=“Description of Artificial Sequence: Synthetic peptide”
  VARIANT (6)..(6): /replace=“Glu”
  SITE (1)..(25): /note=“Variant residues given in the sequence have no preference with respect to those in the annotations for variant positions”
EVQLVQSGGGLVQPGGSLRLSCAAS

SEQ ID NO: 8 - protein, 17 aa, Artificial Sequence
  source: /note=“Description of Artificial Sequence: Synthetic peptide”
  VARIANT (1)..(1): /replace=“Leu”
  SITE (1)..(17): /note=“Variant residues given in the sequence have no preference with respect to those in the annotations for variant positions”
MSWVRQAPGKGLEWVSA

SEQ ID NO: 9 - protein, 38 aa, Artificial Sequence
  source: /note=“Description of Artificial Sequence: Synthetic polypeptide”
YYADSVKGRFTISRDNSKNTLYLQMNSLRAEDTAVYYC

SEQ ID NO: 10 - protein, 11 aa, Artificial Sequence
  source: /note=“Description of Artificial Sequence: Synthetic peptide”
WGQGTLVTVSS

SEQ ID NO: 11 - protein, 25 aa, Artificial Sequence
  source: /note=“Description of Artificial Sequence: Synthetic peptide”
  VARIANT (1)..(1): /replace=“Gln”
  VARIANT (2)..(2): /replace=“Ser”
  VARIANT (3)..(3): /replace=“Ala”
  SITE (1)..(25): /note=“Variant residues given in the sequence have no preference with respect to those in the annotations for variant positions”
SYELTQPRSVSGSPGQSVTISCTGT

SEQ ID NO: 12 - protein, 17 aa, Artificial Sequence
  source: /note=“Description of Artificial Sequence: Synthetic peptide”
  VARIANT (17)..(17): /replace=“Tyr”
  SITE (1)..(17): /note=“Variant residues given in the sequence have no preference with respect to those in the annotations for variant positions”
VSWYQQHPGKAPKLMIF

SEQ ID NO: 13 - protein, 36 aa, Artificial Sequence
  source: /note=“Description of Artificial Sequence: Synthetic polypeptide”
KRPSGVPDRFSGSKSGNTASLTISGLQAEDEADYYC

SEQ ID NO: 14 - protein, 10 aa, Artificial Sequence
  source: /note=“Description of Artificial Sequence: Synthetic peptide”
FGGGTKVTVL

SEQ ID NO: 15 - protein, 214 aa, Artificial Sequence
  source: /note=“Description of Artificial Sequence: Synthetic polypeptide”
QSALTQPRSVSGSPGQSVTISCTGTSSDVGGYNYVSWYQQHPGKAPKLMIFDVSKRPSGVPDRFSGSKSGNTASLTISGLQAEDEADYYCSSYADSVVFGGGTKVTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 16 - protein, 446 aa, Artificial Sequence
  source: /note=“Description of Artificial Sequence: Synthetic polypeptide”
EVQLVQSGGGLVQPGGSLRLSCAASGFTFSSYALSWVRQAPGKGLEWVSAISVSGGSTYYADSVKGRFTISRDNSKNTLYLQMNSLRAEDTAVYYCAKANWGFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 17 - protein, 214 aa, Artificial Sequence
  source: /note=“Description of Artificial Sequence: Synthetic polypeptide”
SYELTQPRSVSGSPGQSVTISCTGTSSDVGGYNYVSWYQQHPGKAPKLMIYDVSKRPSGVPDRFSGSKSGNTASLTISGLQAEDEADYYCSSYADSVVFGGGTKVTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 18 - protein, 446 aa, Artificial Sequence
  source: /note=“Description of Artificial Sequence: Synthetic polypeptide”
EVQLVESGGGLVQPGGSLRLSCAASGFTFSSYAMSWVRQAPGKGLEWVSAISVSGGSTYYADSVKGRFTISRDNSKNTLYLQMNSLRAEDTAVYYCAKANWGFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 19 - protein, 214 aa, Artificial Sequence; identical to SEQ ID NO: 15
  source: /note=“Description of Artificial Sequence: Synthetic polypeptide”

SEQ ID NO: 20 - protein, 446 aa, Artificial Sequence
  source: /note=“Description of Artificial Sequence: Synthetic polypeptide”
EVQLVESGGGLVQPGGSLRLSCAASGFTFSSYALSWVRQAPGKGLEWVSAISVSGGSTYYADSVKGRFTISRDNSKNTLYLQMNSLRAEDTAVYYCAKANWGFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 21 - protein, 214 aa, Artificial Sequence
  source: /note=“Description of Artificial Sequence: Synthetic polypeptide”
QSALTQPRSVSGSPGQSVTISCTGTSSDVGGYNYVSWYQQHPGKAPKLMIYDVSKRPSGVPDRFSGSKSGNTASLTISGLQAEDEADYYCSSYADSVVFGGGTKVTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 22 - protein, 445 aa, Artificial Sequence
  source: /note=“Description of Artificial Sequence: Synthetic polypeptide”
EVQLVESGGGLVQPGGSLRLSCAASGFTFSSYAMSWVRQAPGKGLEWVSAISVSGGSTYYADSVKGRFTISRDNSKNTLYLQMNSLRAEDTAVYYCAKANWGFFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVEPKSSDKTHTCPPCPAPPVAGPSVFLFPPKPKDTLMISRTPEVTCVVVDVSHEDPEVQFNWYVDGVEVHNAKTKPREEQAQSTFRVVSVLTVVHQDWLNGKEYKCAVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 23 - protein, 108 aa, Artificial Sequence
  source: /note=“Description of Artificial Sequence: Synthetic polypeptide”
QSALTQPRSVSGSPGQSVTISCTGTSSDVGGYNYVSWYQQHPGKAPKLMIYDVSKRPSGVPDRFSGSKSGNTASLTISGLQAEDEADYYCSSYADSVVFGGGTKVTVL

SEQ ID NO: 24 - protein, 117 aa, Artificial Sequence
  source: /note=“Description of Artificial Sequence: Synthetic polypeptide”
EVQLVESGGGLVQPGGSLRLSCAASGFTFSSYAMSWVRQAPGKGLEWVSAISVSGGSTYYADSVKGRFTISRDNSKNTLYLQMNSLRAEDTAVYYCAKANWGFFDYWGQGTLVTVSS

SEQ ID NO: 25 - protein, 106 aa, Artificial Sequence
  source: /note=“Description of Artificial Sequence: Synthetic polypeptide”
GQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 26 - protein, 328 aa, Artificial Sequence
  source: /note=“Description of Artificial Sequence: Synthetic polypeptide”
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVEPKSSDKTHTCPPCPAPPVAGPSVFLFPPKPKDTLMISRTPEVTCVVVDVSHEDPEVQFNWYVDGVEVHNAKTKPREEQAQSTFRVVSVLTVVHQDWLNGKEYKCAVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 27 - DNA, 326 bp, Artificial Sequence
  source: /note=“Description of Artificial Sequence: Synthetic polynucleotide”
cagagcgccctgacacagcctcgctcagtgtccgggtctcctggacagtcagtcaccatctcctgcactggaaccagcagtgatgttggtggttataactatgtctcctggtaccaacagcacccaggcaaagcccccaaactcatgatttacgatgtcagtaagcggccctcaggggtccctgatcgcttctctggctccaagtctggcaacacggcctccctgaccatctctgggctccaggctgaggatgaggctgattattactgctcctcatatgcagacagcgtggtattcggcggagggaccaaggtgaccgtcctagg

SEQ ID NO: 28 - DNA, 351 bp, Artificial Sequence
  source: /note=“Description of Artificial Sequence: Synthetic polynucleotide”
gaggtgcagctggtggagtctgggggaggcttggtacagcctggggggtccctgagactctcctgtgcagcctctggattcacctttagcagctatgccatgagctgggtccgccaggctccagggaaggggctggagtgggtctcagctattagtgttagtggtggtagcacatactacgcagactccgtgaagggccgattcaccatctccagagacaattccaagaacacgctgtatctgcaaatgaacagcctgagagccgaggacacggccgtatattactgtgcgaaagccaactgggggttctttgactactggggccagggaaccctggtcactgtctcttca

SEQ ID NO: 29 - DNA, 318 bp, Artificial Sequence
  source: /note=“Description of Artificial Sequence: Synthetic polynucleotide”
ggacagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctacctgagcctgacgcctgagcagtggaagtcccacaaaagctacagctgccaggtcacgcatgaagggagcaccgtggagaagacagtggcccctacagaatgttca

SEQ ID NO: 30 - DNA, 984 bp, Artificial Sequence
  source: /note=“Description of Artificial Sequence: Synthetic polynucleotide”
gctagcaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcccaaatcttctgacaaaactcacacatgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcaggcccagagcacgttccgtgtggtcagcgtcctcaccgttgtgcaccaggactggctgaacggcaaggagtacaagtgcgctgtctccaacaaaggcctcccagcccccatcgagaaaaccatctccaaaaccaaagggcagccccgagaaccacaggtgtacaccctgcccccatcacgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacacagaagagcctctccctgtccccgggt

SEQ ID NO: 31 - protein, 214 aa, Artificial Sequence
  source: /note=“Description of Artificial Sequence: Synthetic polypeptide”
SYELTQPRSVSGSPGQSVTISCTGTSSDVGGYNYVSWYQQHPGKAPKLMIFDVSKRPSGVPDRFSGSKSGNTASLTISGLQAEDEADYYCSSYADSVVFGGGTKVTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 32 - protein, 446 aa, Artificial Sequence
  source: /note=“Description of Artificial Sequence: Synthetic polypeptide”
EVQLVQSGGGLVQPGGSLRLSCAASGFTFSSYAMSWVRQAPGKGLEWVSAISVSGGSTYYADSVKGRFTISRDNSKNTLYLQMNSLRAEDTAVYYCAKANWGFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 33 - protein, 108 aa, Artificial Sequence
  source: /note=“Description of Artificial Sequence: Synthetic polypeptide”
SYELTQPRSVSGSPGQSVTISCTGTSSDVGGYNYVSWYQQHPGKAPKLMIFDVSKRPSGVPDRFSGSKSGNTASLTISGLQAEDEADYYCSSYADSVVFGGGTKVTVL

SEQ ID NO: 34 - protein, 117 aa, Artificial Sequence
  source: /note=“Description of Artificial Sequence: Synthetic polypeptide”
EVQLVQSGGGLVQPGGSLRLSCAASGFTFSSYAMSWVRQAPGKGLEWVSAISVSGGSTYYADSVKGRFTISRDNSKNTLYLQMNSLRAEDTAVYYCAKANWGFFDYWGQGTLVTVSS

SEQ ID NO: 35 - protein, 106 aa, Artificial Sequence; identical to SEQ ID NO: 25
  source: /note=“Description of Artificial Sequence: Synthetic polypeptide”

SEQ ID NO: 36 - protein, 329 aa, Artificial Sequence
  source: /note=“Description of Artificial Sequence: Synthetic polypeptide”
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 37 - DNA, 324 bp, Artificial Sequence
  source: /note=“Description of Artificial Sequence: Synthetic polynucleotide”
tcctatgagctgacacagcctcgctcagtgtccgggtctcctggacagtcagtcaccatctcctgcactggaaccagcagtgatgttggtggttataactatgtctcctggtaccaacagcacccaggcaaagcccccaaactcatgatttttgatgtcagtaagcggccctcaggggtccctgatcgcttctctggctccaagtctggcaacacggcctccctgaccatctctgggctccaggctgaggatgaggctgattattactgctcctcatatgcagacagcgtggtattcggcggagggaccaaggtgaccgtccta

SEQ ID NO: 38 - DNA, 351 bp, Artificial Sequence
  source: /note=“Description of Artificial Sequence: Synthetic polynucleotide”
gaggtgcagctggtgcagtctgggggaggcttggtacagcctggggggtccctgagactctcctgtgcagcctctggattcacctttagcagctatgccatgagctgggtccgccaggctccagggaaggggctggagtgggtctcagctattagtgttagtggtggtagcacatactacgcagactccgtgaagggccgattcaccatctccagagacaattccaagaacacgctgtatctgcaaatgaacagcctgagagccgaggacacggccgtatattactgtgcgaaagccaactgggggttctttgactactggggccagggaaccctggtcactgtctcttca

SEQ ID NO: 39 - DNA, 318 bp, Artificial Sequence; identical to SEQ ID NO: 29
  source: /note=“Description of Artificial Sequence: Synthetic polynucleotide”

SEQ ID NO: 40 - DNA, 987 bp, Artificial Sequence
  source: /note=“Description of Artificial Sequence: Synthetic polynucleotide”
gctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcacgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggt

SEQ ID NO: 41 - protein, 181 aa, Homo sapiens
SEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPAKVTPAPTRQRDFTAAFPRMLTTRGHGPAETQTLGSLPDINLTQISTLANELRDSRLANDLRDSGATIRIG

SEQ ID NO: 42 - protein, 174 aa, Macaca fascicularis
SEVEYIAEVGQNAYLPCSYTPAPPGNLVPVCWGKGACPVFDCSNVVLRTDNRDVNDRTSGRYWLKGDFHKGDVSLTIENVTLADSGVYCCRIQIPGIMNDEKHNVKLVVIKPAKVTPAPTLQRDLTSAFPRMLTTGEHGPAETQTPGSLPDVNLTQIFTLTNELRDSGATIRTA

SEQ ID NO: 43 - protein, 187 aa, Artificial Sequence
  source: /note=“Description of Artificial Sequence: Synthetic polypeptide”
SEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPAKVTPAPTLQRDFTAAFPRMLTTRGHGPAETQTLGSLPDINLTQISTLANELRDSRLANDLRDSGATIRVDHHHHHH

SEQ ID NO: 44 - protein, 426 aa, Artificial Sequence
  source: /note=“Description of Artificial Sequence: Synthetic polypeptide”
SEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPAKVTPAPTLQRDFTAAFPRMLTTRGHGPAETQTLGSLPDINLTQISTLANELRDSRLANDLRDSGATIRVDDIEGRMDEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKHHHHHH

SEQ ID NO: 45 - protein, 176 aa, Artificial Sequence
  source: /note=“Description of Artificial Sequence: Synthetic polypeptide”
EEYIVEVGQNAYLPCFYTLDTPGNLVPVCWGKGACPVFECGDVVLRTDERDVSYRTSSRYWLNGDFHKGNVTLAIGNVTLEDSGIYCCRVQIPGIMNDKKFNLKLVIKPAKVTPAPTLPRDSTPAFPRMLTTEDHGPAETQTLEILHDKNLTQLSTLANELQDAGTTIRIHHHHHH

SEQ ID NO: 46 - protein, 174 aa, Mus musculus
RSLENAYVFEVGKNAYLPCSYTLSTPGALVPMCWGKGFCPWSQCTNELLRTDERNVTYQKSSRYQLKGDLNKGDVSLIIKNVTLDDHGTYCCRIQFPGLMNDKKLELKLDIKAAKVTPAQTAHGDSTTASPRTLTTERNGSETQTLVTLHNNNGTKISTWADEIKDSGETIRTA

SEQ ID NO: 47 - protein, 229 aa, Artificial Sequence
  source: /note=“Description of Artificial Sequence: Synthetic polypeptide”
EVQLVESGGGLVQPGGSLRLSCAASGFTFSSYAMSWVRQAPGKGLEWVSAISVSGGSTYYADSVKGRFTISRDNSKNTLYLQMNSLRAEDTAVYYCAKANWGFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCAAAHHHHHH

SEQ ID NO: 48 - protein, 214 aa, Artificial Sequence; identical to SEQ ID NO: 31
  source: /note=“Description of Artificial Sequence: Synthetic polypeptide”

SEQ ID NO: 49 - protein, 110 aa, Artificial Sequence
  source: /note=“Description of Artificial Sequence: Synthetic polypeptide”
MSEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIK

SEQ ID NO: 50 - DNA, 333 bp, Artificial Sequence
  source: /note=“Description of Artificial Sequence: Synthetic polynucleotide”
atgagcgaggtggaatatcgggccgaagtgggccagaacgcctacctgccttgcttctacacaccagccgcccctggcaacctggtgcctgtgtgttggggaaagggcgcctgccctgtgttcgagtgcggcaacgtggtgctgagaaccgacgagcgggacgtgaactactggaccagccggtactggctgaacggcgacttcagaaagggcgacgtgtccctgaccatcgagaacgtgaccctggccgacagcggcatctactgctgcagaatccagatccccggcatcatgaacgacgagaagttcaacctgaagctcgtgatcaagtaa

SEQ ID NO: 51 - protein, 301 aa, Homo sapiens
MFSHLPFDCVLLLLLLLLTRSSEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPAKVTPAPTRQRDFTAAFPRMLTTRGHGPAETQTLGSLPDINLTQISTLANELRDSRLANDLRDSGATIRIGIYIGAGICAGLALALIFGALIFKWYSHSKEKIQNLSLISLANLPPSGLANAVAEGIRSEENIYTIEENVYEVEEPNEYYCYVSSRQQPSQPLGCRFAMP

SEQ ID NO: 52 - protein, 108 aa, Artificial Sequence
  source: /note=“Description of Artificial Sequence: Synthetic polypeptide”
QSALTQPRSVSGSPGQSVTISCTGTSSDVGGYNYVSWYQQHPGKAPKLMIFDVSKRPSGVPDRFSGSKSGNTASLTISGLQAEDEADYYCSSYADSVVFGGGTKVTVL

SEQ ID NO: 53 - protein, 117 aa, Artificial Sequence
  source: /note=“Description of Artificial Sequence: Synthetic polypeptide”
EVQLVQSGGGLVQPGGSLRLSCAASGFTFSSYALSWVRQAPGKGLEWVSAISVSGGSTYYADSVKGRFTISRDNSKNTLYLQMNSLRAEDTAVYYCAKANWGFFDYWGQGTLVTVSS

SEQ ID NO: 54 - protein, 108 aa, Artificial Sequence
  source: /note=“Description of Artificial Sequence: Synthetic polypeptide”
SYELTQPRSVSGSPGQSVTISCTGTSSDVGGYNYVSWYQQHPGKAPKLMIYDVSKRPSGVPDRFSGSKSGNTASLTISGLQAEDEADYYCSSYADSVVFGGGTKVTVL

SEQ ID NO: 55 - protein, 117 aa, Artificial Sequence
  source: /note=“Description of Artificial Sequence: Synthetic polypeptide”
EVQLVESGGGLVQPGGSLRLSCAASGFTFSSYALSWVRQAPGKGLEWVSAISVSGGSTYYADSVKGRFTISRDNSKNTLYLQMNSLRAEDTAVYYCAKANWGFFDYWGQGTLVTVSS

SEQ ID NO: 56-57 - skipped (2 entries)

SEQ ID NO: 58 - protein, 5 aa, Artificial Sequence
  source: /note=“Description of Artificial Sequence: Synthetic peptide”
  VARIANT (1)..(1): /replace=“Arg” or “Thr” or “Gln” or “Gly” or “Ala” or “Trp” or “Met” or “Ile” or “Ser”
  VARIANT (3)..(3): /replace=“Arg” or “Lys” or “Leu” or “Met” or “Ile”
  VARIANT (5)..(5): /replace=“Thr” or “Asn” or “Gln” or “Ala” or “Val” or “Tyr” or “Trp” or “Phe” or “Met”
  SITE (1)..(5): /note=“Variant residues given in the sequence have no preference with respect to those in the annotations for variant positions”
  source: /note=“See specification as filed for detailed description of substitutions and preferred embodiments”
KYVMH

SEQ ID NO: 59 - protein, 17 aa, Artificial Sequence
  source: /note=“Description of Artificial Sequence: Synthetic peptide”
  VARIANT (8)..(8): /replace=“Ile”
  VARIANT (14)..(14): /replace=“Thr”
  SITE (1)..(17): /note=“Variant residues given in the sequence have no preference with respect to those in the annotations for variant positions”
  source: /note=“See specification as filed for detailed description of substitutions and preferred embodiments”
SIYPSGGFTFYADSVKG

SEQ ID NO: 60 - protein, 11 aa, Artificial Sequence
  source: /note=“Description of Artificial Sequence: Synthetic peptide”
  VARIANT (10)..(10): /replace=“Asp”
  SITE (1)..(11): /note=“Variant residues given in the sequence have no preference with respect to those in the annotations for variant positions”
  source: /note=“See specification as filed for detailed description of substitutions and preferred embodiments”
IKLGTVTTVEY

SEQ ID NO: 61 - protein, 30 aa, Artificial Sequence
  source: /note=“Description of Artificial Sequence: Synthetic polypeptide”
EVQLLESGGGLVQPGGSLRLSCAASGFTFS

SEQ ID NO: 62 - protein, 14 aa, Artificial Sequence
  source: /note=“Description of Artificial Sequence: Synthetic peptide”
WVRQAPGKGLEWVS

SEQ ID NO: 63 - protein, 32 aa, Artificial Sequence
  source: /note=“Description of Artificial Sequence: Synthetic polypeptide”
RFTISRDNSKNTLYLQMNSLRAEDTAVYYCAR

SEQ ID NO: 64 - protein, 11 aa, Artificial Sequence
  source: /note=“Description of Artificial Sequence: Synthetic peptide”
WGQGTLVTVSS

SEQ ID NO: 65 - protein, 14 aa, Artificial Sequence
  source: /note=“Description of Artificial Sequence: Synthetic peptide”
  VARIANT (4)..(4): /replace=“Ser”
  VARIANT (5)..(5): /replace=“Arg” or “Ser”
  VARIANT (9)..(9): /replace=“Gly”
  SITE (1)..(14): /note=“Variant residues given in the sequence have no preference with respect to those in the annotations for variant positions”
  source: /note=“See specification as filed for detailed description of substitutions and preferred embodiments”
TGTNTDVGAYNYVS

SEQ ID NO: 66 - protein, 7 aa, Artificial Sequence
  source: /note=“Description of Artificial Sequence: Synthetic peptide”
  VARIANT (1)..(1): /replace=“Asp”
  VARIANT (3)..(3): /replace=“Asn” or “Ser”
  VARIANT (4)..(4): /replace=“His” or “Asn”
  SITE (1)..(7): /note=“Variant residues given in the sequence have no preference with respect to those in the annotations for variant positions”
  source: /note=“See specification as filed for detailed description of substitutions and preferred embodiments”
EVIDRPS

SEQ ID NO: 67 - protein, 10 aa, Artificial Sequence
  source: /note=“Description of Artificial Sequence: Synthetic peptide”
  VARIANT (3)..(3): /replace=“Tyr”
  VARIANT (5)..(5): /replace=“Ser”
  VARIANT (6)..(6): /replace=“Thr” or “Ser”
  VARIANT (7)..(7): /replace=“Ser”
  VARIANT (8)..(8): /replace=“Thr”
  SITE (1)..(10): /note=“Variant residues given in the sequence have no preference with respect to those in the annotations for variant positions”
  source: /note=“See specification as filed for detailed description of substitutions and preferred embodiments”
SSFTNRGIRV

SEQ ID NO: 68 - protein, 22 aa, Artificial Sequence
  source: /note=“Description of Artificial Sequence: Synthetic peptide”
QSALTQPASVSGSPGQSITISC

SEQ ID NO: 69 - protein, 15 aa, Artificial Sequence
  source: /note=“Description of Artificial Sequence: Synthetic peptide”
WYQQHPGKAPKLMIY

SEQ ID NO: 70 - protein, 32 aa, Artificial Sequence
  source: /note=“Description of Artificial Sequence: Synthetic polypeptide”
GVSNRFSGSKSGNTASLTISGLQAEDEADYYC

SEQ ID NO: 71 - protein, 10 aa, Artificial Sequence
  source: /note=“Description of Artificial Sequence: Synthetic peptide”
FGTGTKVTVL

SEQ ID NO: 72 - protein, 5 aa, Artificial Sequence
  source: /note=“Description of Artificial Sequence: Synthetic peptide”
  VARIANT (1)..(1): /replace=“Arg” or “Thr” or “Gln” or “Gly” or “Ala” or “Trp” or “Met” or “Ile” or “Ser”
  VARIANT (3)..(3): /replace=“Arg” or “Lys” or “Leu” or “Met” or “Ile”
  VARIANT (5)..(5): /replace=“Thr” or “Asn” or “Gln” or “Ala” or “Val” or “Tyr” or “Trp” or “Phe” or “Met”
  SITE (1)..(5): /note=“Variant residues given in the sequence have no preference with respect to those in the annotations for variant positions”
  source: /note=“See specification as filed for detailed description of substitutions and preferred embodiments”
KYVMH

SEQ ID NO: 73 - protein, 17 aa, Artificial Sequence
  source: /note=“Description of Artificial Sequence: Synthetic peptide”
  VARIANT (8)..(8): /replace=“Ile”
  VARIANT (14)..(14): /replace=“Thr”
  SITE (1)..(17): /note=“Variant residues given in the sequence have no preference with respect to those in the annotations for variant positions”
  source: /note=“See specification as filed for detailed description of substitutions and preferred embodiments”
SIYPSGGFTFYADSVKG

SEQ ID NO: 74 - protein, 11 aa, Artificial Sequence
  source: /note=“Description of Artificial Sequence: Synthetic peptide”
  VARIANT (10)..(10): /replace=“Asp”
  SITE (1)..(11): /note=“Variant residues given in the sequence have no preference with respect to those in the annotations for variant positions”
  source: /note=“See specification as filed for detailed description of substitutions and preferred embodiments”
IKLGTVTTVEY

SEQ ID NO: 75 - protein, 14 aa, Artificial Sequence
  source: /note=“Description of Artificial Sequence: Synthetic peptide”
  VARIANT (4)..(4): /replace=“Ser”
  VARIANT (5)..(5): /replace=“Arg” or “Ser”
  VARIANT (9)..(9): /replace=“Gly”
  SITE (1)..(14): /note=“Variant residues given in the sequence have no preference with respect to those in the annotations for variant positions”
  source: /note=“See specification as filed for detailed description of substitutions and preferred embodiments”
TGTNTDVGAYNYVS

SEQ ID NO: 76 - protein, 7 aa, Artificial Sequence
  source: /note=“Description of Artificial Sequence: Synthetic peptide”
  VARIANT (1)..(1): /replace=“Asp”
  VARIANT (3)..(3): /replace=“Asn” or “Ser”
  VARIANT (4)..(4): /replace=“His” or “Asn”
  SITE (1)..(7): /note=“Variant residues given in the sequence have no preference with respect to those in the annotations for variant positions”
  source: /note=“See specification as filed for detailed description of substitutions and preferred embodiments”
EVIDRPS

SEQ ID NO: 77 - protein, 10 aa, Artificial Sequence
  source: /note=“Description of Artificial Sequence: Synthetic peptide”
  VARIANT (3)..(3): /replace=“Tyr”
  VARIANT (5)..(5): /replace=“Ser”
  VARIANT (6)..(6): /replace=“Thr” or “Ser”
  VARIANT (7)..(7): /replace=“Ser”
  VARIANT (8)..(8): /replace=“Thr”
  SITE (1)..(10): /note=“Variant residues given in the sequence have no preference with respect to those in the annotations for variant positions”
  source: /note=“See specification as filed for detailed description of substitutions and preferred embodiments”
SSFTNRGIRV

SEQ ID NO: 78 - protein, 5 aa, Artificial Sequence
  source: /note=“Description of Artificial Sequence: Synthetic peptide”
SYIMM

SEQ ID NO: 79 - protein, 17 aa, Artificial Sequence
  source: /note=“Description of Artificial Sequence: Synthetic peptide”
SIYPSGGITFYADTVKG

SEQ ID NO: 80 - protein, 11 aa, Artificial Sequence
  source: /note=“Description of Artificial Sequence: Synthetic peptide”
IKLGTVTTVDY

SEQ ID NO: 81 - protein, 14 aa, Artificial Sequence
  source: /note=“Description of Artificial Sequence: Synthetic peptide”
TGTSSDVGGYNYVS

SEQ ID NO: 82 - protein, 7 aa, Artificial Sequence
  source: /note=“Description of Artificial Sequence: Synthetic peptide”
DVSNRPS

SEQ ID NO: 83 - protein, 10 aa, Artificial Sequence
  source: /note=“Description of Artificial Sequence: Synthetic peptide”
SSYTSSSTRV

SEQ ID NO: 84 - protein, 5 aa, Artificial Sequence
  source: /note=“Description of Artificial Sequence: Synthetic peptide”
MYMMM

SEQ ID NO: 85 - protein, 17 aa, Artificial Sequence
  source: /note=“Description of Artificial Sequence: Synthetic peptide”
SIYPSGGITFYADSVKG

SEQ ID NO: 86 - protein, 14 aa, Artificial Sequence
  source: /note=“Description of Artificial Sequence: Synthetic peptide”
TGTSSDVGAYNYVS

SEQ ID NO: 87 - protein, 119 aa, Artificial Sequence
  source: /note=“Description of Artificial Sequence: Synthetic polypeptide”
EVQLLESGGGLVQPGGSLRLSCAASGFTFSSYIMMVWRQAPGKGLEWVSSIYPSGGITFYADWKGRFTISRDNSKNTLYLQMNSLRAEDTAVYYCARIKLGTVTTVDYWGQGTLVTVSS

SEQ ID NO: 88 - protein, 110 aa, Artificial Sequence
  source: /note=“Description of Artificial Sequence: Synthetic polypeptide”
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVL

SEQ ID NO: 89 - protein, 120 aa, Artificial Sequence
  source: /note=“Description of Artificial Sequence: Synthetic polypeptide”
EVQLLESGGGLVQPGGSLRLSCAASGFTFSMYMMMWVRQAPGKGLEVWSSIYPSGGITFYADSVKGRFTISRDNSKNTLYLQMNSLRAEDTAIYYCARIKLGTVTTVDYWGQGTLVTVSS

SEQ ID NO: 90 - protein, 110 aa, Artificial Sequence
  source: /note=“Description of Artificial Sequence: Synthetic polypeptide”
QSALTQPASVSGSPGQSITISCTGTSSDVGAYNYVSWYQQHPGKAPKLMIYDVSNRPSGVSNRFSGSKSGNTASLTISGLQAEDEADYYCSSYTSSSTRVFGTGTKVTVL

SEQ ID NO: 91 - protein, 216 aa, Artificial Sequence
  source: /note=“Description of Artificial Sequence: Synthetic polypeptide”
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 92 - protein, 450 aa, Artificial Sequence
  source: /note=“Description of Artificial Sequence: Synthetic polypeptide”
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 93 - protein, 607 aa, Artificial Sequence
  source: /note=“Description of Artificial Sequence: Synthetic polypeptide”
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSGGGGSGIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 94 - DNA, 711 bp, Artificial Sequence
  source: /note=“Description of Artificial Sequence: Synthetic polynucleotide”
atgagggccctgctggctagactgctgctgtgcgtgctggtcgtgtccgacagcaagggccagtccgccctgacccagcctgcctccgtgtctggctcccctggccagtccatcaccatcagctgcaccggcacctccagcgacgtgggcggctacaactacgtgtcctggtatcagcagcaccccggcaaggcccccaagctgatgatctacgacgtgtccaaccggccctccggcgtgtccaacagattctccggctccaagtccggcaacaccgcctccctgaccatcagcggactgcaggcagaggacgaggccgactactactgctcctcctacacctcctccagcaccagagtgttcggcaccggcacaaaagtgaccgtgctgggccagcccaaggccaacccaaccgtgacactgttccccccatcctccgaggaactgcaggccaacaaggccaccctggtctgcctgatctcagatttctatccaggcgccgtgaccgtggcctggaaggctgatggctccccagtgaaggccggcgtggaaaccaccaagccctccaagcagtccaacaacaaatacgccgcctcctcctacctgtccctgacccccgagcagtggaagtcccaccggtcctacagctgccaggtcacacacgagggctccaccgtggaaaagaccgtcgcccccaccgagtgctcatga

SEQ ID NO: 95 - DNA, 1887 bp, Artificial Sequence
  source: /note=“Description of Artificial Sequence: Synthetic polynucleotide”
atggaaacagacaccctgctgctgtgggtgctgctgctgtgggtgcccggctccacaggcgaggtgcagctgctggaatccggcggaggactggtgcagcctggcggctccctgagactgtcttgcgccgcctccggcttcaccttctccagctacatcatgatgtgggtgcgacaggcccctggcaagggcctggaatgggtgtcctccatctacccctccggcggcatcaccttctacgccgacaccgtgaagggccggttcaccatctcccgggacaactccaagaacaccctgtacctgcagatgaactccctgcgggccgaggacaccgccgtgtactactgcgcccggatcaagctgggcaccgtgaccaccgtggactactggggccagggcaccctggtgacagtgtcctccgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtgctggcggcggaggaagcggaggaggtggcagcggtggcggtggctccggcggaggtggctccggaatccctccccacgtgcagaagtccgtgaacaacgacatgatcgtgaccgacaacaacggcgccgtgaagttccctcagctgtgcaagttctgcgacgtgaggttcagcacctgcgacaaccagaagtcctgcatgagcaactgcagcatcacaagcatctgcgagaagccccaggaggtgtgtgtggccgtgtggaggaagaacgacgaaaacatcaccctcgagaccgtgtgccatgaccccaagctgccctaccacgacttcatcctggaagacgccgcctcccccaagtgcatcatgaaggagaagaagaagcccggcgagaccttcttcatgtgcagctgcagcagcgacgagtgcaatgacaacatcatctttagcgaggagtacaacaccagcaaccccgactgataa

SEQ ID NO: 96 - protein, 136 aa, Homo sapiens
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 97 - protein, 21 aa, Artificial Sequence
  source: /note=“Description of Artificial Sequence: Synthetic peptide”
GGGGSGGGGSGGGGSGGGGSG

SEQ ID NO: 98 - protein, 4 aa, Artificial Sequence
  source: /note=“Description of Artificial Sequence: Synthetic peptide”
QFNS

SEQ ID NO: 99 - protein, 4 aa, Artificial Sequence
  source: /note=“Description of Artificial Sequence: Synthetic peptide”
QAQS

SEQ ID NO: 100 - protein, 6 aa, Artificial Sequence
  source: /note=“Description of Artificial Sequence: Synthetic peptide”
PKSCDK

SEQ ID NO: 101 - protein, 6 aa, Artificial Sequence
  source: /note=“Description of Artificial Sequence: Synthetic peptide”
PKSSDK

SEQ ID NO: 102 - protein, 4 aa, Artificial Sequence
  source: /note=“Description of Artificial Sequence: Synthetic peptide”
LSLS

SEQ ID NO: 103 - protein, 4 aa, Artificial Sequence
  source: /note=“Description of Artificial Sequence: Synthetic peptide”
ATAT

SEQ ID NO: 104 - protein, 117 aa, Homo sapiens
GAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 105 - protein, 115 aa, Homo sapiens
VKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 106 - protein, 122 aa, Homo sapiens [sequence omitted]

SEQ ID NO: 107 - protein, 110 aa, Homo sapiens
LCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 108 - protein, 122 aa, Artificial Sequence
  source: /note=“Description of Artificial Sequence: Synthetic polypeptide”
VTDNAGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 109 - protein, 118 aa, Artificial Sequence
  source: /note=“Description of Artificial Sequence: Synthetic polypeptide”
QVQLQESGPGLVKPSQTLSLTCTVSGGSISNDYWTWIRQHPGKGLEYIGYISYTGSTYYNPSLKSRVTISRDTSKNQFSLKLSSVTAADTAVYYCARSGGWLAPFDYWGRGTLVTVSS

SEQ ID NO: 110 - protein, 113 aa, Artificial Sequence
  source: /note=“Description of Artificial Sequence: Synthetic polypeptide”
DIVMTQSPDSLAVSLGERATINCKSSQSLFYHSNQKHSLAWYQQKPGQPPKLLIYGASTRESGVPDRFSGSGSGTDFTLTISSLQAEDVAVYYCQQYYGYPYTFGGGTKVEIK

SEQ ID NO: 111 - protein, 119 aa, Artificial Sequence
  source: /note=“Description of Artificial Sequence: Synthetic polypeptide”
QVQLVQSGAEVKKPGASVKVSCKASGYTFTSYWMHWVRQAPGQGLEWMGRIGPNSGFTSYNEKFKNRVTMTRDTSTSTVYMELSSLRSEDTAVYYCARGGSSYDYFDYWGQGTTVTVSS

SEQ ID NO: 112 - protein, 111 aa, Artificial Sequence
  source: /note=“Description of Artificial Sequence: Synthetic polypeptide”
DIVLTQSPASLAVSPGQRATITCRASESVSIHGTHLMHWYQQKPGQPPKLLIYAASNLESGVPARFSGSGSGTDFTLTINPVEAEDTANYYCQQSFEDPLTFGQGTKLEIK

SEQ ID NO: 113 - protein, 445 aa, Artificial Sequence
  source: /note=“Description of Artificial Sequence: Synthetic polypeptide”
QVQLQESGPGLVKPSQTLSLTCTVSGGSISNDYWTWIRQHPGKGLEYIGYISYTGSTYYNPSLKSRVTISRDTSKNQFSLKLSSVTAADTAVYYCARSGGWLAPFDYWGRGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 114 - protein, 220 aa, Artificial Sequence
  source: /note=“Description of Artificial Sequence: Synthetic polypeptide”
DIVMTQSPDSLAVSLGERATINCKSSQSLFYHSNQKHSLAWYQQKPGQPPKLLIYGASTRESGVPDRFSGSGSGTDFTLTISSLQAEDVAVYYCQQYYGYPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 115 - protein, 446 aa, Artificial Sequence
  source: /note=“Description of Artificial Sequence: Synthetic polypeptide”
QVQLVQSGAEVKKPGASVKVSCKASGYTFTSYWMHWVRQAPGQGLEWMGRIGPNSGFTSYNEKFKNRVTMTRDTSTSTVYMELSSLRSEDTAVYYCARGGSSYDYFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGA

SEQ ID NO: 116 - protein, 218 aa, Artificial Sequence
  source: /note=“Description of Artificial Sequence: Synthetic polypeptide”
DIVLTQSPASLAVSPGQRATITCRASESVSIHGTHLMHWYQQKPGQPPKLLIYAASNLESGVPARFSGSGSGTDFTLTINPVEAEDTANYYCQQSFEDPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 117 - protein, 26 aa, Artificial Sequence
  source: /note=“Description of Artificial Sequence: Synthetic peptide”
  SITE (1)..(25): /note=“This region may encompass 4-5 ′Gly Gly Gly Gly Ser′ repeating units”
GGGGSGGGGSGGGGSGGGGSGGGGSG

SEQ ID NO: 118 - protein, 108 aa, Artificial Sequence; identical to SEQ ID NO: 52
  source: /note=“Description of Artificial Sequence: Synthetic polypeptide”

SEQ ID NO: 119 - protein, 6 aa, Artificial Sequence
  source: /note=“Description of Artificial Sequence: Synthetic 6xHis tag”
HHHHHH

(19) United States
(12) Patent Application Publication
(10) Pub. No.: US 2022/0081480 A1
(43) Pub. Date: Mar. 17, 2022

(54) ANTI-TIM-3 ANTIBODIES

(71) Applicant: Merck Patent GmbH, Darmstadt (DE)

(72) Inventors: Dong Zhang, Westwood, MA (US); Xinyan Zhao, Bedford, MA (US); Qi An, Nashua, NH (US); David Nannemann, Woburn, MA (US); Rinat Zaynagetdinov, Bedford, MA (US); Vanita Sood, Somerville, MA (US); Christel Iffland, Arlington, MA (US)

(21) Appl. No.: 17/245,474

(22) Filed: Apr. 30, 2021

Related U.S. Application Data
(63) Continuation of application No. PCT/US2019/059555, filed on Nov. 1, 2019.
(60) Provisional application No. 62/754,383, filed on Nov. 1, 2018.

(51) Int. Cl. C07K 16/28; A61P 35/00

(52) U.S. Cl. CPC C07K 16/2803; A61K 2039/505; A61P 35/00; C07K 16/2827

(57) ABSTRACT

The invention is based, in part, upon the discovery of a family of antibodies that specifically bind human T Cell Immunoglobulin and Mucin Domain-3 (TIM-3). The antibodies contain TIM-3 binding sites based on the CDRs of the antibodies. The antibodies can be used as therapeutic agents as a monotherapy or in combination with another therapeutic agent. When used as therapeutic agents, the antibodies can be optimized, e.g., affinity-matured, to improve biochemical and/or biophysical properties and/or to reduce or eliminate immunogenicity, when administered to a human patient.

The antibodies inhibit TIM-3 from binding to TIM-3 ligands, e.g., galectin-9, phosphatidylserine (PtdSer) and carcinoembryonic antigen-related cell adhesion molecule 1 (CEACAM1). The disclosed antibodies can be used to inhibit the proliferation of tumor cells in vitro or in vivo. When administered to a human cancer patient or an animal model, the antibodies inhibit or reduce tumor growth in the human patient or animal model.

### CROSS-REFERENCE TO RELATED APPLICATIONS

This application claims the benefit of and priority to U.S. Provisional Patent Application No. 62/754,383, filed Nov. 1, 2018, the entire disclosures of which are incorporated by reference herein.

### FIELD OF THE INVENTION

The field of the invention is molecular biology, immunology and oncology. More particularly, the field is therapeutic antibodies.

### BACKGROUND

Following the approval of Ycrvoy® (ipilimumab, Bristol-Myers Squibb) for melanoma in 2011, immune checkpoint inhibitors have become a promising class of molecules for therapeutic development (for example, those targeting PD-1, PD-L1, and CTLA-4). Several large companies developing immune checkpoint inhibitor drugs include Bristol-Myers Squibb, Merck & Co., Roche, AstraZeneca and many others. The developmental strategies and investment in immunotherapy, together with compelling clinical efficacy have led to several new approvals of anti-PD(L)-1 drugs: Kcytruda® (pcmbrolizumab, Merck & Co.), Opdivo® (nivolumab, Bristol-Myers Squibb), Tecentriq® (atezolizumab, Roche), Bavencio® (avelumab, EMD Serono), and Imfinzi® (durvalumab, AstraZeneca).

PD-1/PD-L1 checkpoint inhibitors, with their compelling clinical efficacy and safety profiles, have built a solid foundation for combination immunotherapy approaches. These strategies include combining PD-1 pathway inhibitors with inhibitors of other immune checkpoint proteins expressed on T-cells. One such checkpoint protein is T Cell Immunoglobulin and Mucin Domain-3 (TIM-3), also known as Hepatitis A Virus Cellular Receptor 2 (HAVCR2).

Tim-3 was first identified as a molecule selectively expressed on IFN-g-producing CD4+ T helper 1(Th1) and CD8+ T cytotoxic 1 (Tc1) T cells (Monney et al. (2002) NATURE 415(6871):536-41). TIM-3 is also expressed on the surface of many immune cell types, including certain subsets of T cells such as FOXP3⁺CD4⁺ T regulatory cells (Tregs), natural killer (NK) cells, monocytes, and tumor-associated dendritic cells (TADCs) (Clayton et al. (2014) J. IMMUNOL. 192(2):782-791; Jones et al. (2008) J. EXP. MED. 205(12):2763-79; Hastings et al. (2009) EUR. J. IMMUNOL 39(9):2492-2501; Seki et al. (2008) CLIN IMMUNOL 127(1):78-88; Ju et al. (2010) J HEPATOL 52(3):322-329; Anderson et al. (2007) SCIENCE 318(5853):1141-1143; Baitsch et al. (2012) PLOS ONE 7(2):e30852; Ndhlovu et al. (2012) BLOOD 119(16):3734-3743). Putative ligands of TIM-3 have been reported, including phosphatidylserine (PtdSer; Nakayama et al., (2009) BLOOD 113(16):3821-30), galectin-9 (Gal-9) (Zhu et al. (2005) NAT IMMUNOL 6(12):1245-52), high-mobility group protein 1 (HMGB1) (Chiba et al. (2012) NAT IMMUNOL 13(9):832-42), and carcinoembryonic antigen cell adhesion molecule 1 (CEACAM1) (Huang et al. (2015) NATURE 517(7534):386-90).

Studies suggest that TIM-3 regulates various aspects of the immune response. The interaction of TIM-3 and its ligand galectin-9 (Gal-9) induces cell death. The in vivo blockade of this interaction exacerbated autoimmunity and abrogated tolerance in experimental models, suggesting that TIM-3/Gal-9 interaction negatively regulates immune responses (Zhu et al. (2005), supra; Kanzaki et al. (2012) ENDOCRINOLOGY 153(2):612-620). The inhibition of TIM-3 also enhanced the pathological severity in in vivo experimental autoimmune encephalomyelitis (Monney et al. (2002) NATURE 415:536-541; Das, et al. (2017) IMMUNOL REV 276(1):97-111). In studies using materials from human patients with multiple sclerosis (Koguchi et al. (2006) J EXP MED 203(6):1413-1418), Crohn's disease (CD) (Morimoto et al. (2011) SCAND J GASTROENTEROL 46(6):701-709) and rheumatoid arthritis (RA) (Liu et al. (2010) CLIN IMMUNOL 137(2):288-295; Li et al. (2014) PLoS ONE 9(2):e85784), the observation that Tim-3 expression level on T cells is inversely correlated with autoimmune disease progression suggests an immunosuppressive role of TIM-3 on T-cells. In addition to the effect on T-cells, TIM-3/Gal-9 interaction leads to antimicrobial activity by promoting macrophage clearance of intracellular pathogens (Sakuishi et al. (2011) TRENDS IMMUNOL 32(8):345-349), and TIM-3 may also promote clearance of apoptotic cells by binding phosphatidyl serine through its unique binding cleft (DeKruyff et al. (2010) J IMMUNOL 184(4):1918-1930).

Tim-3 is considered a potential candidate for cancer immunotherapy, in part, because it is upregulated in tumor-infiltrating lymphocytes including Foxp3+CD4+ Treg and exhausted CD8+ T cells, two key immune cell populations that constitute immunosuppression in tumor environment of many human cancers (McMahan et al. (2010) J. CLIN. INVEST. 120(12):4546-4557; Jin et al. (2010) PROC NATL ACAD SSCI USA 107(33):14733-8; Golden-Mason et al. (2009) J VIROL 83(18):9122-9130; Fourcade et al. (2010) J EXP MED 207(10):2175-86; Sakuishi et al. (2010) J EXP MED 207(10):2187-94; Zhou et al. (2011) BLOOD 117(17):4501-4510; Ngiow et al., (2011) CANCER RES. 71(10):3540-51, Yan, et al. (2013) PLoS ONE 8(3):e58006). The molecular mechanism of T cell dysregulation is hypothesized to begin with the interaction of Tim-3 on CD8+ T cells and its ligand galectin-9 on tumor cells, which results in the phosphorylation of the Tim-3 cytoplasmic tail at tyrosines 256 and 263, leading to the release of HLA-B-associated transcript 3 (Bat3) and catalytically active lymphocyte-specific protein tyrosine kinasc (Lck) from the Tim-3 cytoplasmic tail. The dissociation of Bat3 and Lck from Tim-3 leads to the accumulation of inactive phosphorylated Lck, which may account for the observed T cell dysfunction (Rangachari, et al. (2012) NAT MED 18(9):1394-400).

Further, intratumoral Tim-3+FoxP3+ Treg cells appear to express high amounts of Treg effector molecules (IL-10, perforin, and granzymes). Tim-3+ Tregs are thought to promote the development of a dysfunctional phenotype in CD8+ tumor infiltrating lymphocytes (TILs) in tumor environment (Sakuishi, el al. (2013) ONCOIMMUNOLOGY 2(4):e23849). Tim-3 has also been reported to have effects in the myeloid compartment. T-cell expression of Tim-3 has been shown to promote CD11b+Gr-1+ myeloid-derived suppressor cells (MDSC) in a galectin-9-dependent manner (Dardalhon, et al. (2010) J IMMUNOL 185(3):1383-92). Furthermore, as Tim-3 is specifically upregulated on tumor-associated dendritic cells (TADC), it is able to interfere with the sensing of DNA released by cells undergoing necrotic cell death. Tim-3 binds to high mobility group protein 1 (HMGB1), thereby prevents HMGB1 from binding to DNA released from dying cells and mediating delivery to innate cells via receptor for advanced glycation end (RAGE) products and/or Toll-like receptors (TLR) 2 and 4 pathways. Tim-3 binding to HMGB 1 dampens activation of the innate immune response in tumor tissue (Chiba, et al. (2012), supra). Taken together, these data suggest that Tim-3 can further suppress antitumor T-cell responses by T-cell extrinsic mechanisms involving myeloid cells and different Tim-3/ligand interactions.

The synergy of Tim-3/PD-1 co-blockade in inhibiting tumor growth in preclinical mouse tumor models suggests that the co-blockade modulates the functional phenotype of dysfunctional CD8+T cells and/or Tregs (Sakuishi et al. (2010), supra; Ngiow et al. (2011), supra). Indeed, besides in vivo co-blockade with PD(L)-1, co-blockade with many other check-point inhibitors enhances anti-tumor immunity and suppresses tumor growth in many preclinical tumor models (Dardalhon et al. (2010), supra; Nglow et al., CANCER RES 2011; Chiba et al. (2012), supra; Baghdadi et al., CANCER IMMUNOL IMMUNOTHER 2013; Kurtulus et al. (2015) J CLIN INVEST 125(11):4053-62; Huang et al. (2015), supra; Sakuishi et al. (2010), supra; Jing et al. (2015) J IMMUNOTHER CANCER 3:2; Zhou et al. (2011), supra; Komohara et al., CANCER IMMUNOLOGY RES., 2015).

Despite the success of checkpoint inhibitors such as Yervoy®, Keytruda® and Opdivo® and others, only a fraction of the patients experience durable clinical responses to these therapies. Some tumor types have shown little response to anti-CTLA-4 or anti-PD-1/PD-L1 monotherapies in clinical trials. These include prostate, colorectal, and pancreatic cancers. Accordingly, for these nonresponsive diseases and for the majority who are non-responders within responsive tumor types, there is a need for improved anti-tumor therapies.

### SUMMARY OF THE INVENTION

The invention is based, in part, upon the discovery of a family of antibodies that specifically bind human T Cell Immunoglobulin and Mucin Domain-3 (TIM-3). The antibodies contain TIM-3 binding sites based on the complementarity determining regions (CDRs) of the antibodies. The antibodies can be used as therapeutic agents alone or in combination with other therapeutic agents, such as other immune checkpoint inhibitors. When used as therapeutic agents, the antibodies can be optimized, e.g., affinity-matured, to improve biochemical properties (e.g., affinity and/or specificity), to improve biophysical properties (e.g., aggregation, stability, precipitation, and/or non-specific interactions), and/or to reduce or eliminate immunogenicity, when administered to a human patient.

The antibodies described herein inhibit TIM-3 from binding to TIM-3 ligands, e.g., galectin-9, phosphatidylserine (PtdSer), and carcinoembryonic antigen-related cell adhesion molecule 1 (CEACAM1). The disclosed antibodies can be used to inhibit the proliferation of tumor cells in vitro or in vivo. When administered to a human cancer patient or an animal model, the antibodies inhibit or reduce tumor growth in the human patient or animal model.

Accordingly, in one aspect, the disclosure relates to an isolated antibody that binds human TIM-3 comprising (i) an immunoglobulin heavy chain variable region comprising a CDR_{H1} comprising the amino acid sequence of SEQ ID NO: 1, a CDR_{H2} comprising the amino acid sequence of SEQ ID NO: 2, and a CDR_{H3} comprising the amino acid sequence of SEQ ID NO: 3; and (ii)an immunoglobulin light chain variable region comprising a CDR_{L1} comprising the amino acid sequence of SEQ ID NO: 4, a CDR_{L2} comprising the amino acid sequence of SEQ ID NO: 5, and a CDR_{L3} comprising the amino acid sequence of SEQ ID NO: 6.

In another aspect, the disclosure relates to an isolated antibody that binds human TIM-3, comprising an immunoglobulin heavy chain variable region selected from the group consisting of SEQ ID NO: 53, SEQ ID NO: 24, SEQ ID NO: 55, SEQ ID NO: 34, and an immunoglobulin light chain variable region selected from the group consisting of SEQ ID NO: 52, SEQ ID NO: 54, SEQ ID NO: 23 and SEQ ID NO: 33.

In another aspect, the disclosure relates to an isolated antibody that binds human TIM-3, comprising an immunoglobulin heavy chain variable region comprising the amino acid sequence of SEQ ID NO: 24, and an immunoglobulin light chain variable region comprising the amino acid sequence of SEQ ID NO: 23.

In another aspect, the disclosure relates to an isolated antibody that binds human TIM-3 comprising an immunoglobulin heavy chain and an immunoglobulin light chain selected from the group consisting of:

(a) an immunoglobulin heavy chain comprising the amino acid sequence of SEQ ID NO: 22, and an immunoglobulin light chain comprising the amino acid sequence of SEQ ID NO: 21; and

(b) an immunoglobulin heavy chain comprising the amino acid sequence of SEQ ID NO: 32, and an immunoglobulin light chain comprising the amino acid sequence of SEQ ID NO: 31.

In certain embodiments, the disclosure relates to an isolated nucleic acid comprising a nucleotide sequence encoding an immunoglobulin heavy chain variable region and/or immunoglobulin light chain variable region of the isolated antibody.

In certain embodiments, the disclosure relates to an expression vector comprising the nucleic acid encoding the immunoglobulin heavy chain variable region and/or immunoglobulin light chain variable region of the isolated antibody.

In certain embodiments, the disclosure relates to a cell comprising the expression vector comprising the nucleic acid encoding the immunoglobulin heavy chain variable region and/or immunoglobulin light chain variable region of the isolated antibody.

In certain embodiments, the disclosure relates to a method of producing a polypeptide comprising an immunoglobulin heavy chain variable region or an immunoglobulin light chain variable region, the method comprising (a) growing the host cell under conditions so that the host cell expresses the polypeptide comprising the immunoglobulin heavy chain variable region or the immunoglobulin light chain variable region; and (b) purifying the polypeptide comprising the immunoglobulin heavy chain variable region or the immunoglobulin light chain variable region.

In certain embodiments, the disclosure relates to a method of producing an antibody that binds human TIM-3 or an antigen binding fragment of the antibody, the method comprising (a) growing the host cell under conditions so that the host cell expresses a polypeptide comprising the immunoglobulin heavy chain variable region and the immunoglobulin light chain variable region, thereby producing the antibody or the antigen-binding fragment of the antibody; and (b) purifying the antibody or the antigen-binding fragment of the antibody.

In certain embodiments, the antibody has a KD of 9.2 nM or lower, as measured by surface plasmon resonance.

In certain embodiments, the disclosure relates to an isolated antibody that competes with an antibody as described herein for binding to the galectin-9 binding site on human TIM-3.

In certain embodiments, the disclosure relates to an isolated antibody that competes with an antibody as described herein for binding to the PtdSer binding site on human TIM-3.

In certain embodiments, the disclosure relates to an isolated antibody that competes with an antibody as described herein for binding to the carcinoembryonic antigen cell adhesion-related molecule 1 (CEACAM1) binding site on human TIM-3.

In certain embodiments, the disclosure relates to an isolated antibody that competes with an antibody as described herein for binding to the galectin-9 binding site, the PtdSer binding site, and the CEACAM1 binding site on human TIM-3.

In certain embodiments, the disclosure relates to an isolated antibody that binds to the same epitope on a human TIM-3 protein as an antibody as described herein, wherein the epitope includes P59, F61, E62, and D120 of the human TIM-3 protein.

In certain embodiments, the disclosure relates to a method of downregulating at least one exhaustion marker in a tumor microenvironment, the method comprising exposing the tumor microenvironment to an effective amount of an antibody as described herein to downregulate at least one exhaustion marker. In certain embodiments, the method further comprises exposing the tumor microenvironment to an effective amount of a second therapeutic agent, e.g., an anti-PD-L1 antibody. In certain embodiments, the exhaustion marker is CTLA-4, LAG-3, PD-1, or TIM-3.

In certain embodiments, the disclosure relates to a method of potentiating T cell activation, the method comprising exposing the T cell to an effective amount of an antibody as described herein, thereby to potentiate the activation of the T cell. In certain embodiments, the method further comprises exposing the T cell to an effective amount of a second therapeutic agent.

In certain embodiments, the disclosure relates to a method of inhibiting proliferation of a tumor cell comprising exposing the cell to an effective amount of an antibody as described herein to inhibit proliferation of the tumor cell. In certain embodiments, the method further comprises exposing the tumor cell to an effective amount of a second therapeutic agent.

In certain embodiments, the disclosure relates to a method of inhibiting tumor growth in a mammal, the method comprising exposing the mammal to an effective amount of an antibody as described herein to inhibit tumor growth in the mammal. In certain embodiments, the method further comprises exposing the mammal to an effective amount of a second therapeutic agent.

In certain embodiments, the disclosure relates to a method of treating cancer in a mammal, the method comprising administering an effective amount of the antibody as described herein to a mammal in need thereof. In certain embodiments, the method further comprises administering an effective amount of a second therapeutic agent to the mammal. In certain embodiments, the cancer is selected from the group consisting of diffuse large B-cell lymphoma, renal cell carcinoma (RCC), non-small cell lung carcinoma (NSCLC), squamous cell carcinoma of the head and neck (SCCHN), triple negative breast cancer (TNBC) or gastric/stomach adenocarcinoma (STAD). In certain embodiments, the mammal is a human.

In certain embodiments, the disclosure relates to an antibody as described herein for use in a method of downregulating at least one exhaustion marker in a tumor microenvironment, the method comprising exposing the tumor microenvironment to an effective amount of the antibody to downregulate at least one exhaustion marker. In certain embodiments, the method further comprises exposing the tumor microenvironment to an effective amount of a second therapeutic agent. In certain embodiments, the second therapeutic agent is an anti-PD-L1 antibody. In certain embodiments, the exhaustion marker is CTLA-4, LAG-3, PD-1, or TIM-3.

In certain embodiments, the disclosure relates to an antibody as described herein for use in a method of potentiating T cell activation, the method comprising exposing the T cell to an effective amount of the antibody, thereby to potentiate the activation of the T cell. In certain embodiments, the method further comprises exposing the T cell to an effective amount of a second therapeutic agent.

In certain embodiments, the disclosure relates to an antibody as described herein for use in a method of inhibiting proliferation of a tumor cell comprising exposing the cell to an effective amount of the antibody to inhibit proliferation of the tumor cell. In certain embodiments, the method further comprises exposing the tumor cell to an effective amount of a second therapeutic agent.

In certain embodiments, the disclosure relates to an antibody as described herein for use in a method of inhibiting tumor growth in a mammal, the method comprising exposing the mammal to an effective amount of the antibody to inhibit proliferation of the tumor. In certain embodiments, the method further comprises exposing the mammal to an effective amount of a second therapeutic agent.

In certain embodiments, the disclosure relates to an antibody as described herein for use in a method of treating cancer in a mammal, the method comprising administering an effective amount of the antibody to a mammal in need thereof In certain embodiments, the method further comprises administering an effective amount of a second therapeutic agent to the mammal. In certain embodiments, the cancer is selected from the group consisting of diffuse large B-cell lymphoma, renal cell carcinoma (RCC), non-small cell lung carcinoma (NSCLC), squamous cell carcinoma of the head and neck (SCCHN), triple negative breast cancer (TNBC) or gastric/stomach adenocarcinoma (STAD). In certain embodiments, the mammal is a human.

In certain embodiments, the disclosure relates to the use of an antibody as described herein for the manufacture of a medicament for downregulating at least one exhaustion marker in a tumor microenvironment, optionally in combination with a second therapeutic agent. In certain embodiments, the second therapeutic agent is an anti-PD-L1 antibody. In certain embodiments, the exhaustion marker is CTLA-4, LAG-3, PD-1, or TIM-3.

In certain embodiments, the disclosure relates to the use of an antibody as described herein for the manufacture of a medicament for potentiating T cell activation, optionally in combination with a second therapeutic agent.

In certain embodiments, the disclosure relates to the use of an antibody as described herein for the manufacture of a medicament for inhibiting proliferation of a tumor cell, optionally in combination with a second therapeutic agent.

In certain embodiments, the disclosure relates to the use of an antibody as described herein for the manufacture of a medicament for inhibiting tumor growth in a mammal, optionally in combination with a second therapeutic agent.

In certain embodiments, the disclosure relates to the use of an antibody as described herein for the manufacture of a medicament for treating cancer in a mammal, optionally in combination with a second therapeutic agent. In certain embodiments, the cancer is selected from the group consisting of diffuse large B-cell lymphoma, renal cell carcinoma (RCC), non-small cell lung carcinoma (NSCLC), squamous cell carcinoma of the head and neck (SCCHN), triple negative breast cancer (TNBC) or gastric/stomach adenocarcinoma (STAD).

In certain embodiments, the mammal is a human.

These and other aspects and advantages of the invention will become apparent upon consideration of the following figures, detailed description, and claims. As used herein, “including” means without limitation, and examples cited are non-limiting.

### BRIEF DESCRIPTION OF THE DRAWINGS

The foregoing and other objects, features and advantages of the invention will become apparent from the following description of preferred embodiments, as illustrated in the accompanying drawings. Like referenced elements identify common features in the corresponding drawings. The drawings are not necessarily to scale, with emphasis instead being placed on illustrating the principles of the present invention, in which:

FIG. 1 depicts the results of an ELISA assay showing anti-TIM-3 antibody 3903E11 (VL1.3,VH1.2) binding to human, cyno and marmoset TIM-3-His proteins.

FIG. 2A depicts the results of an ELISA competition assay, in which the anti-TIM-3 antibody 3903E11 (VL1.3,VH1.2) produced a dose-dependent blockade of binding between human galectin-9 and human TIM-3, with an IC50 of 2.4 nM. FIG. 2B depicts the results of an ELISA competition assay comparing competition with galectin-9 for a number of known anti-TIM-3 antibodies. FIG. 2C depicts the results of an ELISA competition assay, in which the anti-TIM-3 antibody 3903E11 (VL1.3,VH1.2) IgG2h (FN-AQ,322A)-delK (M6903), but not the isotype control antibody, inhibited huTTM-3-biotin binding to huGal-9 in a concentration-dependent manner, with an IC₅₀ of 7.46±0.052 nM. FIG. 2D depicts the results of an ELISA competition assay, in which the anti-TIM-3 antibody M6903, but not the isotype control antibody, inhibited rhTIM-3-Fc binding to His-tagged CEACAM1 in a dose-dependent manner, with an IC₅₀ of 0.353±0.383 nM (0.053±0.057 μg/mL).

FIGS. 3A-D shows the crystal structure of human TIM-3 in complex with M6903. FIG. 3A shows an overview of the Fab portion of M6903 (upper structure) bound to TIM-3 shown as a surface representation. Extensive contacts made on TIM-3 (bottom structure) arc shown as the lighter portion of TIM-3 . FIG. 3B shows the epitope hotspot residues of TIM-3 (e.g., P59 and F61 and E62). FIG. 3C shows the polar head group of ptdSer (light-colored sticks) and the coordinating calcium ion (sphere) have been modeled into the structure of M6903-bound TIM-3 by superposition with the structure of murinc TIM-3 (DeKruyff et al. (2010), supra). The binding site of ptdSer coincides with the placement of Y59 (group of spheres) of the heavy chain from M6903. Hydrogen bonds from D120 on TIM-3 to ptdSer or M6903, respectively, are shown as dotted lines. FIG. 3D shows the polar interactions of M6903 with the CEACAM-1 binding residues of TIM-3 are shown with dashed lines.

FIG. 4 depicts a model of the crystal structure of TIM-3 with an anti-TIM-3 antibody 3903E11 (VL1.3,VH1.2) epitope map showing the P59, F61, E62, 1114, N119, and K122 residues which reside on the face of one beta sheet of the immunoglobulin fold.

FIG. 5 provides a graph showing that target occupancy of anti-TIM-3 antibody M6903 on CD14⁺ monocytes increased with increased concentrations of anti-TIM-3. Serial dilutions of anti-TIM-3 antibody M6903 were incubated with fresh human whole blood for 1 hour. The unoccupied TIM-3 on CD14+ cells was measured by flow cytometry with anti-TIM-3 (2E2)-APC, which competes with the anti-TIM-3 antibody for TIM-3 binding. The average EC50 across all 10 donors was 111.1±85.6 ng/ml. The graph shows 4 representative donors (KP46233, KP46231, KP46315, and KP46318) out of the 10 total donors.

FIG. 6 provides a graph showing that M6903 efficiently blocked the interaction of rhTIM-3 and PtdSer on apoptotic Jurkat cells. Prior to flow cytometry analysis, apoptosis was induced in Jurkat cells via treatment with Staurosporine (2 μg/mL, 18 hrs), leading to surface expression of a TIM-3 ligand, PtdSer. Binding of rhTIM-3-Fc PtdSer on the surface of apoptotic Jurkat cells was evaluated via flow cytometry by measuring the MFI of rhTIM-3-Fc after pre-incubation with serial dilutions of M6903 or an anti-HEL IgG2h isotype control. While the isotype control had no effect, M6903 blocked the interaction of rhTIM-3 and PtdSer with an IC₅₀ of 4.438±3.115 nM (0.666±0.467 μg/ml). A nonlinear fit line was applied to the graph using a Sigmoid dose-response equation.

FIGS. 7A and 7B depict graphs showing M6903 increased CEF antigen specific T cell activation in a dose-dependent manner. The combination of M6903 and bintrafusp further enhanced this activation. PBMCs were treated with 40 μg/ml CEF viral peptide pool for (A) 6 days or (B) 4 days in the presence of M6903. In FIG. 7A, M6903 dose-dependently enhanced T cell activation compared to isotype control in a CEF assay as measured by IFN-γ production, with an EC50 of 1±1.3 μg/mL, calculated from multiple experiments. Non-linear regression analysis was performed and mean and SD are presented. In FIG. 7B, serial dilutions of M6903 were combined with either 10 μg/mL isotype control or bintrafusp alfa. The combination with bintrafusp alfa led to a further increase in IFN-γ production. Mean and SD are presented (p<0.05).

FIGS. 8A and 8B provide graphs showing M6903 dose-dependently enhancement of allo-antigen specific T cell activation. T cell activation was evaluated in an allogenic one-way MLR assay by measuring IFN-γ in the supernatant of co-cultured irradiated Daudi cells and human T cells after 2 days of treatment. In FIG. 8A, co-cultured cells were treated with serial dilutions of M6903 or isotype control. M6903 dose-dependently enhanced allo-antigen specific T cell activation, with an EC50 of 116±117 ng/mL. In FIG. 8B, co-cultured cells were treated with serial dilutions of M6903 combined with 10 μg/mL of isotype control, avelumab, or bintrafusp. The combination of M6903 with avelumab or bintrafusp further enhanced T cell activation. Nonlinear regression analysis was performed and mean±SD arc presented for both graphs.

FIGS. 9A and 9B provide a graph demonstrating that M6903 exhibits enhanced activity in combination with avelumab or bintrafusp in a superantigen SEB assay. Human PBMCs were treated with 100 ng/mL SEB along with 10 mg/mL M6903 (or isotype control) either alone or in combination with avelumab or bintrafusp alfa for 9 days. Cells were then washed once with medium and re-stimulated with SEB and the same antibodies for another 2 days. Supernatants were harvested and IFN-γ was measured by IFN-γ ELISA. M6903, avelumab, and bintrafusp alfa all increased IFN-γ production in SEB-stimulated T cells, and the effect was enhanced by combining M6903 with either of the other antibodies. FIG. 9A shows an experiment evaluating M6903 in combination with avelumab or bintrafusp alfa, and FIG. 9B shows an experiment evaluating M6903 in combination with avelumab only.

FIG. 10 depicts the results of a CEF antigen-specific T cell assay using M6903, anti-PdtSer, and anti-Gal9. PBMCs were treated with 40 μg/ml CEF viral peptide pool for 5 days in the presence of the antibody or antibodies indicated. The combination of anti-Gal-9 and anti-PtdSer had similar activity as M6903 alone, suggesting that blocking both Gal-9 and PtdSer may be required for anti-TIM-3 activity (compare data outlined by boxes).

FIGS. 11A-11B depict a quantitative analysis of TIM-3 expression measured via IHC in 12 tumor TMAs stained with anti-TIM-3 antibody. In FIG. 11A, the plot is ordered by median expression and in FIG. 11B, the plot is ordered by average expression following the removal of outliers.

FIG. 12 depicts mIF staining of 8 tumor tissues to identify immune cells expressing TIM-3 in the tumor microenvironment (TME). CD3 and CD68 were used as markers for lymphocytes and macrophages, respectively. The percentage of TIM-3⁺CD3⁺ lymphocytes and TIM-3⁺CD68⁺ macrophages was quantified across the tumor TMAs using mIF analysis.

FIG. 13 depicts TIM-3 expression in an NSCLC cohort using flow cytometry analysis. Within live CD3+ cells, expression of TIM-3 was observed to be highest on CD8+ T cells, followed by CD4+ T cells and Tregs. Each dot represents an individual sample. Lines represent the median value for each immune subset.

FIGS. 14A-C demonstrate that M6903 in combination with avelumab decreases expression of LAG-3, PD-1, and TIM-3 in TILs. huTIM-3 KI mice were inoculated with MC38 cells (3×10⁵) subcutaneously (s.c.) and treated with isotype control (20 mg/kg), avelumab (7 mg/kg), M6903 (10 mg/kg), or M6903+ avelumab for 6 days. Tumors were then analyzed via flow cytometry for the percentage of viable CD45+ cells out of total cells (FIG. 14A), the percentage of NK, NKT, CD3, CD4, CD8, or Treg cells in the CD45+ gate (FIG. 14B), and the percentage of CD4 and CD8 cells within the CD45+ gate co-expressing CTLA-4, LAG-3, PD-1, or TIM-3 (FIG. 14C). For each condition shown, the four bars presented are, from left to right: isotype control, avelumab, M6903, M6903+ avelumab. Values presented are mean+SEM. P-values were calculated with unpaired t-test analysis and denote significant differences between groups relative to isotype control; * P≤0.05, ** P≤0.01, *** P≤0.001, **** P≤0.0001.

FIGS. 15A-C demonstrate that M6903 and avelumab, as monotherapies or combination, decreased MC38 tumor volume in B-huTIM-3 KI mice. B-huTIM-3 KI mice were inoculated with MC38 (5×10⁵ cells) s.c. in the flank and then treated with isotype control (20 mg/kg), M6903 (10 mg/kg), avelumab (20 mg/kg) or M6903+ avelumab. FIG. 15A shows average tumor volumes with SEM and FIG. 15B shows individual tumor volumes. In FIG. 15C, survival was analyzed and median survival was calculated.

FIGS. 16A-B demonstrate that M6903 and bintrafusp alfa, as monotherapies or combination, decreased MC38 tumor volume in B-huTIM-3 KI mice. B-huTIM-3 KI mice were inoculated with MC38 (1×10⁶ cells) s.c. in the flank and then treated with isotype control (20 mg/kg), M6903 (10 mg/kg), bintrafusp alfa (24 mg/kg) or M6903+ bintrafusp alfa. FIG. 16A shows average tumor volumes with SEM and FIG. 16B shows individual tumor volumes.

### DETAILED DESCRIPTION

The anti-TIM-3 antibodies disclosed herein are based on the antigen binding sites of certain monoclonal antibodies that have been selected on the basis of binding and neutralizing the activity of human T Cell Immunoglobulin and Mucin Domain-3 (TIM-3). The antibodies contain immunoglobulin variable region CDR sequences that define a binding site for TIM-3.

In view of the neutralizing activity of these antibodies, they are useful for inhibiting the growth and/or proliferation of certain types of cancer cells. When used as a therapeutic agent, the antibodies can be optimized, e.g., affinity-matured, to improve biochemical properties and/or biophysical properties, and/or to reduce or eliminate immunogenicity when administered to a human patient. Various features and aspects of the invention are discussed in more detail below.

As used herein, unless otherwise indicated, the term “antibody” means an intact antibody (e.g., an intact monoclonal antibody) or antigen-binding fragment of an antibody, including an intact antibody or antigen-binding fragment of an antibody (e.g., a phage display antibody including a fully human antibody, a semisynthetic antibody or a fully synthetic antibody) that has been optimized, engineered or chemically conjugated. Examples of antibodies that have been optimized are affinity-matured antibodies. Examples of antibodies that have been engineered are Fc optimized antibodies, antibody fusion proteins and multispecific antibodies (e.g., bispecific antibodies). Examples of antigen-binding fragments include Fab, Fab′, F(ab′)₂, Fv, single chain antibodies (e.g., scFv), minibodies and diabodies. An antibody conjugated to a toxin moiety is an example of a chemically conjugated antibody. Antibody fusion proteins include, for example, an antibody genetically fused to a soluble ligand such as a cytokine, or to an extracellular domain of a cellular receptor protein.

I. Antibodies that Bind Human TIM-3

The antibodies disclosed herein comprise: (a) an immunoglobulin heavy chain variable region comprising a CDR_{H1}, a CDR_{H2}, and a CDR_{H3} and (b) an immunoglobulin light chain variable region comprising a CDR_{L1}, a CDR_{L2}, and a CDR_{L3}, wherein the heavy chain variable region and the light chain variable region together define a single binding site for binding TIM-3 protein.

In some embodiments, the antibody comprises: (a) an immunoglobulin heavy chain variable region comprising a CDR_{H1}, a CDR_{H2}, and a CDR_{H3} and (b) an immunoglobulin light chain variable region, wherein the heavy chain variable region and the light chain variable region together define a single binding site for binding TIM-3. A CDR_{H1} comprises the amino acid sequence of SEQ ID NO: 1; a CDR_{H2} comprises the amino acid sequence of SEQ ID NO: 2; and a CDR_{H3} comprises the amino acid sequence of SEQ ID NO: 3. The CDR_{H1}, CDR_{H2}, and CDR_{H3} sequences are interposed between immunoglobulin FR sequences (SEQ ID NO: 7, SEQ ID NO:8, SEQ ID NO: 9, and SEQ ID NO:10).

In some embodiments, the antibody comprises (a) an immunoglobulin light chain variable region comprising a CDR_{L1}, a CDR_{L2}, and a CDR_{L3}, and (b) an immunoglobulin heavy chain variable region, wherein the IgG light chain variable region and the IgG heavy chain variable region together define a single binding site for binding TIM-3. A CDR_{L1} comprises the amino acid sequence of SEQ ID NO: 4; a CDR_{L2} comprises the amino acid sequence of SEQ ID NO: 5; and a CDR_{L3} comprises the amino acid sequence of SEQ ID NO: 6. The CDR_{L1}, CDR_{L2}, and CDR_{L3} sequences are interposed between immunoglobulin FR sequences (SEQ ID NO: 11, SEQ ID NO: 12, SEQ ID NO: 13, and SEQ ID NO: 14).

In some embodiments, the antibody comprises: (a) an immunoglobulin heavy chain variable region comprising a CDR_{H1}, a CDR_{H2}, and a CDR_{H3} and (b) an immunoglobulin light chain variable region comprising a CDR_{L1}, a CDR_{L2}, and a CDR_{L3}, wherein the heavy chain variable region and the light chain variable region together define a single binding site for binding TIM-3. The CDR_{H1} is the amino acid sequence of SEQ ID NO: 1; the CDR_{H2} is the amino acid sequence of SEQ ID NO: 2; and the CDR_{H3} is the amino acid sequence of SEQ ID NO: 3. The CDR_{L1} is the amino acid sequence of SEQ ID NO: 4; the CDR_{L2} is the amino acid sequence of SEQ ID NO: 5; and the CDR_{L3} is the amino acid sequence of SEQ ID NO: 6.

In other embodiments, the antibodies disclosed herein comprise an immunoglobulin heavy chain variable region and an immunoglobulin light chain variable region. In some embodiments, the antibody comprises an immunoglobulin heavy chain variable region comprising an amino acid sequence selected from the group consisting of SEQ ID NO: 53, SEQ ID NO: 24, SEQ ID NO: 55, and SEQ ID NO: 34; and an immunoglobulin light chain variable region.

In other embodiments, the antibody comprises an immunoglobulin light chain variable region selected from the group consisting of SEQ ID NO: 52, SEQ ID NO: 54, SEQ ID NO: 23 and SEQ ID NO: 33; and an immunoglobulin heavy chain variable region.

In some embodiments, the antibody comprises an immunoglobulin heavy chain variable region comprising an amino acid sequence selected from the group consisting of SEQ ID NO: 53, SEQ ID NO: 24, SEQ ID NO: 55, and SEQ ID NO: 34; and all immunoglobulin light chain variable region selected from the group consisting of SEQ ID NO: 52, SEQ ID NO: 54, SEQ ID NO: 23 and SEQ ID NO: 33.

In some embodiments, the antibody comprises an immunoglobulin heavy chain variable region comprising the amino acid sequence of SEQ ID NO: 24, and an immunoglobulin light chain variable region comprising the amino acid sequence of SEQ ID NO: 23.

In certain embodiments, the antibodies disclosed herein comprise an immunoglobulin heavy chain and an immunoglobulin light chain. In some embodiments, the antibody comprises an immunoglobulin heavy chain selected from the group consisting of SEQ ID NO: 16, SEQ ID NO: 18, SEQ ID NO: 20, SEQ ID NO: 22, and SEQ ID NO: 32; and an immunoglobulin light chain.

In other embodiments, the antibody comprises an immunoglobulin light chain selected from the group consisting of SEQ ID NO: 15, SEQ ID NO: 17, SEQ ID NO: 19, SEQ ID NO: 21, and SEQ ID NO: 31; and an immunoglobulin heavy chain.

In some embodiments, the antibody comprises (i) an immunoglobulin heavy chain comprising an amino acid sequence selected from the group consisting of SEQ ID NO: 16, SEQ ID NO: 18, SEQ ID NO: 20, SEQ ID NO: 22, and SEQ ID NO: 32; and (ii) an immunoglobulin light chain selected from the group consisting of SEQ ID NO: 15, SEQ ID NO: 17, SEQ ID NO: 19, SEQ ID NO: 21, and SEQ ID NO: 31.

In some embodiments, the antibody comprises an immunoglobulin heavy chain comprising the amino acid sequence of SEQ ID NO: 22 and an immunoglobulin light chain comprising the amino acid sequence of SEQ ID NO: 21.

In certain embodiments, an isolated antibody that binds TIM-3 comprises an immunoglobulin heavy chain variable region comprising an amino acid sequence that is at least 70%, 75%, 80%, 85%, 90%, 95%, 98%, or 99% identical to the entire variable region or the framework region sequence of SEQ ID NO: 16, SEQ ID NO: 18, SEQ ID NO: 20, SEQ ID NO: 22, or SEQ ID NO: 32. In certain embodiments, an isolated antibody that binds TIM-3 comprises an immunoglobulin heavy chain variable region comprising a CDR_{H1} comprising the amino acid sequence of SEQ ID NO: 1; a CDR_{H2} comprising the amino acid sequence of SEQ ID NO: 2; and a CDR_{H3} comprising the amino acid sequence of SEQ ID NO: 3; and an amino acid sequence that is at least 70%, 75%, 80%, 85%, 90%, 95%, 98%, or 99% identical to the entire variable region or the framework region sequence of SEQ ID NO: 16, SEQ ID NO: 18, SEQ ID NO: 20, SEQ ID NO: 22, or SEQ ID NO: 32.

In certain embodiments, an isolated antibody that binds TIM-3 comprises an immunoglobulin light chain variable region comprising an amino acid sequence that is at least 70%, 75%, 80%, 85%, 90%, 95%, 98%, or 99% identical to the entire variable region or the framework region sequence of SEQ ID NO: 15, SEQ ID NO: 17, SEQ ID NO: 19, SEQ ID NO: 21, or SEQ ID NO: 31. In certain embodiments, an isolated antibody that binds TIM-3 comprises an immunoglobulin light chain variable region comprising a CDR_{L1} comprising the amino acid sequence of SEQ ID NO: 4; a CDR_{L2} comprising the amino acid sequence of SEQ ID NO: 5; and a CDR_{L3} comprising the amino acid sequence of SEQ ID NO: 6; and an amino acid sequence that is at least 70%, 75%, 80%, 85%, 90%, 95%, 98%, or 99% identical to the entire variable region or the framework region sequence of SEQ ID NO: 15, SEQ ID NO: 17, SEQ ID NO: 19, SEQ ID NO: 21, or SEQ ID NO: 31.

Sequence identity may be determined in various ways that are within the skill in the art, e.g., using publicly available computer software such as BLAST, BLAST-2, ALIGN or Megalign (DNASTAR) software. BLAST (Basic Local Alignment Search Tool) analysis using the algorithm employed by the programs blastp, blastn, blastx, tblastn and tblastx (Karlin et al., (1990) PROC. NATL. ACAD. SCI. USA 87:2264-2268; Altschul, (1993) J. MOL. EVOL. 36, 290-300; Altschul et al., (1997) NUCLEIC ACIDS RES. 25:3389-3402, incorporated by reference) are tailored for sequence similarity searching. For a discussion of basic issues in searching sequence databases see Altschul et al., (1994) NATURE GENETICS 6:119-129, which is fully incorporated by reference. Those skilled in the art can determine appropriate parameters for measuring alignment, including any algorithms needed to achieve maximal alignment over the full length of the sequences being compared. The search parameters for histogram, descriptions, alignments, expect (i.e., the statistical significance threshold for reporting matches against database sequences), cutoff, matrix and filter are at the default settings. The default scoring matrix used by blastp, blastx, tblastn, and tblastx is the BLO SUM62 matrix (Henikoff et al., (1992) PROC. NATL. ACAD. SCI. USA 89:10915-10919, fully incorporated by reference). Four blastn parameters may be adjusted as follows: Q=10 (gap creation penalty); R=10 (gap extension penalty); wink=1 (generates word hits at every wink.sup.th position along the query); and gapw=16 (sets the window width within which gapped alignments are generated). The equivalent Blastp parameter settings may be Q=9; R=2; wink=1; and gapw=32. Searches may also be conducted using the NCBI (National Center for Biotechnology Information) BLAST Advanced Option parameter (e.g.: -G, Cost to open gap [Integer]: default=5 for nucleotides/11 for proteins; -E, Cost to extend gap [Integer]: default=2 for nucleotides/1 for proteins; -q, Penalty for nucleotide mismatch [Integer]: default=−3; -r, reward for nucleotide match [Integer]: default=1; -e, expect value [Real]: default=10; -W, wordsize [Integer]: default=11 for nucleotides/28 for megablast/3 for proteins; -y, Dropoff (X) for blast extensions in bits: default=20 for blastn/7 for others; -X, X dropoff value for gapped alignment (in bits): default=15 for all programs, not applicable to blastn; and -Z, final X dropoff value for gapped alignment (in bits): 50 for blastn, 25 for others). ClustalW for pairwise protein alignments may also be used (default parameters may include, e.g., Blosum62 matrix and Gap Opening Penalty=10 and Gap Extension Penalty=0.1). A Bestfit comparison between sequences, available in the GCG package version 10.0, uses DNA parameters GAP=50 (gap creation penalty) and LEN=3 (gap extension penalty) and the equivalent settings in protein comparisons are GAP=8 and LEN=2.

In each of the foregoing embodiments, it is contemplated herein that immunoglobulin heavy chain variable region sequences and/or light chain variable region sequences that together bind TIM-3 may contain amino acid alterations (e.g., at least 1, 2, 3, 4, 5, or 10 amino acid substitutions, deletions, or additions) in the framework regions of the heavy and/or light chain variable regions. In certain embodiments, the amino acid alterations are conservative substitutions. As used herein, the term “conservative substitution” refers to a substitution with a structurally similar amino acid. For example, conservative substitutions may include those within the following groups: Ser and Cys; Leu, Ile, and Val; Glu and Asp; Lys and Arg; Phe, Tyr, and Trp; and Gln, Asn, Glu, Asp, and His. Conservative substitutions may also be defined by the BLAST (Basic Local Alignment Search Tool) algorithm, the BLOSUM substitution matrix (e.g., BLOSUM 62 matrix), or the PAM substitution:p matrix (e.g., the PAM 250 matrix).

In certain embodiments, the antibody binds TIM-3 with a KD of 20 nM, 15 nM, 10 nM, 9 nM, 8 nM, 7 nM, 6 nM, 5 nM, 4 nM, 3 nM, 2 nM, 1 nM or lower. Unless otherwise specified, KD values are determined by surface plasmon resonance, for example as described in Example 1.9.

In some embodiments, monoclonal antibodies bind to the same epitope on TIM-3 as any of the anti-TIM-3 antibodies disclosed herein (e.g., M6903). In some embodiments, monoclonal antibodies compete for binding to TIM-3 with any of the anti-TIM-3 antibodies disclosed herein. For example, monoclonal antibodies may compete for binding to the galectin-9 binding domain of TIM-3 with an anti-TIM-3 antibody described herein. In another example, monoclonal antibodies may compete for binding to the PtdSer binding domain of TIM-3 with an anti-TIM-3 antibody described herein. In another example, monoclonal antibodies may compete for binding to the CEACAM1 binding domain of TIM-3 with an anti-TIM-3 antibody described herein. In a further example, monoclonal antibodies may compete for binding to the galectin-9 binding domain and the PtdSer binding domain of TIM-3 with an anti-TIM-3 antibody described herein. In another example, monoclonal antibodies may compete for binding to the galectin-9 binding domain and the CEACAM1 binding domain of TIM-3 with an anti-TIM-3 antibody described herein. In another example, monoclonal antibodies may compete for binding to the PtdSer binding domain and the CEACAM1 binding domain of TIM-3 with an anti-TIM-3 antibody described herein. In another example, monoclonal antibodies may compete for binding to the galectin-9 binding domain, the PtdSer binding domain, and the CEACAM1 binding domain of TIM-3 with an anti-TIM-3 antibody described herein.

Competition assays for determining whether an antibody binds to the same epitope as an anti-TIM-3 antibody described herein, or competes for binding with galectin-9, PtdSer, and/or CEACAM1 with an anti-TIM-3 antibody described herein are known in the art. Exemplary competition assays include immunoassays (e.g., ELISA assays, RIA assays), BIAcore analysis, biolayer interferometry and flow cytometry.

Typically, a competition assay involves the use of an antigen (e.g., a TIM-3 protein or fragment thereof) bound to a solid surface or expressed on a cell surface, a test TIM-3-binding antibody and a reference antibody (e.g., antibody M6903). The reference antibody is labeled and the test antibody is unlabeled. Competitive inhibition is measured by determining the amount of labeled reference antibody bound to the solid surface or cells in the presence of the test antibody. Usually the test antibody is present in excess (e.g., 1×, 5×, 10×, 20× or 100×). Antibodies identified by competition assay (i.e., competing antibodies) include antibodies binding to the same epitope, or similar (e.g., overlapping) epitopes, as the reference antibody, and antibodies binding to an adjacent epitope sufficiently proximal to the epitope bound by the reference antibody for steric hindrance to occur.

In an exemplary competition assay, a reference TIM-3 antibody (e.g., antibody M6903) is biotinylated using commercially available reagents. The biotinylated reference antibody is mixed with serial dilutions of the test antibody or unlabeled reference antibody (self-competition control) resulting in a mixture of various molar ratios (e.g., 1×, 5×, 10×, 20× or 100×) of test antibody (or unlabeled reference antibody) to labeled reference antibody. The antibody mixture is added to a TIM-3 (e.g., TIM-3 extracellular domain) polypeptide coated-ELISA plate. The plate is then washed and HRP (horseradish peroxidase)-strepavidin is added to the plate as the detection reagent. The amount of labeled reference antibody bound to the target antigen is detected following addition of a chromogenic substrate (e.g., TMB (3,3′,5,5′-tetramethylbenzidine) or ABTS (2,2″-azino-di-(3-ethylbenzthiazoline-6-sulfonate)), which are well-known in the art. Optical density readings (OD units) are measured using a SpectraMax M2 spectrometer (Molecular Devices). OD units corresponding to zero percent inhibition are determined from wells without any competing antibody. OD units corresponding to 100% inhibition, i.e., the assay background are determined from wells without any labeled reference antibody or test antibody. Percent inhibition of labeled reference antibody to TIM-3 by the test antibody (or the unlabeled reference antibody) at each concentration is calculated as follows: % inhibition=(1-(OD units−100% inhibition)/(0% inhibition−100% inhibition))*100. Persons skilled in the art will appreciate that the competition assay can be performed using various detection systems well-known in the art.

A competition assay may be conducted in both directions to ensure that the presence of the label does not interfere or otherwise inhibit binding. For example, in the first direction the reference antibody is labeled and the test antibody is unlabeled, and in the second direction, the test antibody is labeled and the reference antibody is unlabeled.

A test antibody competes with the reference antibody for specific binding to the antigen if an excess of one antibody (e.g., 1×, 5×, 10×, 20× or 100×) inhibits binding of the other antibody, e.g., by at least 50%, 75%, 90%, 95% or 99% as measured in a competitive binding assay.

Two antibodies may be determined to bind to the same epitope if essentially all amino acid mutations in the antigen that reduce or eliminate binding of one antibody reduce or eliminate binding of the other. Two antibodies may be determined to bind to overlapping epitopes if only a subset of the amino acid mutations that reduce or eliminate binding of one antibody reduce or eliminate binding of the other.

### II. Production of Antibodies

Methods for producing antibodies, such as those disclosed herein, are known in the art. For example, DNA molecules encoding light chain variable regions and/or heavy chain variable regions can be chemically synthesized using the sequence information provided herein. Synthetic DNA molecules can be ligated to other appropriate nucleotide sequences, including, e.g., constant region coding sequences, and expression control sequences, to produce conventional gene expression constructs encoding the desired antibodies. Production of defined gene constructs is within routine skill in the art.

Nucleic acids encoding desired antibodies can be incorporated (ligated) into expression vectors, which can be introduced into host cells through conventional transfection or transformation techniques. Exemplary host cells are E. coli cells, Chinese hamster ovary (CHO) cells, human embryonic kidney 293 (HEK 293) cells, HeLa cells, baby hamster kidney (BHK) cells, monkey kidney cells (COS), human hepatocellular carcinoma cells (e.g., Hep G2), and myeloma cells that do not otherwise produce IgG protein. Transformed host cells can be grown under conditions that permit the host cells to express the genes that encode the immunoglobulin light and/or heavy chain variable regions.

Specific expression and purification conditions will vary depending upon the expression system employed. For example, if a gene is to be expressed in E. coli, it is first cloned into an expression vector by positioning the engineered gene downstream from a suitable bacterial promoter, e.g., Trp or Tac, and a prokaryotic signal sequence. The expressed secreted protein accumulates in refractile or inclusion bodies, and can be harvested after disruption of the cells by French press or sonication. The refractile bodies then are solubilized, and the proteins refolded and cleaved by methods known in the art.

If the engineered gene is to be expressed in eukaryotic host cells, e.g., CHO cells, it is first inserted into an expression vector containing a suitable eukaryotic promoter, a secretion signal, a poly A sequence, and a stop codon, and, optionally, may contain enhancers, and various introns. This expression vector optionally contains sequences encoding all or part of a constant region, enabling an entire, or a part of, a heavy or light chain to be expressed. The gene construct can be introduced into eukaryotic host cells using conventional techniques. The host cells express V_{L} or V_{H} fragments, V_{L}-V_{H} heterodimers, V_{H}-V_{L} or V_{L}-V_{H} single chain polypeptides, complete heavy or light immunoglobulin chains, or portions thereof, each of which may be attached to a moiety having another function (e.g., cytotoxicity). In some embodiments, a host cell is transfected with a single vector expressing a polypeptide expressing an entire, or part of, a heavy chain (e g., a heavy chain variable region) or a light chain (e g., a light chain variable region). In other embodiments, a host cell is transfected with a single vector encoding (a) a polypeptide comprising a heavy chain variable region and a polypeptide comprising a light chain variable region, or (b) an entire immunoglobulin heavy chain and an entire immunoglobulin light chain. In still other embodiments, a host cell is co-transfected with more than one expression vector (e.g., one expression vector expressing a polypeptide comprising an entire, or part of, a heavy chain or heavy chain variable region, and another expression vector expressing a polypeptide comprising an entire, or part of a light chain or light chain variable region).

A polypeptide comprising an immunoglobulin heavy chain variable region or light chain variable region can be produced by growing (culturing) a host cell transfected with an expression vector encoding such variable region, under conditions that permit expression of the polypeptide. Following expression, the polypeptide can be harvested and purified or isolated using techniques well known in the art, e.g., affinity tags such as glutathione-S-transferase (GST) and histidine tags.

A monoclonal antibody that binds human TIM-3, or an antigen-binding fragment of the antibody, can be produced by growing (culturing) a host cell transfected with: (a) an expression vector that encodes a complete or partial immunoglobulin heavy chain, and a separate expression vector that encodes a complete or partial immunoglobulin light chain; or (b) a single expression vector that encodes both chains (e.g., complete or partial heavy and light chains), under conditions that permit expression of both chains. The intact antibody (or antigen-binding fragment) can be harvested and purified or isolated using techniques well known in the art, e.g., Protein A, Protein G, affinity tags such as glutathione-S-transferase (GST) and histidine tags. It is within ordinary skill in the art to express the heavy chain and the light chain from a single expression vector or from two separate expression vectors.

### III. Antibody Modifications

Human monoclonal antibodies can be isolated or selected from phage display libraries including immune, naive and synthetic libraries. Antibody phage display libraries are known in the art, see, e.g., Hoet et al., NATURE BIOTECH. 23:344-348, 2005; Soderlind et al., NATURE BIOTECH. 18:852-856, 2000; Rothe et al., J. MOL. BIOL. 376:1182-1200, 2008; Knappik et al., J. MOL. BIOL. 296:57-86, 2000; and Krebs et al., J. IMMUNOL. METH. 254:67-84, 2001. When used as a therapeutic, human antibodies isolated by phage display may be optimized (e.g., affinity-matured) to improve biochemical characteristics including affinity and/or specificity, improve biophysical properties including aggregation, stability, precipitation and/or non-specific interactions, and/or to reduce immunogenicity. Affinity-maturation procedures are within ordinary skill in the art. For example, diversity can be introduced into an immunoglobulin heavy chain and/or an immunoglobulin light chain by DNA shuffling, chain shuffling, CDR shuffling, random mutagenesis and/or site-specific mutagenesis.

In some embodiments, isolated human antibodies contain one or more somatic mutations in a framework region. In these cases, framework regions can be modified to a human germline sequence to optimize the antibody (i.e., a process referred to as germlining).

Generally, an optimized antibody has at least the same, or substantially the same, affinity for the antigen as the non-optimized (or parental) antibody from which it was derived. Preferably, an optimized antibody has a higher affinity for the antigen when compared to the parental antibody.

Antibody Fragments

The proteins and polypeptides of the invention can also include antigen-binding fragments of antibodies. Exemplary antibody fragments include scFv, Fv, Fab, F(ab′)₂, and single domain VHH fragments such as those of camelid origin.

Single-chain antibody fragments, also known as single-chain antibodies (scFvs), are recombinant polypeptides which typically bind antigens or receptors; these fragments contain at least one fragment of an antibody variable heavy-chain amino acid sequence (V_{H}) tethered to at least one fragment of an antibody variable light-chain sequence (V_{L}) with or without one or more interconnecting linkers. Such a linker may be a short, flexible peptide selected to assure that the proper three-dimensional folding of the V_{L} and V_{H} domains occurs once they are linked so as to maintain the target molecule binding-specificity of the whole antibody from which the single-chain antibody fragment is derived. Generally, the carboxyl terminus of the V_{L} or V_{H} sequence is covalently linked by such a peptide linker to the amino acid terminus of a complementary V_{L} and V_{H} sequence. Single-chain antibody fragments can be generated by molecular cloning, antibody phage display library or similar techniques. These proteins can be produced either in cukaryotic cells or prokaryotic cells, including bacteria.

Single-chain antibody fragments contain amino acid sequences having at least one of the variable regions or complementarity determining regions (CDRs) of the whole antibodies described in this specification, but are lacking some or all of the constant domains of those antibodies. These constant domains are not necessary for antigen binding, but constitute a major portion of the structure of whole antibodies. Single-chain antibody fragments may therefore overcome some of the problems associated with the use of antibodies containing part or all of a constant domain. For example, single-chain antibody fragments tend to be free of undesired interactions between biological molecules and the heavy-chain constant region, or other unwanted biological activity. Additionally, single-chain antibody fragments are considerably smaller than whole antibodies and may therefore have greater capillary permeability than whole antibodies, allowing single-chain antibody fragments to localize and bind to target antigen-binding sites more efficiently. Also, antibody fragments can be produced on a relatively large scale in prokaryotic cells, thus facilitating their production. Furthermore, the relatively small size of single-chain antibody fragments makes them less likely than whole antibodies to provoke an immune response in a recipient.

Fragments of antibodies that have the same or comparable binding characteristics to those of the whole antibody may also be present. Such fragments may contain one or both Fab fragments or the F(ab′)₂ fragment. The antibody fragments may contain all six CDRs of the whole antibody, although fragments containing fewer than all of such regions, such as three, four or five CDRs, are also functional.

Constant Regions

Unless otherwise specified, constant region antibody amino acid residues are numbered according to the Kabat EU index in Kabat, E. A. et al., (Sequences of proteins of immunological interest. 5th Edition—US Department of Health and Human Services, NIH publication n° 91-3242, pp 662,680,689 (1991)). The antibodies and fragments thereof (e.g., parental and optimized variants) as described herein can be engineered to contain certain constant (i.e., Fc) regions with or lacking a specified effector function (e.g., antibody-dependent cellular cytotoxicity (ADCC)). Human constant regions are known in the art.

The proteins and peptides (e.g., antibodies) of the invention can include a constant region of an immunoglobulin or a fragment, analog, variant, mutant, or derivative of the constant region. In preferred embodiments, the constant region is derived from a human immunoglobulin heavy chain, for example, IgG1, IgG2, IgG3, IgG4, or other classes. In one embodiment, the constant region includes a CH2 domain. In another embodiment, the constant region includes CH2 and CH3 domains or includes hinge-CH2-CH3. Alternatively, the constant region can include all or a portion of the hinge region, the CH2 domain and/or the CH3 domain.

In one embodiment, the constant region contains a mutation that reduces affinity for an Fc receptor or reduces Fc effector function. For example, the constant region can contain a mutation that eliminates the glycosylation site within the constant region of an IgG heavy chain. In some embodiments, the constant region contains mutations, deletions, or insertions at an amino acid position corresponding to Leu234, Leu235, Gly236, Gly237, Asn297, or Pro331 of IgG1 (amino acids are numbered according to Kabat EU index). In a particular embodiment, the constant region contains a mutation at an amino acid position corresponding to Asn297 of IgG1. In alternative embodiments, the constant region contains mutations, deletions, or insertions at an amino acid position corresponding to Leu281, Leu282, Gly283, Gly284, Asn344, or Pro378 of IgG 1.

In some embodiments, the constant region contains a CH2 domain derived from a human IgG2 or IgG4 heavy chain Preferably, the CH2 domain contains a mutation that eliminates the glycosylation site within the CH2 domain. In one embodiment, the mutation alters the asparagine within the Gln-Phe-Asn-Ser (SEQ ID NO: 98) amino acid sequence within the CH2 domain of the IgG2 or IgG4 heavy chain. Preferably, the mutation changes the asparagine to a glutamine. Alternatively, the mutation alters both the phenylalanine and the asparagine within the Gln-Phe-Asn-Ser (SEQ ID NO: 98) amino acid sequence. In one embodiment, the Gln-Phe-Asn-Ser (SEQ ID NO: 98) amino acid sequence is replaced with a Gln-Ala-Gln-Ser (SEQ ID NO: 99) amino acid sequence. The asparagine within the Gln-Phe-Asn-Ser (SEQ ID NO: 98) amino acid sequence corresponds to Asn297 of IgG1 (Kabat EU index).

In another embodiment, the constant region includes a CH2 domain and at least a portion of a hinge region. The hinge region can be derived from an immunoglobulin heavy chain, e.g., IgG1, IgG2, IgG3, IgG4, or other classes. Preferably, the hinge region is derived from human IgG1, IgG2, IgG3, IgG4, or other suitable classes. More preferably the hinge region is derived from a human IgG1 heavy chain In one embodiment the cysteine in the Pro-Lys-Ser-Cys-Asp-Lys (SEQ ID NO: 100) amino acid sequence of the IgG1 hinge region is altered. In a preferred embodiment the Pro-Lys-Scr-Cys-Asp-Lys (SEQ ID NO: 100) amino acid sequence is replaced with a Pro-Lys-Ser-Ser-Asp-Lys (SEQ ID NO: 101) amino acid sequence. In one embodiment, the constant region includes a CH2 domain derived from a first antibody isotype and a hinge region derived from a second antibody isotype. In a specific embodiment, the CH2 domain is derived from a human IgG2 or IgG4 heavy chain, while the hinge region is derived from an altered human IgG1 heavy chain.

The alteration of amino acids near the junction of the Fc portion and the non-Fc portion of an antibody or Fc fusion protein can dramatically increase the serum half-life of the Fc fusion protein (PCT publication WO 01/58957, the disclosure of which is hereby incorporated by reference). Accordingly, the junction region of a protein or polypeptide of the present invention can contain alterations that, relative to the naturally-occurring sequences of an immunoglobulin heavy chain, preferably lie within about 10 amino acids of the junction point. These amino acid changes can cause an increase in hydrophobicity. In one embodiment, the constant region is derived from an IgG sequence in which the C-terminal lysine residue is replaced. Preferably, the C-terminal lysine of an IgG sequence is replaced with a non-lysine amino acid, such as alanine or leucine, to further increase serum half-life. In another embodiment, the constant region is derived from an IgG sequence in which the Leu-Ser-Leu-Ser (SEQ ID NO: 102) amino acid sequence near the C-terminus of the constant region is altered to eliminate potential junctional T-cell epitopes. For example, in one embodiment, the Leu-Ser-Leu-Ser (SEQ ID NO: 102) amino acid sequence is replaced with an Ala-Thr-Ala-Thr (SEQ ID NO: 103) amino acid sequence. In other embodiments, the amino acids within the Leu-Ser-Leu-Ser (SEQ ID NO: 102) segment are replaced with other amino acids such as glycine or proline. Detailed methods of generating amino acid substitutions of the Leu-Ser-Leu-Ser (SEQ ID NO: 102) segment near the C-terminus of an IgG1, IgG2, IgG3, IgG4, or other immunoglobulin class molecule have been described in U.S. Patent Publication No. 2003/0166877, the disclosure of which is hereby incorporated by reference.

Suitable hinge regions for the present invention can be derived from IgG1, IgG2, IgG3, IgG4, and other immunoglobulin classes. The IgG1 hinge region has three cysteines, two of which are involved in disulfide bonds between the two heavy chains of the immunoglobulin. These same cysteines permit efficient and consistent disulfide bonding formation between Fc portions. Therefore, a preferred hinge region of the present invention is derived from IgG1, more preferably from human IgG1. In some embodiments, the first cysteine within the human IgG1 hinge region is mutated to another amino acid, preferably serine. The IgG2 isotype hinge region has four disulfide bonds that tend to promote oligomerization and possibly incorrect disulfide bonding during secretion in recombinant systems. A suitable hinge region can be derived from an IgG2 hinge; the first two cysteines are each preferably mutated to another amino acid. The hinge region of IgG4 is known to form interchain disulfide bonds inefficiently. However, a suitable hinge region for the present invention can be derived from the IgG4 hinge region, preferably containing a mutation that enhances correct formation of disulfide bonds between heavy chain-derived moieties (Angal S, et al. (1993) Mol. Immunol., 30:105-8).

In accordance with the present invention, the constant region can contain CH2 and/or CH3 domains and a hinge region that are derived from different antibody isotypes, i.e., a hybrid constant region. For example, in one embodiment, the constant region contains CH2 and/or CH3 domains derived from IgG2 or IgG4 and a mutant hinge region derived from IgG1. Alternatively, a mutant hinge region from another IgG subclass is used in a hybrid constant region. For example, a mutant form of the IgG4 hinge that allows efficient disulfide bonding between the two heavy chains can be used. A mutant hinge can also be derived from an IgG2 hinge in which the first two cysteines are each mutated to another amino acid. Assembly of such hybrid constant regions has been described in U.S. Patent Publication No. 2003/0044423, the disclosure of which is hereby incorporated by reference.

In accordance with the present invention, the constant region can contain one or more mutations described herein. The combinations of mutations in the Fc portion can have additive or synergistic effects on the prolonged serum half-life and increased in vivo potency of the molecule. Thus, in one exemplary embodiment, the constant region can contain (i) a region derived from an IgG sequence in which the Leu-Ser-Leu-Ser (SEQ ID NO: 102) amino acid sequence is replaced with an Ala-Thr-Ala-Thr (SEQ ID NO: 103) amino acid sequence; (ii) a C-terminal alanine residue instead of lysine; (iii) a CH2 domain and a hinge region that are derived from different antibody isotypes, for example, an IgG2 CH2 domain and an altered IgG1 hinge region; and (iv) a mutation that eliminates the glycosylation site within the IgG2-derived CH2 domain, for example, a Gln-Ala-Gln-Ser (SEQ ID NO: 99) amino acid sequence instead of the Gln-Phe-Asn-Ser (SEQ ID NO: 98) amino acid sequence within the IgG2-derived CH2 domain.

If the antibody is for use as a therapeutic, it can be conjugated to an effector agent such as a small molecule toxin or a radionuclide using standard in vitro conjugation chemistries. If the effector agent is a polypeptide, the antibody can be chemically conjugated to the effector agent or joined to the effector agent as a fusion protein. Construction of fusion proteins is within ordinary skill in the art.

### IV. Use of Antibodies

The antibodies described herein can be used in a method of downregulating at least one exhaustion marker in a tumor microenvironment, the method comprising exposing the tumor microenvironment to an effective amount of an anti-TIM-3 antibody to downregulate at least one exhaustion marker, such as CTLA-4, LAG-3, PD-1, or TIM-3. Methods for measuring downregulation of exhaustion markers are described in Example 6.2. In certain embodiments, the method can further include exposing the tumor microenvironment to an effective amount of a second therapeutic agent, such as an immune checkpoint inhibitor. Examples of immune checkpoint inhibitors include inhibitors targeting PD-1, PD-L1, or CTLA-4, such as an anti-PD-L1 antibody (e.g., avelumab).

The antibodies described herein also can be used in a method of potentiating T cell activation. The method can include exposing the T cell to an effective amount of an anti-TIM-3 antibody, thereby to potentiate the activation of the T cell. In certain embodiments, the method further includes exposing the T cell to an effective amount of a second therapeutic agent, such as an immune checkpoint inhibitor. Methods for measuring T cell activation are described in Example 5.3, and can include measuring IFN-γ production from human PBMCs that were activated by exposure to CEF antigens. In certain embodiments, the method can further include exposing the tumor microenvironment to an effective amount of a second therapeutic agent, such as an anti-PD-L1 antibody (e.g., avelumab).

The antibodies disclosed herein can be used to treat various forms of cancer. In certain embodiments, the cancer or tumor may be selected from the group consisting of colorectal, breast, ovarian, pancreatic, gastric, prostate, renal, cervical, myeloma, lymphoma, leukemia, thyroid, endometrial, uterine, bladder, neuroendocrine, head and neck, liver, nasopharyngeal, testicular, small cell lung cancer, non-small cell lung cancer, melanoma, basal cell skin cancer, squamous cell skin cancer, dermatofibrosarcoma protuberans, Merkel cell carcinoma, glioblastoma, glioma, sarcoma, mesothelioma, and myelodysplastic syndromes. In certain embodiments, the cancer is diffuse large B-cell lymphoma, renal cell carcinoma (RCC), non-small cell lung carcinoma (NSCLC), squamous cell carcinoma of the head and neck (SCCHN), triple negative breast cancer (TNBC) or gastric/stomach adenocarcinoma (STAD). In certain embodiments, the cancer is metastatic or a locally advanced solid tumor. In certain embodiments, no standard therapy exists to treat the cancer and/or the cancer is relapsed and/or refractory from at least one prior treatment. The cancer cells are exposed to a therapeutically effective amount of the antibody so as to inhibit proliferation of the cancer cell. In some embodiments, the antibodies inhibit cancer cell proliferation by at least 40%, 50%, 60%, 70%, 80%, 90%, 95%, 98%, 99%, or 100%.

In some embodiments, the anti-TIM-3 antibody is used in therapy. For example, the antibody can be used to inhibit tumor growth in a mammal (e.g., a human patient). In some embodiments, use of the antibody to inhibit tumor growth in a mammal comprises administering to the mammal a therapeutically effective amount of the antibody. In other embodiments, the anti-TIM-3 antibody can be used for inhibiting proliferation of a tumor cell.

In some embodiments, the anti-TIM-3 antibody is administered in combination with another therapeutic agent, such as radiation (e.g., stereotactic radiation) or an immune checkpoint inhibitor (e.g., targeting PD-1, PD-L1, or CTLA-4). In some embodiments, the anti-TIM-3 antibody is administered in combination with one or more of the following therapeutic agents: anti-PD1/anti-PD-L1 antibodies including Keytruda® (pembrolizumab, Merck & Co.), Opdivo® (nivolumab, Bristol-Myers Squibb), Tecentriq® (atezolizumab, Roche), Bavencio® (avelumab, EMD Serono,), Imfinzi® (durvalumab, AstraZeneca), TGF-β pathway targeting agents including galunisertib (LY2157299 monohydrate, a small molecule kinase inhibitor of TGF-βRI), LY3200882 (a small molecule kinase inhibitor TGF-βRI disclosed by Pei et al. (2017) CANCER RES 77(13 Suppl):Abstract 955), Metelimumab (an antibody targeting TGF-β1, see Colak et al. (2017) TRENDS CANCER 3(1):56-71), Fresolimumab (GC-1008; an antibody targeting TGF-β1 and TGF-β2), XOMA 089 (an antibody targeting TGF-β1 and TGF-β2; see Mirza et al. (2014) INVESTIGATIVE OPHTHALMOLOGY & VISUAL SCIENCE 55:1121), AVTD200 (a TGF-β1 and TGF-β3 trap, see Thwaites et al. (2017) BLOOD 130:2532), Trabedersen/AP12009 (a TGF-β2 antisense oligonucleotide, see Jaschinski et al. (2011) CURR PHARM BIOTECHNOL. 12(12):2203-13), Belagen-pumatucel-L (a tumor cell vaccine targeting TGF-β2, see, e.g., Giaccone et al. (2015) EUR J CANCER 51(16):2321-9); TGB-β pathway targeting agents described in Colak et al. (2017), supra, including Ki26894, SD208, SM16, IMC-TR1, PF-03446962, TEW-7197, and GW788388; any of the immunomodulatory antibodies and fusion proteins described in International Patent Publication No. WO 2011/109789, including those with an immunomodulatory moiety binding to TGF-β, TGF-βR, PD-L1, PD-L2, PD-1, Receptor activator of nuclear factor-κB (RANK) ligand (RANKL), and Receptor activator of nuclear factor-κB (RANK), such as the anti-HER2/neu antibody and TGFβRII ECD fusion protein comprising SEQ ID Nos: 1 and 70 (SEQ ID Nos referenced in the following list are the sequence identifiers as disclosed in International Patent Publication No. WO 2011/109789), the anti-EGFR1 antibody and TGFβRII ECD fusion protein comprising SEQ ID Nos: 2 and 71, the anti-CD20 and TGFβRII ECD fusion protein comprising SEQ ID Nos: 3 and 72, the anti-VEGF antibody and TGFβRII ECD fusion protein comprising SEQ ID Nos: 4 and 73, the anti-CTLA-4 antibody and TGFβRII ECD fusion protein comprising SEQ ID Nos: 5 and 74. the anti-IL-2 Fc and TGFβRII ECD fusion protein comprising SEQ ID Nos: 6 and/or 7, the anti-CD25 antibody and TGFβRII ECD fusion protein comprising SEQ ID Nos: 8 and 75; the anti-CD25 (Basiliximab) and TGFβRII ECD fusion protein comprising SEQ Nos: 9 and 76: the PD-1 ectodomain, Fc and TGFβRII ECD fusion proteins comprising SEQ ID Nos: 11 and/or 12, the TGFβRII ectodomain, Fc and RANK ectodomain fusion proteins comprising SEQ ID Nos: 13 and/or 14, the anti-HER2/neu antibody and PD-1 ectodomain fusion protein comprising SEQ ID Nos: 15 and 70, the anti-EGFR1 antibody and PD-1 ectodomain fusion protein comprising SEQ ID Nos: 16 and 71, the anti-CD20 and PD-1 ectodomain fusion protein comprising SEQ ID Nos: 17 and 72, the anti-VEGF antibody and PD-1 ectodomain fusion protein comprising SEQ ID Nos: 18 and 73, the anti-CTLA-4 antibody and PD-1 ectodomain fusion protein comprising SEQ ID Nos: 19 and 74, the anti-CD25 antibody and PD-1 ectodomain fusion protein comprising SEQ ID Nos: 20 and 75; the anti-CD25 (Basiliximab) and PD-1 ectodomain fusion protein comprising SEQ ID Nos: 21 and 76; the IL-2, Fc and PD-1 ectodomain fusion proteins comprising SEQ ID NO: 16 and/or 23, the anti-CD4 antibody and PD-1 extracellular domain fusion protein comprising SEQ ID Nos: 24 and 77, the PD-1 ectodomain, Fc, RANK ECD fusion proteins comprising SEQ ID NO: 16 and/or 23, the anti-HER2/neu antibody and RANK ECD fusion protein comprising SEQ ID Nos: 27 and 70, the anti-EGFR1 antibody and RANK ECD fusion protein comprising SEQ ID Nos: 28 and 71, the anti-CD20 and RANK ECD fusion protein comprising SEQ ID Nos: 29 and 72, the anti-VEGF antibody and RANK ECD fusion protein comprising SEQ ID Nos: 30 and 73, the anti-CTLA-4 antibody and RANK ECD fusion protein comprising SEQ ID Nos: 31 and 74, the anti-CD25 antibody and RANK ECD fusion protein comprising SEQ ID Nos: 32 and 75; the anti-CD25 (Basiliximab) and RANK ECD fusion protein comprising SEQ ID Nos: 33 and 76, the IL-2, Fc and RANK ECD fusion proteins comprising SEQ ID NOs: 34 and/or 35, the anti-CD4 antibody and RANK ECD fusion protein comprising SEQ ID Nos: 36 and 77, the anti-TNFα antibody and PD-1 ligand 1 or PD-1 ligand 2 fusion proteins comprising SEQ ID Nos: 37 and 78, the TNFR2 extracellular biding domain, Fc and PD-1 ligand fusion protein comprising SEQ ID NO: 38 and/or 39, the anti-CD20 and PD-L1 fusion protein comprising SEQ ID Nos: 40 and 72, the anti-CD25 antibody and PD-L1 fusion protein comprising SEQ ID Nos: 41 and 75, the anti-CD25 (Basiliximab) and PD-1 cctodomain fusion protein comprising SEQ ID Nos: 42 and 76, the IL-2, Fc and PD-L1 fusion proteins comprising SEQ ID Nos: 43 and/or 44, the anti-CD4 antibody and PD-L1 fusion protein comprising SEQ ID Nos: 45 and 77, the CTLA-4 ECD, Fc (IgG Cγ1) and PD-L1 fusion proteins comprising SEQ ID Nos: 46 and/or 47, the TGF-β, Fc (IgG Cγ1) and PD-L1 fusion proteins comprising SEQ ID Nos: 48 and/or 49, the anti-TNF-α antibody and TGF-β fusion protein comprising SEQ ID Nos: 50 and 77, the TNFR2 extracellular binding domain, Fc and TGF-β fusion proteins comprising SEQ ID Nos: 51 and/or 52, the anti-CD20 and TGF-β fusion protein comprising SEQ ID Nos: 53 and 72, the anti-CD25 antibody and TGF-β fusion protein comprising SEQ ID Nos: 54 and 75, the anti-CD25 (Basiliximab) and TGF-β fusion protein comprising SEQ ID Nos: 55 and 76, the IL-2, Fc and TGF-β fusion proteins comprising SEQ ID Nos: 56 and/or 57, the CTLA-4 ECD, Fc (IgG Cγ1) arid TGF-β fusion proteins comprising SEQ ID Nos: 59 and/or 60, the anti-TNF-α antibody and RANK fusion protein comprising SEQ ID Nos: 61 and 78, the TNFR2 extracellular binding domain, Fc and RANK fusion proteins comprising SEQ ID Nos: 62 and/or 63, the CTLA-4 ECD, Fc (IgG Cγ1) and RANK fusion proteins comprising SEQ ID Nos: 64 and/or 65, the RANK, Fc, and TGF-β fusion proteins comprising SEQ ID Nos: 66 and/or 67, and the RANK, Fc, and PD-L1 fusion proteins comprising SEQ If) Nos: 68 and/or 69.

Anti-PD-L1 Antibodies

The anti-TIM-3 antibodies described herein can be administered in combination with any anti-PD-L1 antibody, or antigen-binding fragment thereof, described in the art. Anti-PD-L1 antibodies are commercially available, for example, the 29E2A3 antibody (Biolegend, Cat. No. 329701). Antibodies can be monoclonal, chimeric, humanized, or human. Antibody fragments include Fab, F(ab′)2, scFv and Fv fragments, which are described in further detail below.

Exemplary antibodies are described in PCT Publication WO 2013/079174, which describes avelumab. These antibodies can include a heavy chain variable region polypeptide including a CDR_{H1}, CDR_{H2}, and CDR_{H3} sequence, where:
(a) the CDR_{H1} sequence is X₁YX₂MX₃ (SEQ ID NO: 58);
(b) the CDR_{H2} sequence is SIYPSGGX₄TFYADX₅VKG (SEQ ID NO: 59);
(c) the CDR_{H3} sequence is IKLGTVTTVX₆Y (SEQ ID NO: 60);
   further where: X₁ is K, R, T, Q, G, A, W, M, I, or S; X₂ is V, R, K, L, M, or I; X₃ is H, T, N, Q, , V, Y, W, F, or M; X₄ is F or I; X₅ is S or T; X₆ is E or D.

In a one embodiment, X₁ is M, I, or S; X₂ is R, K, L, M, or I; X₃ is F or M; X₄ is F or I; X₅ is S or T; X₆ is E or D.

In another embodiment X₁ is M, I, or S; X₂ is L, M, or I; X₃ is F or M; X₄ is I; X₅ is S or T; X₆ is D.

In still another embodiment, X₁ is S; X₂ is I; X₃ is M; X₄ is I; X₅ is T; X₆ is D.

In another aspect, the polypeptide further includes variable region heavy chain framework (FR) sequences juxtaposed between the CDRs according to the formula: (HC-FR1)-(CDR_{H1})-(HC-FR2)-(CDR_{H2})-(HC-FR3)-(CDR_{H3})-(HC-FR4).

In yet another aspect, the framework sequences arc derived from human consensus framework sequences or human germline framework sequences.

In a still further aspect, at least one of the framework sequences is the following:

HC-FR1 is
(SEQ ID NO: 61)
EVQLLESGGGLVQPGGSLRLSCAASGFTFS;
HC-FR2 is
(SEQ ID NO: 62)
WVRQAPGKGLEWVS;
HC-FR3 is
(SEQ ID NO: 63)
RFTISRDNSKNTLYLQMNSLRAEDTAVYYCAR;
HC-FR4 is
(SEQ ID NO: 64)
WGQGTLVTVSS.

In a still further aspect, the heavy chain polypeptide is further combined with a variable region light chain including a CDR_{L1}, CDR_{L2}, and CDR_{L3}, where:
(a) the CDR_{L1} sequence is TGTX₇X₈DVGX₉YNYVS (SEQ ID NO: 65);
(b) the CDR_{L2} sequence is X₁₀VX₁₁X₁₂RPS (SEQ ID NO: 66);
(c) the CDR_{L3} sequence is SSX₁₃TX₁₄X₁₅X₁₆X₁₇RV (SEQ ID NO: 67);
   further where: X₇ is N or S; X₈ is T, R, or S; X₉ is A or G; X₁₀ is E or D; X₁₁ is I, N or S; X₁₂ is D, H or N; X₁₃ is F or Y; X₁₄ is N or S; X₁₅ is R, T or S; X₁₆ is G or S; X₁₇ is I or T.

In another embodiment, X₇ is N or S; X₈ is T, R, or S; X₉ is A or G; X₁₀ is E or D; is N or S; X₁₂ is N; X₁₃ is F or Y; X₁₄ is S; X₁₅ is S; X₁₆ is G or S; X₁₇ is T.

In still another embodiment, X₇ is S; X₈ is S; X₉ is G; X₁₀ is D; X₁₁ is S; X₁₂ is N; X₁₃ is Y; X₁₄ is S; X₁₅ is S; X₁₆ is S; X₁₇ is T.

In a still further aspect, the light chain further includes variable region light chain framework sequences juxtaposed between the CDRs according to the formula: (LC-CDR_{L1})-(LC-FR2)-(CDR_{L2})-(LC-FR3)-(CDR_{L3})-(LC-FR4).

In a still further aspect, the light chain framework sequences are derived from human consensus framework sequences or human germline framework sequences.

In a still further aspect, the light chain framework sequences are lambda light chain sequences.

In a still further aspect, at least one of the framework sequence is the following:

LC-FR1 is
(SEQ ID NO: 68)
QSALTQPASVSGSPGQSITISC;
LC-FR2 is
(SEQ ID NO: 69)
WYQQHPGKAPKLMIY;
LC-FR3 is
(SEQ ID NO: 70)
GVSNRFSGSKSGNTASLTISGLQAEDEADYYC;
LC-FR4 is
(SEQ ID NO: 71)
FGTGTKVTVL.

In another embodiment, the invention provides an anti-PD-L1 antibody or antigen binding fragment including a heavy chain and a light chain variable region sequence, where:
(a) the heavy chain includes a CDR_{H1}, CDR_{H2}, and CDR_{H3}, wherein further: (i) the CDR_{H1} sequence is X₁YX₂MX³ (SEQ ID NO: 72); (ii) the CDR_{H2} sequence is SIYPSGGX₄TFYADX₅VKG (SEQ ID NO: 73); (iii) the CDR_{H3} sequence is IKLGTVTTVX₆Y (SEQ ID NO: 74), and;
(b) the light chain includes a CDR_{L1}, CDR_{L2}, and CDR_{L3}, wherein further: (iv) the CDR_{L1} sequence is TGTX₇X₈DVGX₉YNYVS (SEQ ID NO: 75); (v) the CDR_{L2} sequence is X₁₀VX₁₁X₁₂RPS (SEQ ID NO: 76); (vi) the CDR_{L3} sequence is SSX₁₃TX₁₄X₁₅X₁₆X₁₇RV (SEQ ID NO: 77); wherein: X₁ is K, R, T, Q, G, A, W, M, I, or S; X₂ is V, R, K, L, M, or I; X₃ is H, T, N, Q, A, V, Y, W, F, or M; X₄ is F or I; X₅ is S or T; X₆ is E or D; X₇ is N or S; X₈ is T, R, or S; X₉ is A or G; X₁₀ is E or D; X₁₁ is I, N, or S; X₁₂ is D, H or N; X₁₃ is F or Y; X₁₄ is N or S; X₁₅ is R, T, or S; X₁₆ is G or S; X₁₇ is I or T.

In one embodiment, X₁ is M, I, or S; X₂ is R, K, L, M, or I; X₃ is F or M; X₄ is F or I; X₅ is S or T; X₆ is E or D; X₇ is N or S; X₈ is T, R, or S; X₉ is A or G; X₁₀ is E or D; X₁₁ is N or S; X₁₂ is N; X₁₃ is F or Y; X₁₄ is S; X₁₅ is S; X₁₆ is G or S; X₁₇ is T.

In another embodiment, X₁ is M, I, or S; X₂ is L, M, or I; X₃ is F or M; X₄ is I; X₅ is S or T; X₆ is D; X₇ is N or S; X₈ is T, R, or S; X₉ is A or G; X₁₀ is E or D; X₁₁ is N or S; X₁₂ is N; X₁₃ is F or Y; X₁₄ is S; X₁₅ is S; X₁₆ is G or S; X₁₇ is T.

In still another embodiment, X₁ is S; X₂ is I; X₃ is M; X₄ is I; X₅ is T; X₆ is D; X₇ is S; X₈ is S; X₉ is G; X₁₀ is D; X₁₁ is S; X₁₂ is N; X₁₃ is Y; X₁₄ is S; X₁₅ is S; X₁₆ is S; X₁₇ is T.

In a further aspect, the heavy chain variable region includes one or more framework sequences juxtaposed between the CDRs as: (HC-FR1)-(CDR_{H1})-(HC-FR2)-(CDR_{H2})-(HC-FR3)-(CDR_{H3})-(HC-FR4), and the light chain variable regions include one or more framework sequences juxtaposed between the CDRs as: (LC-FR1 MCDR_{L1})-(LC-FR2)-(CDR_{L2})-(LC-FR3)-(CDR_{L3})-(LC-FR4).

In a still further aspect, the framework sequences are derived from human consensus framework sequences or human germline sequences.

In a still further aspect, one or more of the heavy chain framework sequences is the following:

HC-FR1 is
(SEQ ID NO: 61)
EVQLLESGGGLVQPGGSLRLSCAASGFTFS;
HC-FR2 is
(SEQ ID NO: 62)
WVRQAPGKGLEWVS;
HC-FR3 is
(SEQ ID NO: 63)
RFTISRDNSKNTLYLQMNSLRAEDTAVYYCAR;
HC-FR4 is
(SEQ ID NO: 64)
WGQGTLVTVSS.

In a still further aspect, the light chain framework sequences are lambda light chain sequences.

In a still further aspect, one or more of the light chain framework sequences is the following:

LC-FR1 is
(SEQ ID NO: 68)
QSALTQPASVSGSPGQSITISC;
LC-FR2 is
(SEQ ID NO: 69)
WYQQHPGKAPKLMIY;
LC-FR3 is
(SEQ ID NO: 70)
GVSNRFSGSKSGNTASLTISGLQAEDEADYYC;
LC-FR4 is
(SEQ ID NO: 71)
FGTGTKVTVL.

In a still further aspect, the heavy chain variable region polypeptide, antibody, or antibody fragment further includes at least a C_{H}1 domain.

In a more specific aspect, the heavy chain variable region polypeptide, antibody, or antibody fragment further includes a C_{H}1, a C_{H}2, and a C_{H}3 domain.

In a still further aspect, the variable region light chain, antibody, or antibody fragment further includes a C_{L} domain.

In a still further aspect, the antibody further includes a C_{H}1, a C_{H}2, a C_{H}3, and a C_{L} domain.

In a still further specific aspect, the antibody further includes a human or murine constant region.

In a still further aspect, the human constant region is selected from the group consisting of IgG1, IgG2, IgG2, IgG3, IgG4.

In a still further specific aspect, the human or murine constant region is lgG1.

In yet another embodiment, the invention features an anti-PD-L1 antibody including a heavy chain and a light chain variable region sequence, where:
(a) the heavy chain includes a CDR_{H1}, a CDR_{H2}, and a CDR_{H3}, having at least 80% overall sequence identity to SYIMM (SEQ ID NO: 78), SIYPSGGITFYADTVKG (SEQ ID NO: 79), and IKLGTVTTVDY (SEQ ID NO: 80), respectively, and
(b) the light chain includes a CDR_{L1}, a CDR_{L2}, and a CDR_{L3}, having at least 80% overall sequence identity to TGTSSDVGGYNYVS (SEQ ID NO: 81), DVSNRPS (SEQ ID NO: 82), and SSYTSSSTRV (SEQ ID NO: 83), respectively.

In a specific aspect, the sequence identity is 81%, 82%, 83%, 84%, 85%, 86%, 87%, 88%, 89%, 90%, 91%, 92%, 93%, 94%, 95%, 96%, 97%, 98%, 99%, or 100%.

In yet another embodiment, the invention features an anti-PD-L1 antibody including a heavy chain and a light chain variable region sequence, where:
(a) the heavy chain includes a CDR_{H1}, a CDR_{H2}, and a CDR_{H3}, having at least 80% overall sequence identity to MYMMM (SEQ ID NO: 84), SIYPSGGITFYADSVKG (SEQ ID NO: 85), and IKLGTVTTVDY (SEQ ID NO: 80), respectively, and
(b) the light chain includes a CDR_{L1}, a CDR_{L2}, and a CDR_{L3}, having at least 80% overall sequence identity to TGTSSDVGAYNYVS (SEQ ID NO: 86), DVSNRPS (SEQ ID NO: 82), and SSYTSSSTRV (SEQ ID NO: 83), respectively.

In a specific aspect, the sequence identity is 81%, 82%, 83%, 84%, 85%, 86%, 87%, 88%, 89%, 90%, 91%, 92%, 93%, 94%, 95%, 96%, 97%, 98%, 99%, or 100%.

In a still further aspect, in the antibody or antibody fragment according to the invention, as compared to the sequences of CDR_{H1}, CDR_{H2}, and CDR_{H3}, at least those amino acids remain unchanged that are highlighted by underlining as follows:

(a) in CDR_{H1}
(SEQ ID NO: 78)
SYIMM,
(b) in CDR_{H2}
(SEQ ID NO: 79)
SIYPSGGITFYADTVKG,
(c) in CDR_{H3}
(SEQ ID NO: 80)
IKLGTVTTVDY;

and further where, as compared to the sequences of CDR_{L1}, CDR_{L2}, and CDR_{L3} at least those amino acids remain unchanged that are highlighted by underlining as follows:

(a) CDR_{L1} TGTSSDVGGYNYVS (SEQ ID NO: 81)

(b) CDR_{L2} DVSNRPS (SEQ ID NO: 82)

(c) CDR_{L3} SSYTSSSTRV (SEQ ID NO: 83).

In another aspect, the heavy chain variable region includes one or more framework sequences juxtaposed between the CDRs as: (HC-FR1)-(CDR_{H1})-(HC-FR2)-(CDR_{H2})-(HC-FR3)-(CDR_{H3})-(HC-FR4), and the light chain variable regions include one or more framework sequences juxtaposed between the CDRs as: (LC-FR1)-(CDR_{L1})-(LC-FR2)-(CDR_{L2})-(LC-FR3)-(CDR_{L3})-(LC-FR4).

In yet another aspect, the framework sequences are derived from human germline sequences.

In a still further aspect, one or more of the heavy chain framework sequences is the following:

HC-FR1 is
(SEQ ID NO: 61)
EVQLLESGGGLVQPGGSLRLSCAASGFTFS;
HC-FR2 is
(SEQ ID NO: 62)
WVRQAPGKGLEWVS;
HC-FR3 is
(SEQ ID NO: 63)
RFTISRDNSKNTLYLQMNSLRAEDTAVYYCAR;
HC-FR4 is
(SEQ ID NO: 64)
WGQGTLVTVSS.

In a still further aspect, the light chain framework sequences are derived from a lambda light chain sequence.

In a still further aspect, one or more of the light chain framework sequences is the following:

LC-FR1 is
(SEQ ID NO: 68)
QSALTQPASVSGSPGQSITISC;
LC-FR2 is
(SEQ ID NO: 69)
WYQQHPGKAPKLMIY;
LC-FR3 is
(SEQ ID NO: 70)
GVSNRFSGSKSGNTASLTISGLQAEDEADYYC;
LC-FR4 is
(SEQ ID NO: 71)
FGTGTKVTVL.

In a still further specific aspect, the antibody further includes a human or murine constant region.

In a still further aspect, the human constant region is selected from the group consisting of IgG1, IgG2, IgG2, IgG3, IgG4.

In a still further embodiment, the invention features an anti-PD-L1 antibody including a heavy chain and a light chain variable region sequence, where:

(a) the heavy chain sequence has at least 85%
sequence identity to the heavy chain sequence:
(SEQ ID NO: 87)
EVQLLESGGGLVQPGGSLRLSCAASGFTFSSYIMMVWRQAPGKGLEWVSS
IYPSGGITFYADWKGRFTISRDNSKNTLYLQMNSLRAEDTAVYYCARIKL
GTVTTVDYWGQGTLVTVSS,
and
(b) the light chain sequence has at least 85%
sequence identity to the light chain sequence:
(SEQ ID NO: 88)
QSALTQPASVSGSPGQSITISCTGTSSDVGGYNYVSWYQQHPGKAPKLMI
YDVSNRPSGVSNRFSGSKSGNTASLTISGLQAEDEADYYCSSYTSSSTRV
FGTGTKVTVL.

In a specific aspect, the sequence identity is 86%, 87%, 88%, 89%, 90%, 91%, 92%, 93%, 94%, 95%, 96%, 97%, 98%, 99%, or 100%.

In a still further embodiment, the invention provides for an anti-PD-L1 antibody including a heavy chain and a light chain variable region sequence, where:

(a) the heavy chain sequence has at least 85%
sequence identity to the heavy chain sequence:
(SEQ ID NO: 89)
EVQLLESGGGLVQPGGSLRLSCAASGETFSMYMMMWVRQAPGKGLEVWS
SIYPSGGITFYADSVKGRFTISRDNSKNTLYLQMNSLRAEDTAIYYCAR
IKLGTVTTVDYWGQGTLVTVSS,
and
(b) the light chain sequence has at least 85%
sequence identity to the light chain sequence:
(SEQ ID NO: 90)
QSALTQPASVSGSPGQSMSCTGTSSDVGAYNYVSWYQQHPGKAPKLMIY
DVSNRPSGVSNRFSGSKSGNTASLTISGLQAEDEADYYCSSYTSSSTRV
FGTGTKVTVL.

In a specific aspect, the sequence identity is 86%, 87%, 88%, 89%, 90%, 91%, 92%, 93%, 94%, 95%, 96%, 97%, 98%, 99% or 100%.

Anti PD-L1/TGFβ Trap Fusion Proteins

The anti-TIM-3 antibodies described herein can be administered in combination with any anti-PD-L1/TGFβ Trap known in the art. Anti-PD-L1/TGFβ Trap refers to an anti-PD-L1 antibody-TGFβ Receptor II extracellular domain (ECD) fusion protein.

In one embodiment, the Anti-PD-L1/TGFβ Trap comprises an anti-PD-L1 antibody as described herein (for example, in the above section entitled “Anti-PD-L1 Antibodies”).

In one embodiment, the anti-PD-L1/TGFβ Trap is a protein having the amino acid sequence of bintrafusp alfa, as described in International Patent Publication WO 2015/118175 and as reflected by the amino acid sequence given by CAS Registry Number 1918149-01-5. Bintrafusp alfa comprises a light chain that is identical to the light chain of an anti-PD-L1 antibody (SEQ ID NO: 91). Bintrafusp alfa further comprises a fusion polypeptide having the sequence corresponding SEQ ID NO: 93, composed of the heavy chain of an anti-PD-L1 antibody (SEQ ID NO: 92), wherein the C-terminal lysine residue of heavy chain was mutated to alanine, genetically fused to via a flexible (Gly₄Ser)₄Gly linker (SEQ ID NO: 97) to the N-terminus of the soluble TGFβ Receptor II (SEQ ID NO: 96). Bintrafusp alfa is encoded by SEQ ID NO: 94 (DNA encoding the anti-PD-L1 light chain) and SEQ ID NO: 95 (DNA encoding the anti-PD-L1/TGFβ Receptor II).

In one embodiment, the anti-PD-L1/TGFβ Trap is bintrafusp alfa, a protein having the amino acid sequence of bintrafusp alpha and also a glycosylation form that results from the protein being produced in CHO cells, wherein the heavy chain is glycosylated at Asn-300, Asn-518, Asn-542, and Asn-602 (i.e., of SEQ ID NO: 93). (See, WHO Drug Information, Vol. 32, No. 2, 2018, p. 293.)

Peptide sequence of the secreted LC of anti-PD-L1

(SEQ ID NO: 91)
QSALTQPASVSGSPGQSITISCTGTSSDVGGYNYVSWYQQHPGKAPKLM
IYDVSNRPSGVSNRFSGSKSGNTASLTISGLQAEDEADYYCSSYTSSST
RVFGTGTKVTVLGQPKANPTVTLFPPSSEELQANKATLVCLISDFYPGA
VTVAWKADGSPVKAGVETTKPSKQSNNKYAASSYLSLTPEQWKSHRSYS
CQVTHEGSTVEKTVAPTECS

Peptide sequence of the secreted H chain of anti-PDL1

(SEQ ID NO: 92)
EVQLLESGGGLVQPGGSLRLSCAASGFTFSSYIMMWVRQAPGKGLEWVS
SIYPSGGITFYADTVKGRFTISRDNSKNTLYLQMNSLRAEDTAVYYCAR
IKLGTVTTVDYWGQGTLVTVSSASTKGPSVFPLAPSSKSTSGGTAALGC
LVKDYFPEPVTVSWNSGALTSGVHTFPAVLQSSGLYSLSSVVTVPSSSL
GTQTYICNVNHKPSNTKVDKRVEPKSCDKTHTCPPCPAPELLGGPSVFL
FPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAK
GQPREPQVYTLPPSREEMTKNQVSLTCLVKGFYPSDIAVEWESNGQPEN
NYKTTPPVLDSDGSFFLYSKLTVDKSRWQQGNVFSCSVMHEALHNHYTQ
KSLSLSPGK

Peptide sequence of the secreted H chain of anti-PDL1/TGFβ Trap

(SEQ ID NO: 93)
EVQLLESGGGLVQPGGSLRLSCAASGFTFSSYIMMWVRQAPGKGLEWVS
SIYPSGGITFYADTVKGRFTISRDNSKNTLYLQMNSLRAEDTAVYYCAR
IKLGTVTIVDYWGQGTLVTVSSASTKGPSVFPLAPSSKSTSGGTAALGC
LVKDYFPEPVTVSWNSGALTSGVHTFPAVLQSSGLYSLSSVVTVPSSSL
GTQTYICNVNHKPSNTKVDKRVEPKSCDKTHTCPPCPAPELLGGPSVFL
FPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAK
GQPREPQVYTLPPSREEMTKNQVSLTCLVKGFYPSDIAVEWESNGQPEN
NYKTTPPVLDSDGSFFLYSKLTVDKSRWQQGNVFSCSVMHEALHNHYTQ
KSLSLSPGAGGGGSGGGGSGGGGSGGGGSGIPPHVQKSVNNDMIVTDNN
GAVKFPQLCKFCDVRFSTCDNQKSCMSNCSITSICEKPQEVCVAVWRKN
DENITLETVCHDPKLPYHDFILEDAASPKCIMKEKKKPGETFFMCSCSS
DECNDNIIFSEEYNTSNPD

DNA sequence from the translation initiation codon to the translation stop codon of the anti-PD-L1 lambda light chain (the leader sequence preceding the VL is the signal peptide from urokinase plasminogen activator)

(SEQ ID NO: 94)
atgagggccctgctggctagactgctgctgtgcgtgctggtcgtgtccg
acagcaagggcCAGTCCGCCCTGACCCAGCCTGCCTCCGTGTCTGGCTC
CCCTGGCCAGTCCATCACCATCAGCTGCACCGGCACCTCCAGCGACGTG
GGCGGCTACAACTACGTGTCCTGGTATCAGCAGCACCCCGGCAAGGCCC
CCAAGCTGATGATCTACGACGTGTCCAACCGGCCCTCCGGCGTGTCCAA
CAGATTCTCCGGCTCCAAGTCCGGCAACACCGCCTCCCTGACCATCAGC
GGACTGCAGGCAGAGGACGAGGCCGACTACTACTGCTCCTCCTACACCT
CCTCCAGCACCAGAGTGTTCGGCACCGGCACAAAAGTGACCGTGCTGgg
ccagcccaaggccaacccaaccgtgacactgttccccccatcctccgag
gaactgcaggccaacaaggccaccctggtctgcctgatctcagatttct
atccaggcgccgtgaccgtggcctggaaggctgatggctccccagtgaa
ggccggcgtggaaaccaccaagccctccaagcagtccaacaacaaatac
gccgcctcctcctacctgtccctgacccccgagcagtggaagtcccacc
ggtcctacagctgccaggtcacacacgagggctccaccgtggaaaagac
cgtcgcccccaccgagtgctcaTGA

DNA sequence from the translation initiation codon to the translation stop codon (mVK SP leader: small underlined; VH: capitals; IgG1m3 with K to A mutation: small letters; (G4S)x4-G linker: bold capital letters; TGFβRII: bold underlined small letters; two stop codons: bold underlined capital letters)

(SEQ ID NO: 95)
atggaaacagacaccctgctgctgtgggtgctgctgctgtgggtgcccg
gctccacaggcGAGGTGCAGCTGCTGGAATCCGGCGGAGGACTGGTGCA
GCCTGGCGGCTCCCTGAGACTGTCTTGCGCCGCCTCCGGCTTCACCTTC
TCCAGCTACATCATGATGTGGGTGCGACAGGCCCCTGGCAAGGGCCTGG
AATGGGTGTCCTCCATCTACCCCTCCGGCGGCATCACCTTCTACGCCGA
CACCGTGAAGGGCCGGTTCACCATCTCCCGGGACAACTCCAAGAACACC
CTGTACCTGCAGATGAACTCCCTGCGGGCCGAGGACACCGCCGTGTACT
ACTGCGCCCGGATCAAGCTGGGCACCGTGACCACCGTGGACTACTGGGG
CCAGGGCACCCTGGTGACAGTGTCCTCCgctagcaccaagggcccatcg
gtcttccccctggcaccctcctccaagagcacctctgggggcacagcgg
ccctgggctgcctggtcaaggactacttccccgaaccggtgacggtgtc
gtggaactcaggcgccctgaccagcggcgtgcacaccttcccggctgtc
ctacagtcctcaggactctactccctcagcagcgtggtgaccgtgccct
ccagcagcttgggcacccagacctacatctgcaacgtgaatcacaagcc
cagcaacaccaaggtggacaagagagttgagcccaaatcttgtgacaaa
actcacacatgcccaccgtgcccagcacctgaactcctggggggaccgt
cagtcttcctcttccccccaaaacccaaggacaccctcatgatctcccg
gacccctgaggtcacatgcgtggtggtggacgtgagccacgaagaccct
gaggtcaagttcaactggtacgtggacggcgtggaggtgcataatgcca
agacaaagccgcgggaggagcagtacaacagcacgtaccgtgtggtcag
cgtcctcaccgtcctgcaccaggactggctgaatggcaaggagtacaag
tgcaaggtctccaacaaagccctcccagcccccatcgagaaaaccatct
ccaaagccaaagggcagccccgagaaccacaggtgtacaccctgccccc
atcccgggaggagatgaccaagaaccaggtcagcctgacctgcctggtc
aaaggcttctatcccagcgacatcgccgtggagtgggagagcaatgggc
agccggagaacaactacaagaccacgcctcccgtgctggactccgacgg
ctccttcttcctctatagcaagctcaccgtggacaagagcaggtggcag
caggggaacgtcttctcatgctccgtgatgcatgaggctctgcacaacc
actacacgcagaagagcctctccctgtccccgggtgctGGCGGCGGAGG
AAGCGGAGGAGGTGGCAGCGGTGGCGGTGGCTCCGGCGGAGGTGGCTCC
GGAatccctccccacgtgcagaagtccgtgaacaacgacatgatcgtga
ccgacaacaacggcgccgtgaagttccctcagctgtgcaagttctgcga
cgtgaggttcagcacctgcgacaaccagaagtcctgcatgagcaactgc
agcatcacaagcatctgcgagaagccccaggaggtgtgtgtggccgtgt
ggaggaagaacgacgaaaacatcaccctcgagaccgtgtgccatgaccc
caagctgccctaccacgacttcatcctggaagacgccgcctcccccaag
tgcatcatgaaggagaagaagaagcccggcgagaccttcttcatgtgca
gctgcagcagcgacgagtgcaatgacaacatcatctttagcgaggagta
caacaccagcaaccccgacTGATAA

### A Human TGFβRII Isoform B Extracellular Domain Polypeptide

(SEQ ID NO: 96)
IPPHVQKSVNNDMIVTDNNGAVKFPQLCKFCDVRFSTCDNQKSCMSNCS
ISICEKPQEVCVAVWRKNDENITLETVCHDPKLPYHDFILEDAASPKCI
MKEKKKPGETFFMCSCSSDECNDNIIFSEEYNTSNPD
(Gly₄Ser)₄Gly linker
(SEQ ID NO: 97)
GGGGSGGGGSGGGGSGGGGSG

Anti-PD-L1/TGFβ Trap molecules useful in the present invention may comprise sequences having at least 85%, at least 90%, at least 95%, at least 96%, at least 97%, at least 98%, or at least 99% sequence identity to any one of SEQ ID NOs: 91-96, as described above.

In some embodiments, the anti-PD-L1/TGFβ Trap is an anti-PD-L1/TGFβ Trap molecule disclosed in WO 2018/205985. For example, the anti-PD-L1/TGFβ Trap is one of the constructs listed in Table 2 of WO 2018/205985, such as construct 9 or 15 thereof.

In other embodiments, anti-PD-L1/TGFβ Trap is a heterotetramer, consisting of two polypeptides each having the light chain sequence corresponding to SEQ ID NO: 12 of WO 2018/205985 and two fusion polypeptides each having the heavy chain sequence corresponding to SEQ ID NO: 11 of WO 2018/205985 fused via a linker sequence (G₄S)ₓG (wherein x can be 4 or 5) (SEQ ID NO: 117) to the TGFβRII extracellular domain sequence corresponding to SEQ ID NO: 14 (wherein “x” of the linker sequence is 4) or SEQ ID NO: 15 (wherein “x” of the linker sequence is 5) of WO 2018/205985.

In certain embodiments, an anti-PD-L1/TGFβ Trap molecule includes a first and a second polypeptide. The first polypeptide includes: (a) at least a variable region of a heavy chain of an antibody that binds to human protein Programmed Death Ligand 1 (PD-L1); and (b) human Transforming Growth Factor β Receptor II (TGFβRII), or a fragment thereof, capable of binding Transforming Growth Factor β (TGFβ) (e.g., a soluble fragment). The second polypeptide includes at least a variable region of a light chain of an antibody that binds PD-L1, in which the heavy chain of the first polypeptide and the light chain of the second polypeptide, when combined, form an antigen binding site that binds PD-L1 (e.g., any of the antibodies or antibody fragments described herein). In certain embodiments, the anti-PD-L1/TGFβ Trap molecule is a heterotetramer, comprising the two immunoglobulin light chains of anti-PD-L1, and two heavy chains comprising the heavy chain of anti-PD-L1 genetically fused via a flexible glycine-serine linker (e.g., (G₄S)ₓG, wherein x can be 4 or 5 (SEQ ID NO: 117)) to the extracellular domain of the human TGFβRII.

A Truncated Human TGFβRII Isoform B Extracellular
Domain Polypeptide
SEQ ID NO: 104
GAVKFPQLCKFCDVRFSTCDNQKSCMSNCSITSICEKPQEVCVAVWRKN
DENITLETVCHDPKLPYHDFILEDAASPKCIMKEKKKPGETFFMCSCSS
DECNDNIIFSEEYNTSNPD (identical to SEQ ID NO: 14
in WO 2018/205985)
A Truncated Human TGFβRII Isoform B Extracellular
Domain Polypeptide
SEQ ID NO: 105
VKFPQLCKFCDVRFSTCDNQKSCMSNCSITSICEKPQEVCVAVWRKNDE
NITLETVCHDPKLPYHDFILEDAASPKCIMKEKKKPGETFFMCSCSSDE
CNDNIIFSEEYNTSNPD (identical to SEQ ID NO: 15
in WO 2018/205985)
A Truncated Human TGFβRII Isofonn B Extracellular
Domain Polypeptide
SEQ ID NO: 106
VTDNNGAVKFPQLCKFCDVRFSTCDNQKSCMSNCSITSICEKPQEVCVA
VWRKNDENITLETVCHDPKLPYHDFILEDAASPKCIMKEKKKPGETFFM
CSCSSDECNDNIIFSEEYNTSNPD
A Truncated Human TGFβRII Isoform B Extracellular
Domain Polypeptide
SEQ ID NO: 107
LCKFCDVRFSTCDNQKSCMSNCSITSICEKPQEVCVAVWRKNDENITLE
TVCHDPKLPYHDFILEDAASPKCIMKEKKKPGETFFMCSCSSDECNDNI
IFSEEYNTSNPD
A Mutated Human TGFβRII Isoform B Extracellular
Domain Polypeptide
SEQ ID NO: 108
VTDNAGAVKFPQLCKFCDVRFSTCDNQKSCMSNCSITSICEKPQEVCVA
VWRKNDENITLETVCHDPKLPYHDFILEDAASPKCIMKEKKKPGETFFM
CSCSSDECNDNIIFSEEYNTSNPD
SEQ ID NO: 109
Polypeptide sequence of the heavy chain variable
region of anti-PD-L1 antibody
QVQLQESGPGLVKPSQTLSLICTVSGGSISNDYWTWIRQHPGKGLEYIG
YISYTGSTYYNPSLKSRVTISRDTSKNQFSLKLSSVTAADTAVYYCARS
GGWLAPFDYWGRGTLVTVSS
Polypeptide sequence of the light chain variable
region of anti-PD-L1 antibody
SEQ ID NO: 110
DIVMTQSPDSLAVSLGERATINCKSSQSLFYHSNQKHSLAWYQQKPGQP
PKLLIYGASTRESGVPDRFSGSGSGTDFTLTISSLQAEDVAVYYCQQYY
GYPYTFGGGTKVEIK
Polypeptide sequence of the heavy chain variable
region of anti-PD-L1 antibody
SEQ ID NO: 111
QVQLVQSGAEVKKPGASVKVSCKASGYTFTSYWMHWVRQAPGQGLEWMG
RIGPNSGFTSYNEKFKNRVTMTRDTSTSTVYMELSSLRSEDTAVYYCAR
GGSSYDYFDYWGQGTTVTVSS
Polypeptide sequence of the light chain variable
region of anti-PD-L1 antibody
SEQ ID NO: 112
DIVLTQSPASLAVSPGQRATITCRASESVSIHGTHLMHWYQQKPGQPPK
LLIYAASNLESGVPARFSGSGSGTDFTLTINPVEAEDTANYYCQQSFED
PLTFGQGTKLEIK
Polypeptide sequence of the heavy chain of anti-
PD-L1 antibody
SEQ ID NO: 113
QVQLQESGPGLVKPSQTLSLTCTVSGGSISNDYWTWIRQHPGKGLEYIG
YISYTGSTYYNPSLKSRVTISRDTSKNQFSLKLSSVTAADTAVYYCARS
GGWLAPFDYWGRGTLVTVSSASTKGPSVFPLAPCSRSTSESTAALGCLV
KDYFPEPVTVSWNSGALTSGVHTFPAVLQSSGLYSLSSVVTVPSSSLGT
KTYTCNVDHKPSNTKVDKRVESKYGPPCPPCPAPEAAGGPSVFLFPPKP
KDTLMISRTPEVTCVVVDVSQEDPEVQFNWYVDGVEVHNAKTKPREEQF
NSTYRVVSVLTVLHQDWLNGKEYKCKVSNKGLPSSIEKTISKAKGQPRE
PQVYTLPPSQEEMTKNQVSLTCLVKGFYPSDIAVEWESNGQPENNYKTT
PPVLDSDGSFFLYSRLTVDKSRWQEGNVFSCSVMHEALHNHYTQKSLSL
SLGK
SEQ ID NO: 114
Polypeptide sequence of the light chain of anti-
PD-L1 antibody
DIVMTQSPDSLAVSLGERATINCKSSQSLFYHSNQKHSLAWYQQKPGQP
PKLLIYGASTRESGVPDRFSGSGSGTDFTLTISSLQAEDVAVYYCQQYY
GYPYTFGGGTKVEIKRTVAAPSVFIFPPSDEQLKSGTASVVCLLNNFYP
REAKVQWKVDNALQSGNSQESVTEQDSKDSTYSLSSTLTLSKADYEKHK
VYACEVTHQGLSSPVTKSFNRGEC
Polypeptide sequence of the heavy chain of anti-
PD-L1 antibody
SEQ ID NO: 115
QVQLVQSGAEVKKPGASVKVSCKASGYTFTSYWMHWVRQAPGQGLEWMG
RIGPNSGFTSYNEKFKNRVTMTRDTSTSTVYMELSSLRSEDTAVYYCAR
GGSSYDYFDYWGQGTTVTVSSASTKGPSVFPLAPCSRSTSESTAALGCL
VKDYFPEPVTVSWNSGALTSGVHTFPAVLQSSGLYSLSSVVTVPSSSLG
TKTYTCNVDHKPSNTKVDKRVESKYGPPCPPCPAPEAAGGPSVFLFPPK
PKDTLMISRTPEVTCVVVDVSQEDPEVQFNWYVDGVEVHNAKTKPREEQ
FNSTYRVVSVLTVLHQDWLNGKEYKCKVSNKGLPSSIEKTISKAKGQPR
EPQVYTLPPSQEEMTKNQVSLTCLVKGFYPSDIAVEWESNGQPENNYKT
TPPVLDSDGSFFLYSRLTVDKSRWQEGNVFSCSVMHEALHNHYTQKSLS
LSLGA (identical to SEQ ID NO: 11 of WO 2018/
205985)
SEQ ID NO: 116
Polypeptide sequence of the light chain of anti-
PD-L1 antibody
DIVLTQSPASLAVSPGQRATITCRASESVSIHGTHLMHWYQQKPGQPPK
LLIYAASNLESGVPARFSGSGSGTDFTLTINPVEAEDTANYYCQQSFED
PLTFGQGTKLEIKRTVAAPSVFIFPPSDEQLKSGTASVVCLLNNFYPRE
AKVQWKVDNALQSGNSQESVTEQDSKDSTYSLSSTLTLSKADYEKHKVY
ACEVTHQGLSSPVTKSFNRGEC (identical to SEQ ID
NO: 12 of WO 2018/205985)

Anti-PD-L1/TGFβ Trap molecules useful in the present invention may comprise sequences having at least 85%, at least 90%, at least 95%, at least 96%, at least 97%, at least 98%, or at least 99% sequence identity to any one of SEQ ID NOs: 104-116, as described above.

### Methods of Treatment

As used herein, “treat,” “treating,” and “treatment” mean the treatment of a disease in a mammal, e.g., in a human. This includes: (a) inhibiting the disease, i.e., arresting its development; and (b) relieving the disease, i.e., causing regression of the disease state.

Generally, a therapeutically effective amount of anti-TIM-3 antibody or another therapeutic agent described herein (alone or in combination with another treatment, e.g., a second therapeutic agent) is in the range of 0.1 mg/kg to 100 mg/kg, e.g., 1 mg/kg to 100 mg/kg, e.g., 1 mg/kg to 10 mg/kg. In certain embodiments, a therapeutically effective amount of an anti-TIM-3 antibody or another therapeutic agent described herein can be administered at a dose from about 0.1 to about 1 mg/kg, from about 0.1 to about 5 mg/kg, from about 0.1 to about 10 mg/kg, from about 0.1 to about 25 mg/kg, from about 0.1 to about 50 mg/kg, from about 0.1 to about 75 mg/kg, from about 0.1 to about 100 mg/kg, from about 0.5 to about 1 mg/kg, from about 0.5 to about 5 mg/kg, from about 0.5 to about 10 mg/kg, from about 0.5 to about 25 mg/kg, from about 0.5 to about 50 mg/kg, from about 0.5 to about 75 mg/kg, from about 0.5 to about 100 mg/kg, from about 1 to about 5 mg/kg, from about 1 to about 10 mg/kg, from about 1 to about 25 mg/kg, from about 1 to about 50 mg/kg, from about 1 to about 75 mg/kg, from about 1 to about 100 mg/kg, from about 5 to about 10 mg/kg, from about 5 to about 25 mg/kg, from about 5 to about 50 mg/kg, from about 5 to about 75 mg/kg, from about 5 to about 100 mg/kg, from about 10 to about 25 mg/kg, from about 10 to about 50 mg/kg, from about 10 to about 75 mg/kg, from about 10 to about 100 mg/kg, from about 25 to about 50 mg/kg, from about 25 to about 75 mg/kg, from about 25 to about 100 mg/kg, from about 50 to about 75 mg/kg, from about 50 to about 100 mg/kg, from about 75 to about 100 mg/kg. The amount administered will depend on variables such as the type and extent of disease or indication to be treated, the overall health of the patient, the in vivo potency of the antibody, the pharmaceutical formulation, and the route of administration. The initial dosage can be increased beyond the upper level in order to rapidly achieve the desired blood-level or tissue level. Alternatively, the initial dosage can be smaller than the optimum, and the dosage may be progressively increased during the course of treatment. Human dosage can be optimized, e.g., in a conventional Phase I dose escalation study designed to run from 0.5 mg/kg to 30 mg/kg.

In certain embodiments, the anti-TIM-3 antibody or another therapeutic agent described herein (alone or in combination with another treatment, e.g., a second therapeutic agent) can be administered as a flat (fixed) dose (rather than in proportion to a mammal's body weight, i.e., a mg/kg dosage). A therapeutically effective amount of an anti-TIM-3 antibody can be a flat (fixed) dose of about 5 mg to about 3500 mg. For example, the dose can be from about 5 to about 250 mg, from about 5 to about 500 mg, from about 5 to about 750 mg, from about 5 to about 1000 mg, from about 5 to about 1250 mg, from about 5 to about 1500 mg, from about 5 to about 1750 mg, from about 5 to about 2000 mg, from about 5 to about 2250 mg, from about 5 to about 2500 mg, from about 5 to about 2750 mg, from about 5 to about 3000 mg, from about 5 to about 3250 mg, from about 5 to about 3500 mg, from about 250 to about 500 mg, from about 250 to about 750 mg, from about 250 to about 1000 mg, from about 250 to about 1250 mg, from about 250 to about 1500 mg, from about 250 to about 1750 mg, from about 250 to about 2000 mg, from about 250 to about 2250 mg, from about 250 to about 2500 mg, from about 250 to about 2750 mg, from about 250 to about 3000 mg, from about 250 to about 3250 mg, from about 250 to about 3500 mg, from about 500 to about 750 mg, from about 500 to about 1000 mg, from about 500 to about 1250 mg, from about 500 to about 1500 mg, from about 500 to about 1750 mg, from about 500 to about 2000 mg, from about 500 to about 2250 mg, from about 500 to about 2500 mg, from about 500 to about 2750 mg, from about 500 to about 3000 mg, from about 500 to about 3250 mg, from about 500 to about 3500 mg, from about 750 to about 1000 mg, from about 750 to about 1250 mg, from about 750 to about 1500 mg, from about 750 to about 1750 mg, from about 750 to about 2000 mg, from about 750 to about 2250 mg, from about 750 to about 2500 mg, from about 750 to about 2750 mg, from about 750 to about 3000 mg, from about 750 to about 3250 mg, from about 750 to about 3500 mg, from about 1000 to about 1250 mg, from about 1000 to about 1500 mg, from about 1000 to about 1750 mg, from about 1000 to about 2000 mg, from about 1000 to about 2250 mg, from about 1000 to about 2500 mg, from about 1000 to about 2750 mg, from about 1000 to about 3000 mg, from about 1000 to about 3250 mg, from about 1000 to about 3500 mg, from about 1250 to about 1500 mg, from about 1250 to about 1750 mg, from about 1250 to about 2000 mg, from about 1250 to about 2250 mg, from about 1250 to about 2500 mg, from about 1250 to about 2750 mg, from about 1250 to about 3000 mg, from about 1250 to about 3250 mg, from about 1250 to about 3500 mg, from about 1500 to about 1750 mg, from about 1500 to about 2000 mg, from about 1500 to about 2250 mg, from about 1500 to about 2500 mg, from about 1500 to about 2750 mg, from about 1500 to about 3000 mg, from about 1500 to about 3250 mg, from about 1500 to about 3500 mg, from about 1750 to about 2000 mg, from about 1750 to about 2250 mg, from about 1750 to about 2500 mg, from about 1750 to about 2750 mg, from about 1750 to about 3000 mg, from about 1750 to about 3250 mg, from about 1750 to about 3500 mg, from about 2000 to about 2250 mg, from about 2000 to about 2500 mg, from about 2000 to about 2750 mg, from about 2000 to about 3000 mg, from about 2000 to about 3250 mg, from about 2000 to about 3500 mg, from about 2250 to about 2500 mg, from about 2250 to about 2750 mg, from about 2250 to about 3000 mg, from about 2250 to about 3250 mg, from about 2250 to about 3500 mg, from about 2500 to about 2750 mg, from about 2500 to about 3000 mg, from about 2500 to about 3250 mg, from about 2500 to about 3500 mg, from about 2750 to about 3000 mg, from about 2750 to about 3250 mg, from about 2750 to about 3500 mg, from about 3000 to about 3250 mg, from about 3000 to about 3500 mg, or from about 3250 to about 3500 mg. Human dosage can be optimized, e.g., in a conventional Phase I dose escalation study designed to run from a flat (fixed) dose of 5 mg to 3200 mg.

Dosing frequency can vary, depending on factors such as route of administration, dosage amount, scrum half-life of the antibody, and the disease being treated. Exemplary dosing frequencies are once per week, once every two weeks, once every three weeks and once every four weeks. In some embodiments, dosing is once every two weeks. A preferred route of administration is parenteral, e.g., intravenous infusion. Formulation of monoclonal antibody-based drugs is within ordinary skill in the art. In some embodiments, the antibody is lyophilized, and then reconstituted in buffered saline, at the time of administration.

For therapeutic use, an antibody preferably is combined with a pharmaceutically acceptable carrier. As used herein, “pharmaceutically acceptable carrier” means buffers, carriers, and excipients suitable for use in contact with the tissues of human beings and animals without excessive toxicity, irritation, allergic response, or other problem or complication, commensurate with a reasonable benefit/risk ratio. The carrier(s) should be “acceptable” in the sense of being compatible with the other ingredients of the formulations and not deleterious to the recipient. Pharmaceutically acceptable carriers include buffers, solvents, dispersion media, coatings, isotonic and absorption delaying agents, and the like, that are compatible with pharmaceutical administration. The use of such media and agents for pharmaceutically active substances is known in the art.

Pharmaceutical compositions containing antibodies, such as those disclosed herein, can be presented in a dosage unit form and can be prepared by any suitable method. A pharmaceutical composition should be formulated to be compatible with its intended route of administration. Examples of routes of administration are intravenous (IV), intradermal, inhalation, transdermal, topical, transmucosal, and rectal administration. A preferred route of administration for monoclonal antibodies is IV infusion. Useful formulations can be prepared by methods well known in the pharmaceutical art. For example, see Remington's Pharmaceutical Sciences, 18th ed. (Mack Publishing Company, 1990). Formulation components suitable for parenteral administration include a sterile diluent such as water for injection, saline solution, fixed oils, polyethylene glycols, glycerine, propylene glycol or other synthetic solvents; antibacterial agents such as benzyl alcohol or methyl paraben; antioxidants such as ascorbic acid or sodium bisulfite; chelating agents such as EDTA; buffers such as acetates, citrates or phosphates; and agents for the adjustment of tonicity such as sodium chloride or dextrose.

For intravenous administration, suitable carriers include physiological saline, bacteriostatic water, Cremophor ELTM (BASF, Parsippany, N.J.) or phosphate buffered saline (PBS). The carrier should be stable under the conditions of manufacture and storage, and should be preserved against microorganisms. The carrier can be a solvent or dispersion medium containing, for example, water, ethanol, polyol (for example, glycerol, propylene glycol, and liquid polyethylene glycol), and suitable mixtures thereof.

Pharmaceutical formulations preferably are sterile. Sterilization can be accomplished, for example, by filtration through sterile filtration membranes. Where the composition is lyophilized, filter sterilization can be conducted prior to or following lyophilization and reconstitution.

The intravenous drug delivery formulation of the present disclosure for use in a method of treating cancer or inhibiting tumor growth in a mammal may be contained in a bag, a pen, or a syringe. In certain embodiments, the bag may be connected to a channel comprising a tube and/or a needle. In certain embodiments, the formulation may be a lyophilized formulation or a liquid formulation. In certain embodiments, the formulation may be freeze-dried (lyophilized) and contained. In certain embodiments, the about 40 mg-about 100 mg of freeze-dried formulation may be contained in one vial. In certain embodiments, the formulation may be a liquid formulation of a protein product that includes an anti-TIM-3 antibody as described herein and stored as about 250 mg/vial to about 2000 mg/vial.

Liquid Formulation

This disclosure provides a liquid aqueous pharmaceutical formulation including a therapeutically effective amount of the protein of the present disclosure (e.g., anti-TIM-3 antibody) in a buffered solution forming a formulation for use in a method of treating cancer or inhibiting tumor growth in a mammal.

These compositions for use in a method of treating cancer or inhibiting tumor growth in a mammal may be sterilized by conventional sterilization techniques, or may be sterile filtered. The resulting aqueous solutions may be packaged for use as-is, or lyophilized, the lyophilized preparation being combined with a sterile aqueous carrier prior to administration. The pH of the preparations typically will be between 3 and 11, more preferably between 5 and 9 or between 6 and 8, and most preferably between 7 and 8, such as 7 to 7.5. The resulting compositions in solid form may be packaged in multiple single dose units, each containing a fixed amount of the above-mentioned agent or agents. The composition in solid form can also be packaged in a container for a flexible quantity.

In certain embodiments, the present disclosure provides for use in a method of treating cancer or inhibiting tumor growth in a mammal, a formulation with an extended shelf life including a protein of the present disclosure (e.g., an anti-TIM-3 antibody), in combination with mannitol, citric acid monohydrate, sodium citrate, disodium phosphate dihydrate, sodium dihydrogen phosphate dihydrate, sodium chloride, polysorbate 80, water, and sodium hydroxide.

In certain embodiments, an aqueous formulation for use in a method of treating cancer or inhibiting tumor growth in a mammal is prepared including a protein of the present disclosure (e.g., an anti-TIM-3 antibody) in a pH-buffered solution. The buffer of this invention may have a pH ranging from about 4 to about 8, e.g., from about 4 to about 8, from about 4.5 to about 8, from about 5 to about 8, from about 5.5 to about 8, from about 6 to about 8, from about 6.5 to about 8, from about 7 to about 8, from about 7.5 to about 8, from about 4 to about 7.5, from about 4.5 to about 7.5, from about 5 to about 7.5, from about 5.5 to about 7.5, from about 6 to about 7.5, from about 6.5 to about 7.5, from about 4 to about 7, from about 4.5 to about 7, from about 5 to about 7, from about 5.5 to about 7, from about 6 to about 7, from about 4 to about 6.5, from about 4.5 to about 6.5, from about 5 to about 6.5, from about 5.5 to about 6.5, from about 4 to about 6.0, from about 4.5 to about 6.0, from about 5 to about 6, or from about 4.8 to about 5.5, or may have a pH of about 5.0 to about 5.2. Ranges intermediate to the above recited pH's are also intended to be part of this disclosure. For example, ranges of values using a combination of any of the above recited values as upper and/or lower limits are intended to be included. Examples of buffers that will control the pH within this range include acetate (e.g. sodium acetate), succinate (such as sodium succinate), gluconate, histidine, citrate and other organic acid buffers.

In certain embodiments, the formulation for use in a method of treating cancer or inhibiting tumor growth in a mammal includes a buffer system which contains citrate and phosphate to maintain the pH in a range of about 4 to about 8. In certain embodiments the pH range may be from about 4.5 to about 6.0, or from about pH 4.8 to about 5.5, or in a pH range of about 5.0 to about 5.2. In certain embodiments, the buffer system includes citric acid monohydrate, sodium citrate, disodium phosphate dihydrate, and/or sodium dihydrogen phosphate dihydrate. In certain embodiments, the buffer system includes about 1.3 mg/mL of citric acid (e.g., 1.305 mg/mL), about 0.3 mg/mL of sodium citrate (e.g., 0.305 mg/mL), about 1.5 mg/mL of disodium phosphate dihydrate (e.g., 1.53 mg/mL), about 0.9 mg/mL of sodium dihydrogen phosphate dihydrate (e.g., 0.86 mg/mL), and about 6.2 mg/mL of sodium chloride (e.g., 6.165 mg/mL). In certain embodiments, the buffer system includes about 1-1.5 mg/mL of citric acid, about 0.25 to about 0.5 mg/mL of sodium citrate, about 1.25 to about 1.75 mg/mL of disodium phosphate dihydrate, about 0.7 to about 1.1 mg/mL of sodium dihydrogen phosphate dihydrate, and 6.0 to 6.4 mg/mL of sodium chloride. In certain embodiments, the pH of the formulation is adjusted with sodium hydroxide.

A polyol, which acts as a tonicifier and may stabilize the antibody, may also be included in the formulation. The polyol is added to the formulation in an amount which may vary with respect to the desired isotonicity of the formulation. In certain embodiments, the aqueous formulation may be isotonic. The amount of polyol added may also alter with respect to the molecular weight of the polyol. For example, a lower amount of a monosaccharide (e.g. mannitol) may be added, compared to a disaccharide (such as trehalose). In certain embodiments, the polyol which may be used in the formulation as a tonicity agent is mannitol. In certain embodiments, the mannitol concentration may be about 5 to about 20 mg/mL. In certain embodiments, the concentration of mannitol may be about 7.5 to about 15 mg/mL. In certain embodiments, the concentration of mannitol may be about 10-about 14 mg/mL. In certain embodiments, the concentration of mannitol may be about 12 mg/mL. In certain embodiments, the polyol sorbitol may be included in the formulation.

A detergent or surfactant may also be added to the formulation. Exemplary detergents include nonionic detergents such as polysorbates (e.g. polysorbates 20, 80 etc.) or poloxamers (e.g., poloxamer 188). The amount of detergent added is such that it reduces aggregation of the formulated antibody and/or minimizes the formation of particulates in the formulation and/or reduces adsorption. In certain embodiments, the formulation may include a surfactant which is a polysorbate. In certain embodiments, the formulation may contain the detergent polysorbate 80 or Tween 80. Tween 80 is a term used to describe polyoxyethylene (20) sorbitanmonooleate (see Fiedler, Lexikon der Hilfsstoffe, Editio Cantor Verlag Aulendorf, 4th edi., 1996). In certain embodiments, the formulation may contain between about 0.1 mg/mL and about 10 mg/mL of polysorbate 80, or between about 0.5 mg/mL and about 5 mg/mL. In certain embodiments, about 0.1% polysorbate 80 may be added in the formulation.

In addition to aggregation, deamidation is a common product variant of peptides and proteins that may occur during fermentation, harvest/cell clarification, purification, drug substance/drug product storage and during sample analysis. Deamidation is the loss of NH₃ from a protein forming a succinimide intermediate that can undergo hydrolysis. The succinimide intermediate results in a 17 u mass decrease of the parent peptide. The subsequent hydrolysis results in an 18 u mass increase. Isolation of the succinimide intermediate is difficult due to instability under aqueous conditions. As such, deamidation is typically detectable as 1 u mass increase. Deamidation of an asparagine results in either aspartic or isoaspartic acid. The parameters affecting the rate of deamidation include pH, temperature, solvent dielectric constant, ionic strength, primary sequence, local polypeptide conformation and tertiary structure. The amino acid residues adjacent to Asn in the peptide chain affect deamidation rates. Gly and Scr following an Asn in protein sequences results in a higher susceptibility to deamidation.

In certain embodiments, the liquid formulation for use in a method of treating cancer or inhibiting tumor growth in a mammal of the present disclosure may be preserved under conditions of pH and humidity to prevent deamidation of the protein product.

The aqueous carrier of interest herein is one which is pharmaceutically acceptable (safe and non-toxic for administration to a human) and is useful for the preparation of a liquid formulation. Illustrative carriers include sterile water for injection (SWFI), bacteriostatic water for injection (BWFI), a pH buffered solution (e.g. phosphate-buffered saline), sterile saline solution, Ringer's solution or dextrose solution.

A preservative may be optionally added to the formulations herein to reduce bacterial action. The addition of a preservative may, for example, facilitate the production of a multi-use (multiple-dose) formulation.

Intravenous (IV) formulations may be the preferred administration route in particular instances, such as when a patient is in the hospital after transplantation receiving all drugs via the IV route. In certain embodiments, the liquid formulation is diluted with 0.9% Sodium Chloride solution before administration. In certain embodiments, the diluted drug product for injection is isotonic and suitable for administration by intravenous infusion.

In certain embodiments, a salt or buffer components may be added in an amount of 10 mM-200 mM. The salts and/or buffers are pharmaceutically acceptable and are derived from various known acids (inorganic and organic) with “base forming” metals or amines. In certain embodiments, the buffer may be phosphate buffer. In certain embodiments, the buffer may be glycinate, carbonate, citrate buffers, in which case, sodium, potassium or ammonium ions can serve as counterion.

In one embodiment, the liquid formulation contains 10 mg/mL M6903, 8% (w/v) Trehalose, 10 mM L-Histidine and 0.05% Polysorbate 20, pH 5.5. Prior to administration of M6903 by intravenous infusion, the solution is diluted in sterile 0.9% sodium chloride.

Lyophilized Formulation

The lyophilized formulation for use in a method of treating cancer or inhibiting tumor growth in a mammal of the present disclosure includes the anti-TIM-3 antibody molecule and a lyoprotectant. The lyoprotectant may be sugar, e.g., disaccharides. In certain embodiments, the lycoprotectant may be sucrose or maltose. The lyophilized formulation may also include one or more of a buffering agent, a surfactant, a bulking agent, and/or a preservative.

The amount of sucrose or maltose useful for stabilization of the lyophilized drug product may be in a weight ratio of at least 1:2 protein to sucrose or maltose. In certain embodiments, the protein to sucrose or maltose weight ratio may be of from 1:2 to 1:5.

In certain embodiments, the pH of the formulation, prior to lyophilization, may be set by addition of a pharmaceutically acceptable acid and/or base. In certain embodiments the pharmaceutically acceptable acid may be hydrochloric acid. In certain embodiments, the pharmaceutically acceptable base may be sodium hydroxide.

Before lyophilization, the pH of the solution containing the protein of the present disclosure may be adjusted between about 6 to about 8. In certain embodiments, the pH range for the lyophilized drug product may be from about 7 to about 8.

In certain embodiments, a salt or buffer components may be added in an amount of about 10 mM-about 200 mM. The salts and/or buffers are pharmaceutically acceptable and are derived from various known acids (inorganic and organic) with “base forming” metals or amines. In certain embodiments, the buffer may be phosphate buffer. In certain embodiments, the buffer may be glycinate, carbonate, citrate buffers, in which case, sodium, potassium or ammonium ions can serve as counterion.

In certain embodiments, a “bulking agent” may be added. A “bulking agent” is a compound which adds mass to a lyophilized mixture and contributes to the physical structure of the lyophilized cake (e.g., facilitates the production of an essentially uniform lyophilized cake which maintains an open pore structure). Illustrative bulking agents include mannitol, glycine, polyethylene glycol and sorbitol. The lyophilized formulations of the present invention may contain such bulking agents.

A preservative may be optionally added to the formulations herein to reduce bacterial action. The addition of a preservative may, for example, facilitate the production of a multi-use (multiple-dose) formulation.

In certain embodiments, the lyophilized drug product for use in a method of treating cancer or inhibiting tumor growth in a mammal may be constituted with an aqueous carrier. The aqueous carrier of interest herein is one which is pharmaceutically acceptable (e.g., safe and non-toxic for administration to a human) and is useful for the preparation of a liquid formulation, after lyophilization. Illustrative diluents include sterile water for injection (SWFI), bacteriostatic water for injection (BWFI), a pH buffered solution (e.g. phosphate-buffered saline), sterile saline solution, Ringer's solution or dextrose solution.

In certain embodiments, the lyophilized drug product of the current disclosure is reconstituted with either Sterile Water for Injection, USP (SWFI) or 0.9% Sodium Chloride Injection, USP. During reconstitution, the lyophilized powder dissolves into a solution.

In certain embodiments, the lyophilized protein product of the instant disclosure is constituted to about 4.5 mL water for injection and diluted with 0.9% saline solution (sodium chloride solution).

Practice of the invention will be more fully understood from the foregoing examples, which are presented herein for illustrative purposes only, and should not be construed as limiting the invention in any way.

### EXAMPLES

### Example 1—Generation and Characterization of Anti-TIM-3 Antibodies

1.1 Generation of Transient and Stable Cell Lines Expressing Human and Cynomolgus Monkey TIM-3

Methods standard in the art were used to generate cell lines expressing membrane-anchored TIM-3. Briefly, to generate huTIM-3-Expi293F cells (also referred to as 293F-hTIM-3) or cynoTIM-3-Expi293F cells (also referred to as 293F-cynoTIM-3)the cDNA encoding the extracellular domains (ECD) of human TIM-3 (based on NCBI reference NP_116171 (SEQ ID NO: 41)) or cyno TIM-3 (based on NCBI reference XP_005558438 (SEQ ID NO: 42)) respectively were obtained by de novo gene synthesis, introduced into an expression vector, and the respective DNAs were transfected into Expi293F cells using Expifectamine (ThermoFischer). Empty vector was used as control. After 3 days, human TIM-3 or cyno TIM-3 cell surface expression were assessed by FACS (for human TIM-3: anti-human TIM-3 antibody (R&D Systems cat # FAB2365P) and a rat IgG2 control-PE (R&D Systems cat # IC006P); for cyno TIM-3: anti-human TIM-3 antibody (Biolegend, cat #345010) and a mouse IgG1 control (Sigma, cat # M9269)) and cell banks were generated. Thawed cells showed no decrease in human or cyno TIM-3 surface expression (data not shown).

To generate to huTIM-3-CHO-S cells (also referred to as CHO-S-hTIM3) CHO-S cells were transfected using a Nucleofector II Device (Amaxa Biosystems) with the same vector described above and selected with hygromycin B. Minipools were screened for cell surface expression of human TIM-3 using FACS. Single cells from the best minipools were sorted by FACS, expanded, and the clone with the highest expression of human TIM-3 was selected (data not shown).

To generate cell lines expressing recombinant soluble TIM-3, the cDNA encoding for the ECD of cynomolgus monkey (“cyno”) TIM-3 based on NCBI reference XP_005558438 corresponding to SEQ ID NO: 42 was obtained by de novo gene synthesis and fused to the DNA encoding either murine Fc or a 6-His tag, and expression vectors containing the cyno TIM-3-muFc and cyno TTM-3-His6 construct were prepared using standard recombinant DNA techniques. DNA was transfected into HEK293 cells using PEI for transient expression.

1.2 Protein Reagents

The cyno TIM-3 ECD proteins was purified from cell supernatant by either protein A affinity (the cyno TIM-3-muFc) or Nickel chelating affinity column and elution with imidazole (cyno TIM-3-His6). QC analysis was performed on the purified proteins: SDS PAGE under reducing and non-reducing conditions, SEC for determination of purity and apparent MW, UV spectroscopy for concentration determination, and Limulus Amcbocytc Lysate assay for measurement of endotoxin contamination.

The human TIM-3 ECD with 6-His tag (hu TIM-3-His6) was purchased from Novoprotein (Cat # C356, SEQ ID NO: 43), human TIM-3 ECD fused to human Fc-6His hu TIM-3-FcHis6) was purchased from Novoprotein (Cat # CD71, SEQ ID NO: 44), human TIM-3 ECD fused to human IgG1 Fc domain (huTIM-3-Fc) was purchased from R&D Systems (#2365-TM), the marmoset TIM-3 ECD with 6-His tag (marmoset TIM-3-His6) was purchased from Novoprotein (Cat # CM64, SEQ ID NO: 45), the mouse TIM-3 ECD fused to human Fc (mouse TIM-3-Fc) was purchased from R&D Systems (#1523-TM) based on NCBI reference NP_599011 (SEQ ID NO: 46), human TIM-1 ECD with 6-His tag (huTIM-1-His6, #1750-TM) and human TIM-4 ECD with 6-His tag (huTIM-4-His6; #2929-TM) were purchased from R&D Systems.

1.3 Animals

Anti-TIM-3 human monoclonal antibodies were generated using transgenic rats (OmniRats™ licensed from Open Monoclonal Technologies, Inc./Ligand Pharmaceutical Inc.) that express human antibody genes: human light chain (VLCL or VKCK) and human VH while expressing the rat constant regions of the heavy chain (Geurts et al. (2009) SCIENCE 325(5939):433, Menoret et al. 2010, Ma et al. 2013, Osborn et al. 2013).

1.4 Generation of Anti-TIM-3 Antibodies from B Cell Cloning

To generate fully human monoclonal antibodies to TIM-3, OmniRats™ were immunized with hu TIM-3-His6 (Novoprotein Cat # C356). General immunization schemes were used for either standard or Repetitive IMmunization at Multiple Sites (also known as RIMMS). Eight to twelve weeks old rats were immunized biweekly four times with hu TIM-3-His6 and serum immune response was monitored by FACS on huTIM-3-Expi293F cells. Briefly, cells were incubated for 20 min at 4° C. with dilutions of the sera, centrifuged, washed and incubated with a mixture of FITC-conjugated goat anti-rat IgG1 (Bethyl # A110-106F) and FITC-conjugated goat anti-rat IgG2b (Bethyl # A110-111F) for 20 min at 4° C. Cells were then centrifuged, resuspended in 7-AAD dye (BD Biosciences) to label dying or dead cells, and analysed using a Guava reader (MilliporeSigma).

Single B cell sorting was performed from lymphocytes collected from rats with high serum immune response. In short, cells were incubated with anti-rat CD32 (clone D34-485, BD Biosciences) for 5 minutes followed by hu TIM-3-His6 for 1 hour at 4° C. Cells were then washed and incubated with a mixture of FITC-conjugated mouse anti-rat IgM (clone MARM-4, ThermoFisher), PE-Cy7-conjugated mouse anti-rat CD45R (clone HIS 24, eBioscience), and APC-conjugated mouse anti-His (clone AD1.1.10R, R&D) antibodies for 30 minutes at 4° C. Single TIM-3 positive B cells were sorted into each well of a 96 well plate containing 4 μl lysis buffer (0.1M DTT, 40 U/ml Rnase Inhibitor, Invitrogen, Cat #10777-019) on BD FACS Aria III flow cytometer. Plates were sealed with Microseal ‘F’ Film (BioRad) and immediately frozen on dry ice before storage at −80° C.

Ig variable (V) region gene-cloning from single sorted B cell was performed with a protocol modified from published reference (Tiller et al., 2008, J Imm Methods 329). In brief, total RNA from single sorted B cells was reverse transcribed in a final volume of 14 μl/well in the original 96-well sorting plate with nuclease-free water (Invitrogen, Cat # AM9935) using final amounts/concentrations of 150 ng random hexamer primer (pd(N)6, Applied Biosystems, P/N N808-0127) and 50U Superscript III reverse transcriptase (Invitrogen, Cat #18080-044) following manufacture protocol. Primers were modified based on previous publications (Max et al., 1981; Weiss and Wu, 1987; Sanchez et al., 1990; Solin and Kaartinen, 1992; Kantor et al., 1997a; Thiebe et al., 1999; Wang et al., 2000; Brekke and Garrard, 2004; Ye, 2004; Ehlers et al., 2006; Johnston et al., 2006; Casellas et al., 2007) and/or designed by examining published Ig gene segment nucleotide sequences from IMGT®, the international ImMuno-GeneTics information system® (see website www.imgt.org; (Lefrane et al., 2009) and NCBI (see website www.ncbi.nlm.nih.gov/igblast/) databases. Human Igh, Igk and Igl V gene transcripts were amplified independently by two rounds of nested (Igh, Igk and Igl) PCR starting from 3.5 μl of cDNA as template. All PCR reactions were performed in 96-well plates in a total volume of 40 μl per well using AccuPrime Taq DNA Polymerase High Fidelity kit, (Invitrogen, Cat #. 12346-094) following manufacture protocol. The first round of PCR was performed at 95° C. for 2 min followed by 40 cycles of 94° C. for 30 s, 50° C. for 30 s, 72° C. for 40 s, and final incubation at 72° C. for 5 min.

Nested second round PCR was performed with 5 μl of unpurified first round PCR product at 95° C. for 2 min followed by 5 cycles of 94° C. for 30 s, 42° C. for 30 s, 72° C. for 45 s, and then 50 cycles of 94° C. for 30 s, 55° C. for 30 s, 72° C. for 45 s, and final incubation at 72° C. for 5 min. PCR products were clones into IgG expression vectors for Ig expression and functional screening.

Total of 352 TIM-3 positive B cells were sorted and Ig Vs cloned into IgG expression vectors for screening. 74 unique clones were isolated with both VH-and VL gene in pair. 74 clones were confirmed by ELISA and flow cytometry assays as human TIM-3 binders, and 10 of these clones were confirmed as cyno-cross reactive cell binders.

1.5 Antibody Expression and Purification

Antibody heavy and light chains were subcloned separately into the pTT5 vector and were transiently co-expressed in Expi293F cells after transfection using the ExpiFectamine transfection reagent. Cells were incubated for 7 days with shaking at 37° C. in a 5% CO2 humidified incubator. Conditioned medium was harvested and centrifuged to remove cell debris. The antibodies were purified from culture supernatants by Protein A affinity chromatography using standard methods. The quality of the purified proteins was assessed by SDS PAGE under reducing and non-reducing conditions, SEC-HPLC was used to determine purity and apparent MW, and the concentration was determined by UV spectroscopy.

1.6 Binding to Human, Cyno, Mouse TIM-3, Human TIM-1 and Human TIM-4 by ELISA

To further confirm the binding to TIM-3, cross-species reactivity and selectivity (against the family members TIM-1 and TIM-4) of the antibodies, the 74 purified antibody clones were tested by ELISA. Briefly 384-well plates were coated overnight with huTIM-3-His6, cynoTIM-3-muFc or cynoTIM-3-His6, mouse TIM-3-Fc, huTIM-1-His6 and huTIM-4-His6. After blocking with 3% Bovine Serum albumin, 1 or 0.1 μg/ml of anti-TIM-3 antibodies were incubated for 1 h at room temperature. Following washing steps, the bound antibodies were incubated for 1 h at room temperature with a peroxidase affiniPure F(ab′)2 Fragment goat anti-human F(ab′)₂ fragment specific (Jackson ImmunoResearch Laboratories #109-036-097) and detection was performed using the TMB HRP Substrate solution (BioFx Lab # TMBW-1000-01). All purified antibody clones were confirmed to bind to human TIM-3, 10 bound strongly to cyno TIM-3 and no antibody clones bound to mouse TIM-3, human TIM-1, or human TIM-4 (data not shown).

1.7 Cell-Based Binding Assays for Anti-TIM-3 Antibodies

Binding of the 74 purified anti-TIM-3 antibody clones to cell lines was assessed by flow cytometry. Briefly, approximately 1×10⁵ CHO-S-hTIM3 cells, parental CHO-S cells, 293F-cynoTIM-3 cells and parental Expi293F were resuspended in flow cytometry buffer (DPBS with 1% FB S) containing anti-TIM-3 antibodies (at 10 μg/ml and 1 ug/ml) and incubated for 30 min on ice. Cells were washed and resuspended in flow cytometry buffer containing FITC-conjugated goat anti-human IgG Fc antibody (Jackson ImmunoResearch Laboratories #109-096-098) for 30 min on ice. Cells were then centrifuged and resuspended in flow cytometry buffer containing 7-AAD and 1% neutral buffered formalin Analysis was done on a Guava EasyCyte instrument (MilliporeSigma). Mean Fluorescence Intensity (MFI) of gated single live cells was calculated for each antibody concentration. A total of 62 antibody clones bound specifically to CHO-S-hTIM3 cells both at 10 and 1 ug/ml, and 12 antibody clones bound specifically to 293F-cynoTIM-3 cells at both 10 and 1 ug/ml (data not shown). Based on ELISA and cell-based binding assay data, 10 anti-TIM-3 antibody clones were selected for further testing and analysis.

1.8 EC50 Measurement by Flow Cytometry and ELISA of Selected Anti-TIM3 Antibody Clones

The selected anti-TIM-3 antibody clones were further tested by flow cytometry and ELISA to calculate their EC50 values in order to rank them. For flow cytometry, the antibodies were tested using protocol described in Example 1.7 using a serial dilution of antibodies starting at 600 nM against cells expressing human and cyno TIM-3. The Mean Fluorescence Intensity (MFI) was plotted against antibody concentration and GraphPad Prism was used to calculate the EC50. Results are summarized in TABLE 1.

For the ELISA, antibodies were tested for binding to huTIM-3-His6, cynoTIM-3-His6 and cynoTIM-3-muFc using serial dilutions starting at 20 nM and following the protocol described in Example 1.6. The Optical Density was plotted against antibody concentration and GraphPad Prism was used to calculate the EC50. Results are summarized in TABLE 1.

Data in TABLE 1 for selected clones show cell-binding to CHO-S-hTIM3 cells with an EC50 of approximately 1 nM and to 293F-cynoTIM-3 cells with EC50 from 2 nM to 183 nM, depending on the antibody clone. These antibody clones bound by ELISA to recombinant human TIM-3 with EC50 of approximately 0.01 nM and to recombinant cyno TIM-3 with EC50 from 0.01 to 1 nM.

1.9 Determination by Surface Plasmon Resonance (SPR) of Kinetic Constants of Selected Anti-TIM-3 Antibodies

Binding affinities of selected anti-TIM-3 antibodies to human TIM-3 and cyno TIM-3 were measured by Surface Plasmon Resonance (SPR) using a GE Healthcare Biacore 4000 instrument as follows. Goat anti-human Fc antibody (Jackson Immunoresearch Laboratories #109-005-098) was first immobilized on BIAcore carboxymethylated dextran CM5 chip using direct coupling to free amino groups following the procedure described by the manufacturer. Antibodies were then captured on the CM5 biosensor chip to achieve approximately 200 response units (RU). Binding measurements were performed using the running HBS-EP+ buffer. A 2-fold dilution series starting at 100 nM of His-tagged TIM-3 proteins, huTIM-3-His6 and cynoTIM-3-His6 were injected at a flow rate of 30 μl/min at 25° C. Association rates (kon, M-1s-1) and dissociation rates (koff, s-1) were calculated using a simple 1:1 Langmuir binding model (Biacore 4000 Evaluation Software). The equilibrium dissociation constant (KD, M) was calculated as the ratio of koff/kon. Affinity of selected clones for binding to huTIM-3-His6 ranged from 3.5 to 9.2 nM (TABLE 1). Some selected clones had affinity for binding to cyno TIM-3-His6 ranging from 12 to 99 nM (TABLE 1). Within the concentration range of cyno TIM-3-His6 tested (100 nM and lower) some clones did not have detectable or determinable specific binding kinetic curves (NB in TABLE 1), indicating weak or no binding to cyno TIM-3-His6 under these conditions. Note that all selected clones shown in TABLE 1 had some binding to cyno TIM-3-His6 under the ELISA or cell-binding flow cytometry conditions.

1.10 ADCC with CHO-S-hTIM3 Target Cells

Selected clones were also tested for ADCC activity using stably transfected CHO-S-hTIM3 target cells and donor effector cells with the allotype V/F using the Chromium release assay.

Briefly, CHO-S-hTIM3 cells were first labeled with ⁵¹Cr for 45 min, then incubated for 15 min at 37° C. with 5-fold serial dilutions of anti-TIM-3 antibodies at the starting concentration of 33 nM. Effector cells were added at the ratio of 1:100 and incubated for 4 hours at 37° C. Cells were transferred to Lumaplate 96 well DryPlates overnight and radioactivity was measured using a gamma counter. The percent lysis was calculated as the ratio of ((Count-Spont)/(100% Lysis-Spont))×100 where Spont is the radioactivity counted with the CHO-S-hTIM3 cells alone (in the absence of antibody and effector cells) and 100% lysis was calculated by lysing the CHO-S-hTIM3 cells with detergent. The assay was done with two donors with the allotype V/F. All selected antibody clones induced ADCC of the CHO-S-hTIM3 target cells with EC50 ranging from about 0.1 to 0.3 nM (TABLE 1).

TABLE 1
Examples of Cell-binding EC50, ELISA EC50, Affinity and
ADCC activity of selected anti-h TIM3 antibody clones

| | | | | | | | | ADCC | |
|---|---|---|---|---|---|---|---|---|---|
| | Cell-Binding | | | | | | | (hTIM3- | |
| | (flow cytometry) | | ELISA | | | Affinity (SPR) | | CHO-S | |
| | | 293F- | | cyno | cyno | | cyno | target cell) | |
| | CHO-S- | cynoT | hTIM3- | TIM3- | TIM3- | | TIM3- | Donor | Donor |
| | hTIM3 | IM3 | His | His | mFc | huTIM3-His | His | 1 | 2 |
| | EC50 | EC50 | EC50 | EC50 | EC50 | KD | KD | EC50 | EC50 |
| Clone ID | (nM) | (nM) | (nM) | (nM) | (nM) | (nM) | (nM) | (nM) | (nM) |
| 3901A12 | 0.7 | 6.1 | 0.01 | 0.02 | 0.01 | 9.2 | 99 | 0.16 | 0.12 |
| 3903B11 | 0.4 | 16.6 | 0.01 | 0.16 | 0.06 | 8.8 | NB | 0.10 | 0.08 |
| 3903A04 | 1.0 | 156.1 | 0.01 | 0.74 | 0.08 | 3.5 | NB | 0.29 | 0.18 |
| 3905A01-1 | 1.1 | 183.7 | 0.02 | 1.01 | 0.13 | 3.9 | NB | 0.26 | 0.25 |
| 3903E11 | 0.9 | 2.0 | 0.01 | 0.01 | 0.01 | 3.8 | 12 | 0.24 | 0.11 |

NB = No Binding determinable in the concentration range of cynoTIM3 analyte tested by SPR

### Example 2—Optimization of Anti-Tim-3 Antibody 3903E11

2.1 Heavy and Light Chain Variable Region Variants

The amino acid sequences of the variable regions of 3903E11 heavy chain (3903E11 (VH1.0); SEQ ID NO: 34) and of the variable regions of 3903E11 light chain (3903E11 (VL1.0; SEQ ID NO: 33) chains were separately modified, by altering both framework region and CDR sequences in the heavy and light chain variable regions. The purpose of these sequence alterations was either to mutate framework amino acid residues to the most homologous human germline residue found at that position, to improve manufacturability of the molecule by preventing Asp isomerization, Asn deamidation and Met oxidation, or to deplete the antibody of in silico identified human T-cell epitopes, thereby reducing or abolishing immunogenicity in humans.

Three heavy chain variable region variants 3903E11 (VH1.1) (SEQ ID NO: 53), 3903E11 (VH1.2) (SEQ ID NO: 24), and 3903E11 (VH1.3) (SEQ ID NO: 55) were constructed on a human IgG1 heavy chain isotype backbone, yielding heavy chain variants 3903E11 (VH1.1)-g1 (SEQ ID NO: 16), 3903E11 (VH1.2)-g1 (SEQ ID NO: 18), and 3903E11 (VH1.3)-g1 (SEQ ID NO: 20), respectively. The following mutations were introduced (according to IMGT numbering scheme; residues that are underlined are located in or bordering one of the CDRs):

3903E11 (VH1.1): M39L

3903E11 (VH1.2): Q6E

3903E11 (VH1.3): Q6E, M39L

Three light chain variable region variants 3903E11 (VL1.1) (SEQ ID NO: 52), 3903E11 (VL1.2) (SEQ ID NO: 54), and 3903E11 (VL1.3) (SEQ ID NO: 23) were constructed on a human lambda chain background, yielding light chain variants 3903E11 (VL1.1)-CL (SEQ ID NO: 15), 3903E11 (VL1.2)-CL (SEQ ID NO: 17), and 3903E11 (VL1.3)-CL (SEQ ID NO: 19), respectively. The following mutations were introduced (according to IMGT numbering scheme):

3903E11 (VL1.1): S1Q,Y2S, E3A

3903E11 (VL1.2): F55Y

3903E11 (VL1.3): S1Q, Y2S, E3A, F55Y

The parental and variant heavy and light chains were combined in all possible pair-wise combinations to generate further fully human anti-TIM-3 antibodies. Optimized candidates were selected based on their binding activity to TIM-3 (by FACS and SPR) and similarity to the most homologous human germline residue at FR positions. All optimized antibodies were functional and retained binding to CHO-S-hTIM3 cells and recombinant human and cyno TIM-3 proteins (see TABLE 2). Relative to the parental 3903E11 heavy chain, combinations with the 3903E11 (VH1.2) IgG1 resulted in an antibody with increased binding to CHO-S-hTIM3 cells and increased innate affinity (KD) to both recombinant huTIM-3-His6 and cyno TIM-3-His6 (see TABLE 2). Moreover, light chain variant 3903E11 (VL1.3) CL was more similar to the most homologous germline sequence than the other light chain variants. Thus, the antibody containing heavy chain variable region variant 3903E11 (VH1.2) and light chain variable region variant 3903E11 (VL1.3) was chosen for further characterization as the lead antibody, 3903E11 (VL1.3,VH1.2) IgG1.

TABLE 2
Characterization of sequence-optimized variants

| | | | | | | | | Biacorc Analysis | |
|---|---|---|---|---|---|---|---|---|---|
| | | | | | | | | (relative to | |
| | | | FACS CHO- | | | | | Human TIM3 | |
| | Expression | | S-hTIM-3 | | | | | K_{D} | |
| | & MPP | | MFI | MFI | MFI | Biacore Binding | | Ratio | |
| | | % | at 10 | at 1 | at 0.1 | HuTIM3 | cynoTIM3 | to | HC-34 |
| Clone ID | Titer | Monomer | μg/ml | μg/ml | μg/ml | K_{D}(nM) | K_{D}(nM) | Parent | Identity |
| 3903E11 (VH1.0, VL1.0) | 718 | 91 | 1494 | 1387 | 137 | 3.5 | 6.9 | 1.00 | M |
| 3903E11 (VH1.0, VL1.1) | 392 | 93 | 1873 | 1455 | 148 | 2.7 | 6.5 | 0.78 | |
| 3903E11 (VH1.0, VL1.2) | 606 | 90 | 1932 | 1344 | 140 | 3.4 | 8.6 | 0.97 | |
| 3903E11 (VH1.0, VL1.3) | 1188 | 92 | 1934 | 1545 | 162 | 2.7 | 6.7 | 0.76 | |
| 3903E11 (VH1.1, VL1.0) | 629 | 93 | 1919 | 1146 | 118 | 10.3 | 24.1 | 2.92 | L |
| 3903E11 (VH1.1, VL1.1) | 635 | 93 | 1947 | 1390 | 154 | 7.6 | 17.9 | 2.15 | |
| 3903E11 (VH1.1, VL1.2) | 540 | 91 | 1972 | 1293 | 122 | 8.6 | 42.4 | 2.45 | |
| 3903E11 (VH1.1, VL1.3) | 589 | 92 | 1968 | 1379 | 119 | 7.4 | 22.3 | 2.10 | |
| 3903E11 (VH1.2, VL1.0) | 485 | 93 | 1953 | 1507 | 156 | 2.2 | 5.5 | 0.62 | M |
| 3903E11 (VH1.2, VL1.1) | 547 | 94 | 1927 | 1399 | 148 | 1.8 | 5.6 | 0.52 | |
| 3903E11 (VH1.2, VL1.2) | 560 | 92 | 1899 | 1297 | 114 | 1.6 | 6.3 | 0.46 | |
| 3903E11 (VH1.2, VL1.3) | 635 | 94 | 1908 | 1307 | 140 | 1.6 | 5.3 | 0.46 | |
| 3903E11 (VH1.3, VL1.0) | 643 | 93 | 1932 | 957 | 105 | 6.4 | 19.3 | 1.81 | L |
| 3903E11 (VH1.3, VL1.1) | 514 | 94 | 1929 | 1050 | 105 | 4.7 | 19.5 | 1.35 | |
| 3903E11 (VH1.3, VL1.2) | 571 | 92 | 1910 | 958 | 87 | 6.0 | 19.9 | 1.70 | |
| 3903E11 (VH1.3, VL1.3) | 634 | 93 | 1923 | 1020 | 82 | 4.5 | 19.7 | 1.29 | |

### Example 3—Antibody Production and Characterization

3.1 Bioproduction, Clarification and Purification

Antibody 3903E11 (VL1.3,VH1.2) was produced from CHO-S cells. Cells were grown in a CHO fed-batch growth media supplemented with glucose at 37° C. The cultures were fed with a mixture of feed components on days 3, 5, 7 and 10 days post inoculation.

Crude conditioned media from the bioreactor runs were clarified using 2.2 m2 Millistak+Pod DOHC (Millipore MD0HC10FS1) and 1.1 m2 Millistak+Pod XOHC (Millipore # MX0HC01FS1) filters, followed by terminal filtration with a Millipore Opticap XL3 0.5/0.2 μm filter (Millipore # KHGES03HH3).

The antibody was then purified using standard methods and formulated in 10 mM Histidine, 8% Trehalose, pH 5.5 with 0.05% Tween 20.

3.2 Binding Affinity of Sequence-Optimized Anti-TIM3 Antibody 3903 (VL1.3,VH1.2)

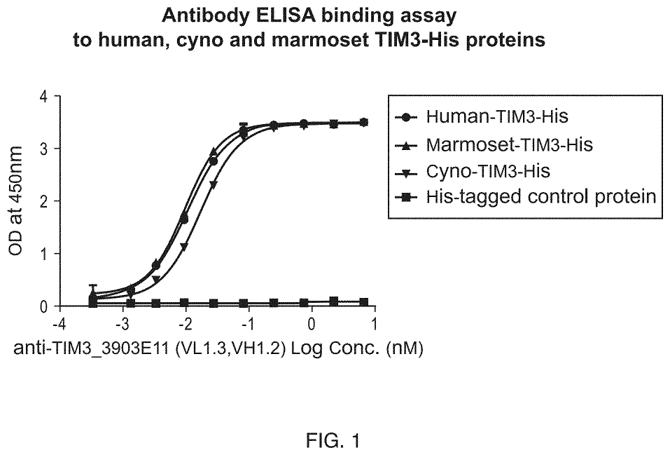

As a non-limiting example, further characterization was performed on anti-TIM3 antibody 3903E11 (VL1.3,VH1.2). Antibody 3903E11 (VL1.3,VH1.2) was tested for binding to huTIM3-His6, cynoTIM3-His6 and marmoset TIM3-His6 proteins following ELISA protocols described in Examples 1.6 and 1.8. 3903E11 (VL1.3,VH1.2) had similar binding by ELISA to human, cyno and marmoset TIM3-His proteins (FIG. 1 and TABLE 3).

3903E11 (VL1.3,VH1.2) antibody was also tested for cell-binding to CHO-hTIM-3 cells by flow cytometry, and binding affinities to human, cyno and marmoset TIM-3 were determined by SPR (protocols described in Examples 1.8 and 1.9). The EC50 of binding to CHO-hTIM-3 cells was 2.6 nM, and equilibrium dissociation constant (KD) affinities determined by SPR were 2.4 nM (human TIM3-His6), 14 nM (cynoTIM3-His6) and 11 nM (marmoset TIM3-His6) (TABLE 3).

TABLE 3
Cell-binding EC50, ELISA EC50, and affinity of
anti-TIM-3 antibody 3903E11 (VL1.3, VH1.2)

| | Cell-Binding | | | | | | |
|---|---|---|---|---|---|---|---|
| | (flow | | | | | | |
| | Cytometry) | ELISA | | | Affinity (SPR) | | |
| | CHO-S- | hTIM3- | CynoTIM3- | marmoset | huTIM3- | CynoTIM3- | marmoset |
| | hTIM-3 | His | His | TIM3-His | His | His | TIM3-His |
| | EC50 | EC50 | EC50 | EC50 | KD | KD | KD |
| Antibody | (nM) | (nM) | (nM) | (nM) | (nM) | (nM) | (nM) |
| 3903E11 | 2.6 | 0.01 | 0.02 | 0.01 | 2.4 | 14 | 11 |
| (VL1.3, VH1.2) | | | | | | | |

3.3. 3903E11 (VL1.3, VL1.2) Efficiently Blocked the Interaction of huTIM3 and Galectin-9.

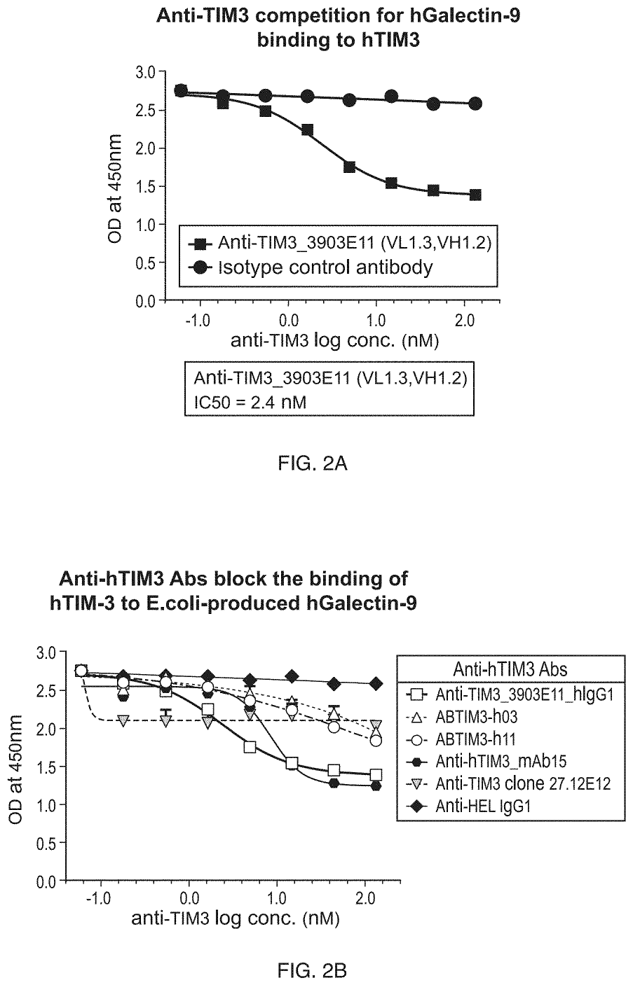

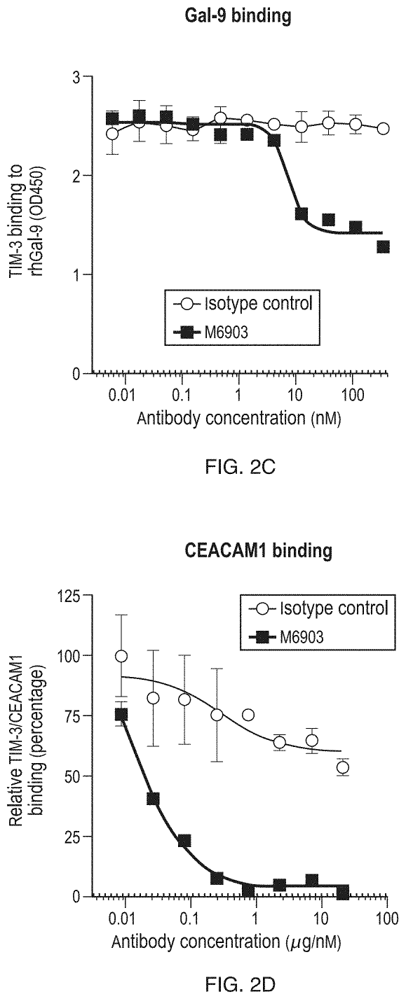

3903E11 (VL1.3,VH1.2) antibody was further tested for inhibition of the binding interaction between TIM-3 and galectin-9. A competition ELISA was used to test the effect of anti-TIM-3 antibodies on galectin-9 binding to TIM-3. Briefly 96-well plates were coated overnight with recombinant human galectin-9 protein (R&D Systems #2045-GA), then washed and blocked with 3% Bovine Serum albumin. HuTIM-3Fc was biotinylated with Sulfo-NHS-LC-Biotin labelling kit (ThermoFisher Scientific #21327). Human-TIM3-biotin (1 μg/ml) was mixed with 3903E11 (VL1.3,VH1.2) antibody or an isotype control antibody in serial dilutions starting at 133 nM, incubated 1 h at room temperature, then the antibody/human-TIM3-biotin mixtures added to plates coated with human galectin-9 and incubated for 2 h at room temperature. Plates were washed, and bound human-TIM-3-biotin detected by incubation with HRP-streptavidin (Jackson ImmunoResearch Laboratories #016-030-084) for 1 h at room temperature and developed using the TMB HRP Substrate solution (BioFx Lab # TMBW-1000-01). Data were plotted and IC50 values calculated using GraphPad Prism. The isotype control antibody had no effect on huTIM-3 binding to human galectin-9, while 3903E11 (VL1.3,VH1.2) antibody produced a dose-dependent blockade of binding between human galectin-9 and huTIM-3, with an IC50 of 2.4 nM (see FIG. 2A).

The experiment was repeated with other anti-TIM-3 antibodies (ABTIM3-h03 and AB TIM3-h11, ABTIM3-mAB 15, and ABTIM3 27.12E1), and the results summarized in FIG. 2B. With the exception of ABTIM3-mAB 15, only 3903E11 (VL1.3,VH1.2) antibody strongly blocked galectin-9 binding to huTIM-3.

3.4 Construction of M6903 Antibody

An effector-negative isotype antibody was designed that, compared to human IgG1 isotype, does not bind to FcγRs, but still has normal binding to FcRn This antibody, designated “anti-TIM-3-3903E11(VL1.3,VH1.2)-IgG2h(FN-AQ,322A)-delK” antibody and is also referred to as M6903, has a lambda light chain and an engineered IgG2 isotype. The heavy chain CH2 region contains two amino acid substitutions designed to abrogate effector function: N297Q (Kabat EU index) to eliminate heavy chain N-glycosylation thereby reducing FcγR binding affinity and ADCC, and K322A (Kabat EU index) to eliminate C1q binding and abolish CDC. An F296A mutation (Kabat EU index) was also introduced to reduce potential immunogenicity. Additionally, the human IgG2 hinge region was replaced with a human IgG1 hinge with a C220S substitution (Kabat EU index) to improve protein stability, and the C-terminal heavy chain lysine was deleted to reduce heterogeneity (“IgG2h(FN-AQ,322A)-delK”). M6903 contains the variable light chain VL1.3 and variable heavy chain VH1.2 (described in Example 2.1) in an IgG2h(FN-AQ,322A)-delK background.

3.5. M6903 Efficiently Blocked the Interaction of huTIM-3 and Galectin-9.

In a competition-based ELISA, M6903, like 3903E11 (VL1.3,VH1.2) antibody (see sec. 3.3 above), inhibited huTIM-3-biotin binding to huGal-9 in a concentration-dependent manner (FIG. 2C), with an IC50 of 7.46±0.052 nM. Calculating the percent blocking revealed that M6903 blocked up to 55% of the TIM-3/Gal-9 binding signal that was obtained in the absence of antibody.

Specifically, recombinant human Gal-9 protein (R&D) was coated at 2 μg/ml onto ELISA assay plates and then blocked with 3% bovine scrum albumin (BSA). Recombinant Human TIM-3-Fc Chimera protein (R&D, 2365-TM) was biotinylated with EZ-Link Sulfo-NHS-LC-Biotinylation kit (Thermo Scientific, 21327). Biotinylated-human TIM-3-Fc (huTIM-3-Fc-biotin) was added to Gal-9 coated assay plates at a final concentration of 0.5 μg/ml huTIM-3-Fc-biotin in assay buffer and detected with Streptavidin Tag Peroxidase-conjugated antibody (Jackson ImmunoResearch) and TMB HRP Substrate (BioFX/SurModics IVD, TMBS-1000-01). To test antibodies for inhibition of Gal-9 binding to TIM-3, huTIM-3-Fc-biotin was mixed with antibodies in an 11-point 1:3 dilution series from 50 μg/ml starting concentration in quadruplicate for each antibody and incubated 50 minutes at room temperature. Following this incubation step, the huTIM-3-Fc-biotin+antibody mixtures were added to the Gal-9 coated ELISA assay plates and incubated for 75 minutes at room temperature, then washed and developed with detection reagents and OD (450 nm) was read.

3.6. M6903 Efficiently Blocked the Interaction of huTIM-3 and CEACAM1

In an ELISA binding assay, binding of TIM-3 with CEACAM1 was measured by incubating soluble recombinant His-tagged CEACAM1 protein with plate-bound recombinant TIM-3-Fc (rhTIM-3-Fc). Pre-incubation of plate-bound rhTIM-3 with M6903 reduced binding of rhTIM-3-Fc to His-tagged CEACAM1 in a dose-dependent manner, with an IC50 of 0.353±0.383 nM (0.053±0.057 μg/mL), whereas pre-incubation with an isotype control had no effect on the binding (FIG. 2D).

Specifically, the interaction of between TIM-3 and CEACAM1 was detected by an ELISA-based assay in the presence of 10 mM CaCL2 (Sigma, C3306-100G). In a flat bottom 96-well MaxiSorp plate, 0.5 μg rhTIM-3-IgG1-Fc in 1xTBS (Thermo Scientific, 28358) with 10 mM CaCL2 (Ca2+) was added to each well and the plate was incubated at 4° C. overnight. The plate was then blocked in 2% BSA TBS (Ca2+) at room temperature with shaking for 1 hour. After the supernatant was removed, M6903 or isotype control (anti-HEL IgG2) in TBS (Ca2+) was added to each well and the plate was incubated at room temperature for 1 hour, and then CEACAM1-poly His (ARCO, CE1-H5220) in TBS (Ca2+) was added to each well and the plate was incubated at room temperature for 1 hour with shaking. The plate was then washed and anti-6XHis-HRP (Biolegend, 652504) in TBS (Ca2+) was added to each well followed by incubation at room temperature for 1 hour with shaking. After washing, TMB was added to each well followed by shaking the plate at room temperature for 20 minutes and adding stop solution to each well. The optical density (450 nm and 570 nm) was read immediately using an EnVision plate reader.

3.7 Measurement of Anti-TIM3 Antibodies Binding of FcγR and FcRn

The FcγR and FcRn binding properties of M6903 were measured and compared to appropriate control antibodies with in vitro FcγR and FcRn binding assays.

Binding of antibodies to FcγR and FcRn was measured on an Octet Red96 (ForteBio) instrument. Specifically, to measure binding to FcγR, antibodies at 200 nM concentration were loaded onto Anti-Human Fab-CHI 2nd generation (FAB2G) sensor tips (ForteBio Part No: 18-5125). For High-affinity FcγR, binding conditions: titrated FcγR in range from 100 nM to 1.56 nM with 7-point 1:2 dilution series; Loading 300 sec, Association 180 sec, Dissociation 300 sec. For Low-affinity FcγR binding conditions: titrated range from 4000 nM to 62.5 nM with 7-point 1:2 dilution series; Loading 300 sec, Association 60 sec., Dissociation 300 sec. Working buffer contained 1% BSA, 0.05% Tween-20, PBS pH7.4. FcγR proteins were purchased from R&D Systems. An effector-negative antibody hu14.18-IgG2h(FN-AQ,K322A) that was previously characterized for no FcγR binding or effector functions was used as a negative control for sensor subtraction. FcγR binding KD values were obtained using Fortebio Data Analysis Software ver. 7.1.0.36.

As expected, M6903 antibody and control antibodies of both IgG1 and the effector-negative isotype “IgG2h(FN-AQ, K322A-delK)” had equivalent binding to FcRn, ranging from 620 nM to 850 nM (TABLE 4). Anti-TIM-3-IgG1 (3903E11) binding affinity KD: 100 nM (CD16a), 630 nM (CD32a) and 1.7 nM (CD64). Anti-GD2 hu14.18-huIgG1 binding affinity KD: 215 nM (CD16a), 670 nM (CD32a) and 1.8 nM (CD64).

M6903 antibody and control antibody of the effector-negative isotype “IgG2h(FN-AQ, K322A-delK)” had no detectable binding to FcγR (TABLE 4).

These results demonstrate that M6903 anti-TIM-3 has no detectable FcγR binding activity, but normal FcRn binding, which are key properties of the effector-negative isotype of anti-TIM3_3903E11 (VL1.3,VH1.2) antibody.

TABLE 4
Measurement of M6903 and control antibodies binding to FcγR and
FcRn

| | | | | FcRn |
|---|---|---|---|---|
| | FCγRI | FCγRIIa | FCγRIIIa | Steady- |
| | (CD64) | (CD32a)_H131 | (CD16a)_V158 | State |
| Protein (MBE) or | Affinity | Steady-State | Steady-State | KD |
| catalog Name | KD (nM) | KD (nM) | KD (nM) | (nM) |
| anti-TIM3- | NB | NB | NB | 850 |
| 3903E11(VL1.3, VII1.2)- | | | | |
| IgG2h(FN- | | | | |
| AQ, 322A)-delK | | | | |
| (M6903) | | | | |
| anti-TIM3- | 1.7 | 630 | 100 | 805 |
| 3903E11(VL1.3, | | | | |
| VH1.2)-IgG1 | | | | |
| hu14.18 IgG2h | NB | NB | NB | 825 |
| (FN-AQ, K322A)- | | | | |
| delK | | | | |
| hu14.18-huIgG1- | 1.8 | 670 | 215 | 620 |
| delK | | | | |

NB = No Binding
Affinity KD determined from binding kinetics as KD = kd/ka
Steady State KD determined from analysis of steady-state binding versus concentration of FcγR or FcRn plots.
KD values are the average of 2 independent determinations.

### Example 4—Epitope Mapping

4.1 Co-Crystallization of TIM-3 with 3903E11 (VL1.3,VL1.2) Fab

A crystal structure of the complex of TIM-3 ECD and the Fab fragment of the 3903E11 (VL1.3,VH1.2) (heavy chain: SEQ ID NO: 47; light chain: SEQ ID NO: 48) was determined. Human TIM-3 (SEQ ID NO: 49 (amino acid); SEQ ID NO: 50 (nucleotide)) was expressed in E. coli inclusion bodies, refolded, and purified by affinity and size exclusion chromatography. The Fab fragment of 3903E11 (VL1.3,VH1.2) was expressed as a His-tagged construct in Expi293F cells and purified by affinity chromatography. The complex of TIM-3 and 3903E11 (VL1.3,VH1.2) Fab fragment was formed and purified by gel filtration chromatography yielding a homogenous protein with a purity greater than 95%.

Crystals of Fab 3903E11 (VL1.3,VH1.2) in complex with human TIM-3 were grown by mixing 0.75 μl protein solution (21.8 mg/mL in 20 mM TrisHCL pH 8.0, 100 mM NaCl) with 0.5 μl reservoirs solution (20% PEG400 (v/v), 0.1 M Tris HCl pH 8.0) at 4° C. using hanging drop vapor diffusion method.

Crystals were flash-frozen and measured at a temperature of 100 K. The X-ray diffraction data was collected at the SWISS LIGHT SOURCE (SLS, Villigen, Switzerland) using cryogenic conditions. The crystals belong to space group C 2 2 21. Data were processed using the programs XDS and XSCALE.

The phase in-formation necessary to determine and analyse the structure was obtained by molecular replacement. The published structures PDB-ID 5F71 and 1NL0 were used as search models for TIM3 and the Fab fragment, respectively. Subsequent model building and refinement was performed according to standard protocols with the software packages CCP4 and COOT. For the calculation of the free R-factor, a measure to cross-validate the correctness of the final model, about 0.9% of measured reflections were excluded from the refinement procedure (see TABLE 5). TLS refinement (using REFMAC5, CCP4) was carried out, which resulted in lower R-factors and higher quality of the electron density map. The ligand parameterisation and generation of the corresponding library files were carried out with CHEMSKETCH and LIBCHECK (CCP4), respectively. The water model was built with the “Find waters”-algorithm of COOT by putting water molecules in peaks of the Fo-Fc map con-toured at 3.0 followed by refinement with REFMAC5 and checking all waters with the validation tool of COOT. The criteria for the list of suspicious waters were: B-factor greater 80 ∈2, 2Fo-Fc map less than 1.2 Å, distance to closest contact less than 2.3 Å or more than 3.5 Å. The suspicious water molecules and those in the ligand binding site (distance to ligand less than 10 Å) were checked manually. The Ramachandran Plot of the final model shows 85.4% of all residues in the most favored region, 13.9% in the additionally al-lowed region, and 0.2% in the generously allowed region. The residues Arg81(A), Arg81(B), Va153(L), Asp153(L), Va153(M), Asp153(M), Val53(N), Val53(O), and Asp153(O) are found in the disallowed region of the Ramachandran plot. They are either confirmed by the electron density map or could not be modelled in another sensible conformation.

TABLE 5
Data collection and processing statistics for TIM3

| X-ray Source | PXI/X06A (SLS¹) |
|---|---|
| Wavelength [Å] | 1.0000 |
| Detector | EIGER X 16M |
| Temperature [K] | 100 |
| Space Group | C 2 2 2₁ |
| | 119.35; 270.12; |
| Cell: a; b; c; [Å] | 197.89 |
| α; β; γ; [°] | 90.0; 90.0; 9.0 |
| Resolution [Å] | 3.06 (3.31-3.06) |
| Unique reflections | 59975 (12146) |
| Multiplicity | 3.8 (3.9) |
| Completeness [%] | 99.0 (96.7) |
| Rsym [%]³ | 7.5 (50.2) |
| Rmeas [%]⁴ | 8.7 (58.3) |
| Mean(I)/sd⁵ | 15.36 (2.95) |

¹SWISS LIGHT SOURCE (SLS, Villigen, Switzerland)
²values in parenthesis refer to the highest resolution
${}^{3} R sym = \frac{{\sum }_{h} {\sum }_{i}^{{n}_{h}} \vert \text{?}}{{\sum }_{h} {\sum }_{i}^{{n}_{h}} {I}_{h , i}} with {\hat{I}}_{h} - \frac{1}{{n}_{h}} {\sum }_{i}^{{n}_{h}} {I}_{h , i}$ where I_{h,i} is the intensity value of the ith measurement of h
${}^{4} R meas = \frac{{\sum }_{h} \sqrt{\frac{{n}_{h}}{{\text{?}}_{h}}} {\sum }_{i}^{{n}_{h}} \vert \text{?}}{{\sum }_{h} {\sum }_{i}^{{n}_{h}} {I}_{h , i}} with {\hat{I}}_{h} = \frac{1}{{n}_{h}} {\sum }_{i}^{{n}_{h}} {I}_{h , i}$ where I_{h,i} is the intensity value of the ith measurement of h
⁵calculated with independent reflections
$\text{?} \text{indicates text missing or illegible when filed}$

Epitope residues are defined as all residues of TIM-3 with a heavy atom within 5 angstroms of a heavy atom of 3903E11 (VL1.3,VH1.2) Fab. Distances were measured from the final crystallographic coordinates using the BioPython package. Only contacts present in 3 of the 4 complexes of the asymmetric unit are reported (TABLE 6). TABLE 6 tabulates interactions between TIM-3 and 3903E11 (VL1.3,VH1.2). TIM-3 residues are numbered as in Uniprot Code Q8TDQ0-1 (SEQ ID NO: 51). The antibody residues are numbered with reference to SEQ ID NO:47 (heavy chain, “H”) and SEQ ID NO:48 (light chain, “L”). Residues listed here have at least one heavy atom within 5 angstroms of a heavy atom across the interface.

TABLE 6
Interactions between huTIM-3 and mAb 3903E11
(VL1.3, VH1.2)

| huTIM-3 | | 3903E11 (VL1.3, VH1.2) | | |
|---|---|---|---|---|
| Amino | | Amino | | |
| Acid | Number | Acid | Number | Chain |
| PRO | 50 | SER | 54 | H |
| LYS | 55 | TYR | 34 | L |
| GLY | 56 | TYR | 34 | L |
| ALA | 57 | TYR | 32 | L |
| | | TYR | 34 | L |
| CYS | 58 | TYR | 34 | L |
| | | ALA | 94 | L |
| PRO | 59 | TRP | 101 | H |
| | | TYR | 34 | L |
| | | TYR | 93 | L |
| | | ALA | 94 | L |
| VAL | 60 | TRP | 47 | H |
| | | TYR | 59 | H |
| | | TRP | 101 | H |
| | | GLY | 102 | H |
| | | TYR | 93 | L |
| | | ALA | 94 | L |
| | | ASP | 95 | L |
| | | SER | 96 | L |
| | | VAL | 97 | L |
| PHE | 61 | ALA | 33 | H |
| | | SER | 35 | H |
| | | TRP | 47 | H |
| | | ALA | 50 | H |
| | | TYR | 59 | H |
| | | ALA | 99 | H |
| | | ASN | 100 | H |
| | | TRP | 101 | H |
| | | GLY | 102 | H |
| | | PHE | 104 | H |
| | | VAL | 97 | L |
| GLU | 62 | ALA | 33 | H |
| | | SER | 52 | H |
| | | VAL | 53 | H |
| | | TYR | 59 | H |
| | | ASN | 100 | H |
| | | TRP | 101 | H |
| CYS | 63 | TRP | 101 | H |
| GLY | 64 | TRP | 101 | H |
| | | TYR | 34 | L |
| ASN | 65 | TRP | 101 | H |
| | | ASP | 52 | L |
| | | LYS | 55 | L |
| VAL | 66 | TRP | 101 | H |
| ARG | 69 | SER | 31 | H |
| | | VAL | 53 | H |
| GLU | 72 | SER | 54 | H |
| ARG | 111 | TYR | 59 | H |
| GLN | 113 | SER | 52 | H |
| | | VAL | 53 | H |
| | | SER | 54 | H |
| | | SER | 57 | H |
| | | TYR | 59 | H |
| ILE | 114 | GLY | 56 | H |
| | | SER | 57 | H |
| PRO | 115 | GLY | 56 | H |
| | | SER | 57 | I |
| GLY | 116 | GLY | 56 | H |
| | | SER | 57 | H |
| | | THR | 58 | H |
| ILE | 117 | THR | 58 | H |
| | | TYR | 60 | H |
| | | LYS | 65 | H |
| MET | 118 | SER | 57 | H |
| | | THR | 58 | H |
| | | TYR | 59 | H |
| | | TYR | 60 | H |
| | | LYS | 65 | H |
| ASN | 119 | SER | 57 | H |
| ASP | 120 | SER | 57 | H |
| | | TYR | 59 | H |
| LYS | 122 | ASP | 95 | L |

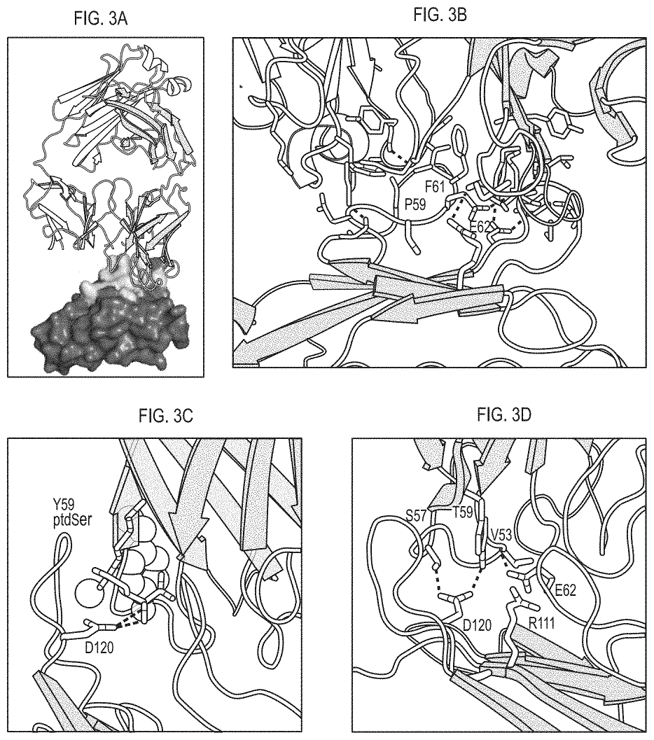

The crystal structure of human TIM-3 in complex with M6903 is shown in FIG. 3A-D. FIG. 3A shows an overview of the Fab portion of M6903 (upper structure) bound to TIM-3 shown as a surface representation. Extensive contacts made on TIM-3 (bottom structure) are shown as the lighter portion of TIM-3. The majority of the contact occurs with the heavy chain and the third complementarity determining region of the light chain (CDR-L3) of M6903. FIG. 3B shows the epitope hotspot residues of TIM-3 (e.g., P59 and F61 and E62). The residues form extensive hydrophobic and electrostatic interactions to M6903. FIG. 3C shows the polar head group of ptdSer (light-colored sticks) and the coordinating calcium ion (sphere) have been modeled into the structure of M6903-bound TIM-3 by superposition with the structure of murine TIM-3 (DeKruyff et al. (2010), supra). The binding site of ptdSer coincides with the placement of Y59 (group of spheres) of the heavy chain from M6903. Hydrogen bonds from D120 on TIM-3 to ptdSer or M6903, respectively, are shown as dotted lines. FIG. 3D shows the polar interactions of M6903 with the CEACAM-1 binding residues of TIM-3 are shown with dashed lines.

4.2 Mutagenesis

To identify residues of the epitope which contribute energetically to binding selected residues in human TIM-3 were mutated either to alanine (large to small) or to glycine if the selected residue was alanine or to switch the charge of the side-chain In total 11 human TIM-3 point mutants were designed, expressed and purified in HEK cells, and tested for binding to M6903 using surface plasmon resonance as described in Example 1.9. The affinity of the antibody for wild-type and each mutant was determined. Results are summarized in TABLE 7.

Mutants were compared to wild-type TIM-3 (hu TIM-3). The temperature midpoint of fluorescently monitored thermal denaturation is given for the wild-type and mutant proteins. The percent monomer as determine by analytical SEC is given. For KD and T1/2, the mean and standard deviation is given where n>1. It was important to confirm that the lack of binding for a particular point mutant was indeed due to loss of residue interaction and not to global unfolding of the antigen. The structural integrity of the mutated proteins was confirmed using a fluorescence monitored thermal unfolding (FMTU) assay in which the protein is incubated with a dye that is quenched in aqueous solution but fluoresces when bound by exposed hydrophobic residues. As the temperature increases, thermal denaturation of the protein exposes the hydrophobic core residues and this can be monitored by an increase in fluorescence of the dye. A melting curve is fit to the data with the Boltzmann equation outlined in Equation 1, adapted from (Bullock et al. 1997) to determine the temperature at the inflection point of the curve (T1/2). The calculated T1/2 are reported in TABLE 7.

$$F = {F}_{min} + \frac{{F}_{max} - {F}_{min}}{1 + {e}^{\frac{{T}_{m} - x}{dx}}} \qquad Equation 1$$

TABLE 7
Summary of TIM-3 Variant Binding to Antibodies

| | Binding Affinity KD (nM) | | | | ΔΔGmut (kcal/mol) | | | | Stability | |
|---|---|---|---|---|---|---|---|---|---|---|
| | | 27.12 | | | | 27.12 | | | % | T_{1/2} |
| Ligand | M6903 | E12 | h03 | mab15 | M6903 | E12 | h03 | mab15 | Monomer | (° C.) |
| TIM3 | 6.2 ± 1.5 | 51.6 ± 10.8 | 0.3 ± 0.5 | 0.7 ± 0.2 | NA | NA | NA | NA | 94 | 52 |
| P59A | NB | 12.3 ± 1.9 | 0.9 ± 0.03 | 0.7 ± 0.2 | >1.6 | −0.2 | 0.6 | −0.03 | 83 | 48 |
| V60A | 3.7 ± 0.1 | 23.4 ± 1.0 | 0.4 ± 0.04 | 0.7 ± 0.4 | −0.3 | −0.2 | 0.2 | −0.05 | 94 | 51 |
| F61A | NB | 28.6 ± 1.2 | 0.6 ± 0.09 | 0.9 ± 0.3 | >1.6 | −0.1 | 0.3 | −0.1 | 100 | nd |
| E62A | 106.1 ± 32 | 28.3 ± 0.2 | 0.4 ± 0.05 | 0.5 ± 0.1 | >1.6 | −0.3 | 0.1 | −0.3 | 97 | 51 |
| R111A | | | | | | | | | 23 | nd |
| R111E | | | | | | | | | 83 | nd |
| I114A | 29.3 ± 0.7 | 26.7 ± 2.6 | 0.7 ± 0.01 | 0.6 ± 0.1 | 0.9* | −0.1 | 0.5 | −0.1 | 95 | nd |
| M118A | 9.7 ± 0.6 | 49.7 ± 4.5 | 0.7 ± 0.09 | 1.1 ± 0.4 | 0.3 | −0.04 | 0.5 | 0.2 | 99 | nd |
| N119A | 17.2 ± 0.7 | 29.1 ± 0.3 | 0.7 ± 0.08 | 1.2 ± 0.4 | 0.6 | −0.3 | 0.4 | 0.3 | 79 | nd |
| K122A | 46.6 ± 0.2 | 22.1 ± 3.3 | 8.4 ± 0.44 | 1.5 ± 0.6 | 1.2 | −0.5 | 1.9 | 0.4 | 90 | 47 |
| F123A | | | | | | | | | 79 | nd |

NB = No Detectable Binding;
nd = not determined for data quality control;
*= potential conformational destabilization or indirect contacts

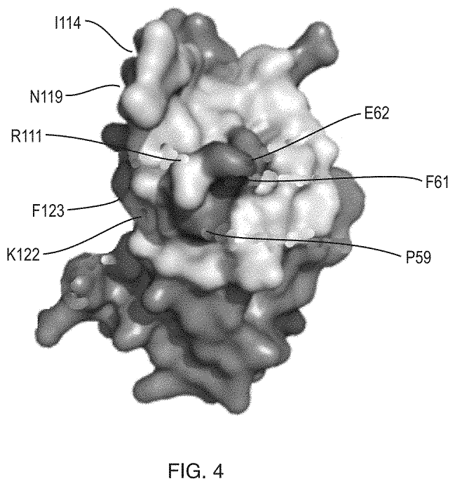

M6903 showed a decrease or loss of binding for the TIM-3 single point mutants P59A, F61A, E62A, I114A, N119A, and K122A (see TABLE 7). Residues P59A, F61A, E62A, I114A, N119A, and K122A reside on the face of one beta sheet of the immunoglobulin fold as shown with the model (see FIG. 4) and are present in the CC′ and FG loops of human TIM-3, loops which have been shown to be involved in Ptd-Ser binding. Contact with the sidechain of Ile-114 by M6903 is not evident; the moderate deleterious effects due to mutation are explained as local destabilization of the loop region. The closest cross-interface contacts for Lys-122 are 4.7A and occur with backbone carbonyls of the antibody. Water-bridging interactions are possible at this distance but could not be observed given the resolution of the crystal structure. The deleterious effects of the K122A mutation may be explained if the gap is bridged via water bridging.

TIM-3 mutants R111 and F123 showed low stability as assessed by SEC, FMTU, and any reduced binding observed for R111 and F123 mutants likely due to destabilization of the protein and not critical interactions with the antibody. Therefore, TABLE 7 indicates hotspot residues for binding of M6903 to include P59A, F61A, and E62A (see also FIG. 4).

The experiment was repeated using known antibodies ABTTM3-h03 ABTTM3-mAB 15, and 27.12E12. Results are shown in TABLE 7 and TABLE 8. For known antibody mAb h03, residues P59A, 1114A, M118A, and K122A are identified as residues in the binding interface given the effect on binding. In particular, K122 and F123 are shown as hotspots for mAb h03. These positions are in the reported binding footprint for mAb h03 (US20150218274A1, hum21 is Fab form of h03). Accordingly, while some mutant hu TIM-3 proteins resulted in loss of binding to M6903 and ABTIM3-h03, other huTIM-3 mutants resulted in loss of binding only to M6903, suggesting that the two antibodies have partially overlapping but distinct epitopes.

The other known antibodies, 27.12E12 and mab15, do not have hotspots revealed among this set of TIM-3 variant proteins despite competition observed in epitope binning experiments, suggesting that M6903 and ABTIM-3-mab15 have non-overlapping epitopes.

TABLE 8
Mutational scanning identifies hotspot residues in M6903 epitope

| | | 3903B11-IgG1 (non- | | AB TIM3- |
|---|---|---|---|---|
| | M6903 | competitive control) | AB TIM3-h03 | mab15 |
| Ligand | KD (nM) | | | |
| hu TIM3 | 5.7 | 31 | 0.3 | 0.8 |
| P59A | ND† | 23 | 0.9*° | 0.6 |
| V60A | 3.7 | 23 | 0.4 | 0.5 |
| F61A | ND† | 27 | 0.6 | 0.8 |
| E62A | 88‡ | 19 | 0.4 | 0.5 |
| R111A | ND*† | 28 | 4.1*‡ | 3.0*° |
| R111E | ND*† | 99*° | 7.2*‡ | 2.4*° |
| I114A | 29*‡ | 27 | 0.7 | 0.6 |
| M118A | 10 | 30 | 0.7 | 1.1 |
| N119A | 17*° | 18 | 0.7 | 1.1 |
| K122A | 47*‡ | 36 | 8.4‡ | 1.8 |
| F123A | ND*† | 56 | 27† | 3.4° |

ΔΔGₘᵤₜ > 2†; >1‡; >0.5° kcal/mol
ND = No binding detectable
* = potential conformational destabilization or indirect contacts

### Example 5—Pharmacology Studies for Anti-TIM-3 Antibodies

The following studies refer to the anti-TIM3 antibody M6903. M6903 contains the light and heavy chain variable regions of 3903E11 (VL1.3,VH1.2) in an IgG2h(FN-AQ,322A)-delK background (anti-TIM3-3903E11(VL1.3,VH1.2)-IgG2h(FN-AQ,322A)-delK). The light and heavy chains of M6903 correspond to SEQ ID NO: 21 and SEQ ID NO: 22, respectively.

5.1 Target Occupancy of anti-TIM-3

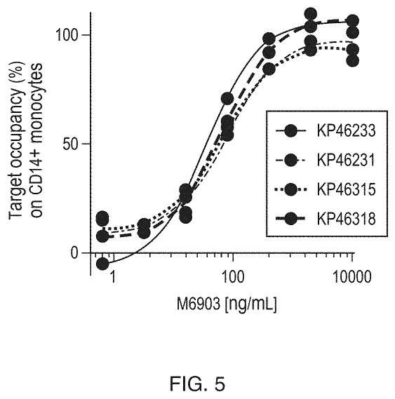

The ability of M6903 to bind to TIM-3 was demonstrated using anti-TIM-3 (A16-019-1), which is identical to M6903, but produced in Expi293F, not CHOK1SV, cells. The target occupancy of anti-TIM-3 (A16-019-1) on CD14+ monocytes was measured via flow cytometry using human whole blood samples. The samples were incubated with serial dilutions of anti-TIM-3 (A16-019-1) followed by anti-TIM-3(2E2)-APC, which has been shown to compete with anti-TIM-3 (A16-019-1) in binding to TIM-3 on CD14+ monocytes. As expected, target occupancy % increased with increasing concentrations of anti-TIM-3 (A16-019-1), and the average EC50 across all 10 donors was 111.1±85.6 ng/ml (see FIG. 5). The highest doses were shown to saturate.

5.2 M6903 Efficiently Blocked the Interaction of rhTIM-3 and PtdSer on Apoptotic Jurkat Cells.

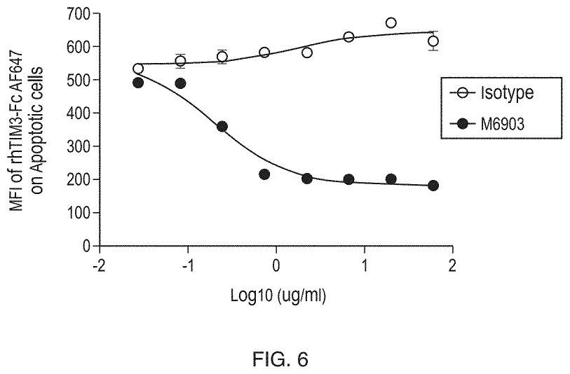

The ability of M6903 to block the interaction of TIM-3 with one if its ligands, PtdSer, was determined by a flow cytometry-based binding assay. Apoptotic Jurkat cells were used as the source for PtdSer, as the induction of apoptosis led to PtdSer exposure on the cell membrane of these cells. Specifically, prior to flow cytometry analysis, apoptosis was induced in Jurkat cells via treatment with Staurosporine (2 μg/mL, 18 hrs), leading to surface expression of a TIM-3 ligand, PtdSer. Binding of rhTIM-3-Fc PtdSer on the surface of apoptotic Jurkat cells was evaluated via flow cytometry by measuring the mean fluorescence intensity (MFI) of rhTIM-3-Fc AF647 after pre-incubation with serial dilutions of M6903 or an anti-HEL IgG2h isotype control. Pre-incubation of rhTIM-3 AF647 with M6903 led to reduced binding of TIM-3-Fc to apoptotic Jurkat cells, whereas pre-incubation with an isotype control had no effect on rhTIM-3-Fc binding (see FIG. 6). Therefore, M6903 was able to efficiently block the interaction between TIM-3 and PtdSer in a dose-dependent manner, with an IC50 of 4.438±3.115 nM (0.666±0.467 μg/mL). A nonlinear fit line was applied to the graph using a Sigmoid dose-response equation. It is hypothesized that this blockade of TIM-3/PtdSer interactions might lead to suppression of the inhibitory TIM-3 signaling and, as a result, enhanced immune cell activation.

5.3 Effect of M16903 on T Cell Recall Response and Activation as Monotherapy or in Combination with Avelumab or Bintrafusp Alfa

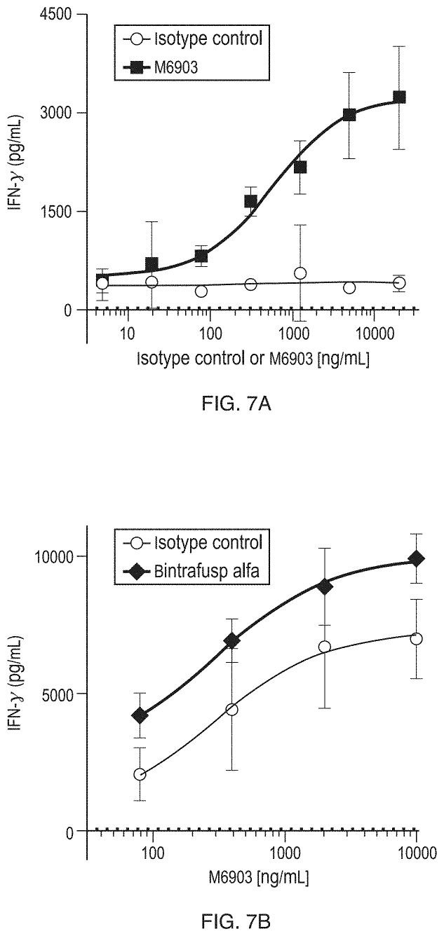

M6903 treatment increased IFN-γ production from human PBMCs that were activated by exposure to CEF antigens, which specifically elicits CEF antigen-specific T cell recall responses in the PBMCs from the donors who were previously infected with CEF. PBMCs were treated with 40 μg/ml CEF viral peptide pool for (A) 6 days or (B) 4 days in the presence of serial dilutions of M6903. As shown FIG. 7A, M6903 dose-dependently enhanced T cell activation compared to isotype control in a CEF assay as measured by IFN-γ production using a human IFN-γ ELISA kit, with an EC50 of 1±1.3 μg/mL, calculated from multiple experiments. Non-linear regression analysis was performed and mean and SD are presented.

As shown in FIG. 7B, serial dilutions of M6903 were combined with either 10 μg/mL isotype control or bintrafusp alfa. The production of IFN-γ was further enhanced in the presence of the combination of M6903 and bintrafusp alfa, suggesting that the combination might lead to further enhancement in T cell activation. Mean and SD are presented (p<0.05) in FIG. 7B.

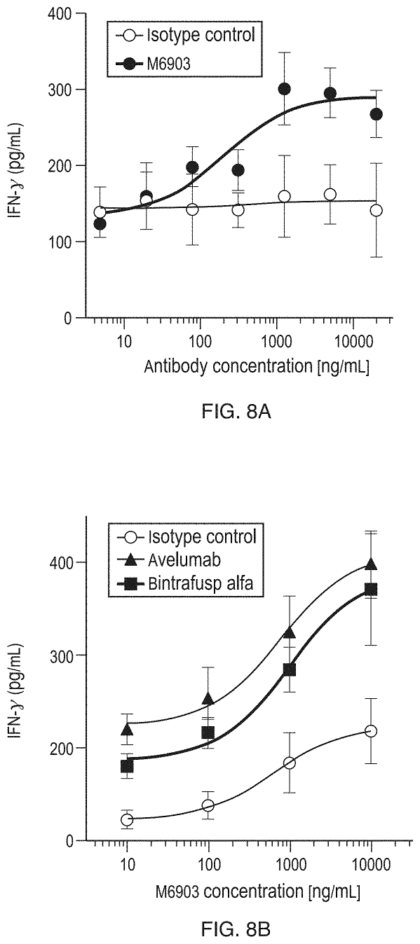

Irradiated Daudi tumor cells were co-cultured with human T cells for 7 days using IL-2 to induce allogenic reactive T cell expansion. The T cells were then harvested and co-cultured with freshly irradiated Daudi cells and treated with M6903 antibody or isotype control for 2 days. T cell activation was measured by an IFN-γ ELISA, and M6903 was shown to dose-dependently enhance IFN-γ production in these cells compared to the isotype control, with an EC50=116±117 ng/mL (see FIG. 8). The addition of avelumab or bintrafusp alfa further enhanced the effect of M6903 on T cell activation (see FIG. 8).

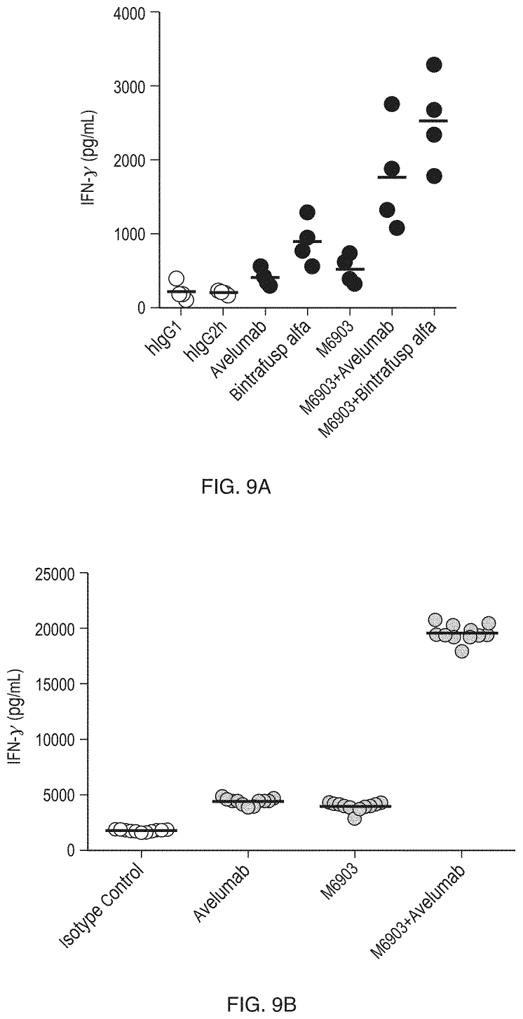

M6903 treatment increased IFN-γ production in human PBMCs that were activated by exposure to superantigen SEB, which activates CD4⁺ T cells non-specifically via cross-linking T cell receptor (TCR) and MHC class II molecules. M6903 (10 μg/mL) was incubated with 100 ng/mL SEB either alone or in combination with avelumab or bintrafusp alfa (10μg/mL) for 9 days, and cells were then washed once with medium and re-stimulated with 100 ng/mL SEB and antibody solutions with the same concentrations for an additional 2 days. Human IFN-γ in the supernatant was measured by using a human IFN-γ ELISA kit. M6903 treatment enhanced IFN-γ production (see FIGS. 9A and 9B). When M6903 treatment was combined with avelumab or bintrafusp alfa, the production of IFN-γ was further enhanced (see FIGS. 9A and 9B).

5.4 Dual Blocking of Gal-9/PtdSer is Required to Potentiate T-Cell Activity, Correlating with M6903 Activity

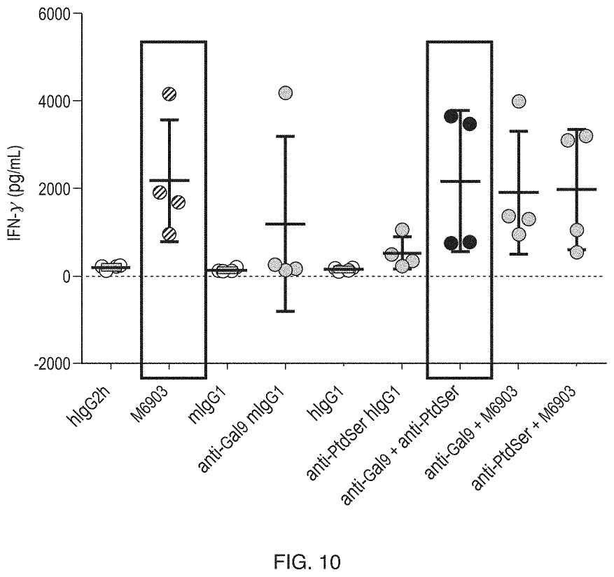

PBMCs were stimulated with 40 μg/ml CEF (Cytomegalovirus, Epstein Barr and Influenza) viral peptide pool (AnaSpec, AS-61036-025) for 4 days in AIM-V medium (Invitrogen #12055-091) with 5% human AB serum (Valley Biomedical, HP1022) in the presence of 10 μg/ml M6903, 10 μg/ml anti-Gal-9 (9M1-3; Biolegend, 348902), or 10 μg/ml anti-PtdSer (bavituximab; Creative Biolabs, TAB-175), or with antibody combinations 10 μg/ml M6903 and 10 μg/ml anti-Gal-9, 10 μg/ml M6903 and 10 μg/ml anti-PtdSer, or 10 μg/ml anti-Gal-9 and 10 μg/ml anti-PtdSer. Proliferation was measured by thymidine incorporation. IFN-γ in culture supernatant was measured by ELISA (R&D Systems, DY285B) and the results are shown in FIG. 10 (representative of at least 3 experiments; p<0.05. As shown, the combination of anti-Gal-9 and anti-PtdSer, but not either antibody alone, exhibited similar activity to M6903 in the CEF assay, suggesting that blocking the binding of both Gal-9 and PtdSer to TIM-3 might be required for anti-TIM-3 activity in this assay. In addition, the combination of M6903 with anti-Gal-9 or anti-PtdSer did not further increase IFNγ production, suggesting that M6903 fully blocked the binding of both Gal-9 and PtdSer to TIM-3.

5.5 Profiling TIM-3 Receptor and Ligand Expression in Normal Human Tissue and Tumors

Expression of TIM-3 and its ligands were then explored using chromogenic IHC and mIF validated assays. TIM-3 expression in normal human tissues was then evaluated using FDA normal tissue microarrays (TMA) representing 35 distinct tissues in the human body. Expression of TIM-3 was observed across most tissues and was specific to immune cells, except in the kidney cortex, where specific TIM-3 expression was also observed on epithelial cells. Highest immune reactivity was observed in immune tissues: spleen, tonsil, and lymph node, as well as in immune-rich organs: lung, placenta, and liver tissues. In immune organs, TIM-3 expression was primarily observed on macrophages (and possibly DCs) but not on lymphocytes (data not shown). TIM-3 expression on lymphocytes was observed only in inflamed tissue (data not shown).

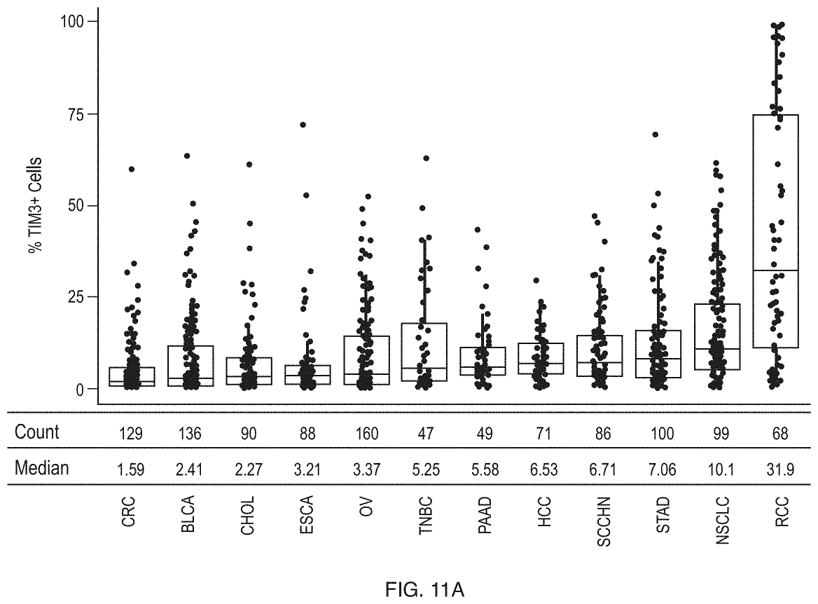

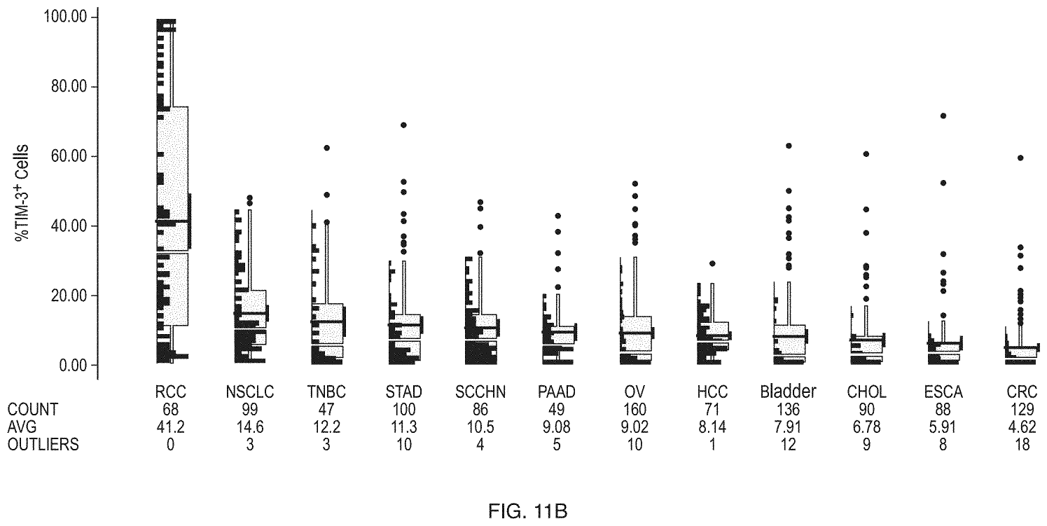

A review of the staining patterns across 15 tumor TMAs, representing 12 different tumor types, showed that TIM-3 expression was observed primarily on infiltrating immune cells across all indications except renal cell carcinoma (RCC). Phenotypically, both T cells and myeloid cells stained positive for TIM-3 (data not shown). Tumor cell expression of TIM-3 was seen only in RCC (data not shown). When the frequency of TIM-3⁺ cells was quantified using digital image analysis staining from these tumor TMAs, RCC showed the highest frequency of TIM-3 positivity (see FIGS. 11A and 11B), potentially due to the expression of TIM-3 on tumor cells in RCC, but not in other tumor types. The data were analyzed by (1) calculating mean expression and plotting the data by ascending median expression (FIG. 11A) and (2) calculating average expression following the removal of outliers and plotting the data by descending median expression (FIG. 11B). Other indications with high TIM-3 levels included NSCLC, stomach adenocarcinoma (STAD), triple negative breast cancer (TNBC) and squamous cell head and neck cancer (SCCHN) (see FIGS. 11A and 11B).

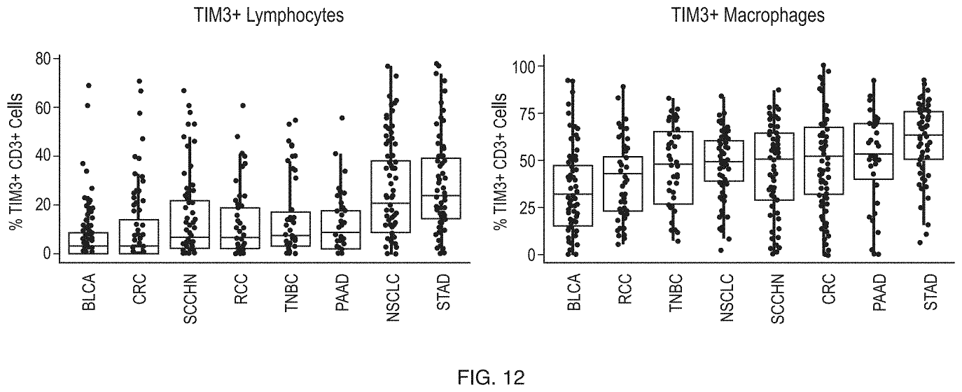

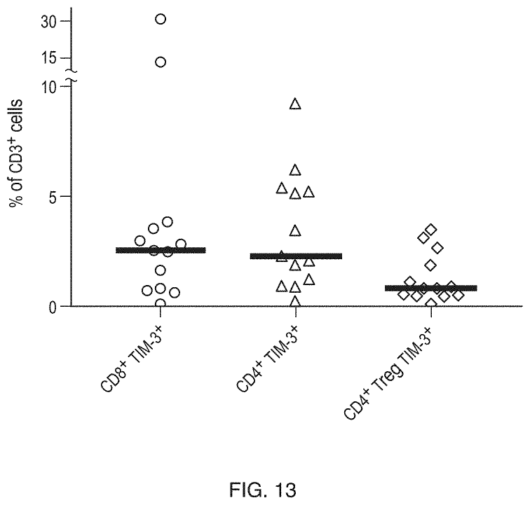

Tumor TMAs were then stained to identify immune cells expressing TIM-3 in the TME using mIF analysis. TIM-3 was found to be expressed on a subset of CD3⁺ lymphocytes and CD68⁺ macrophages. Digital quantitation showed that, while macrophages formed a significant fraction of TIM-3⁺ cells across all indications analyzed, a high frequency of TIM-3⁺ T cells were observed only in NSCLC and STAD tumors (see FIG. 12). These results were confirmed with flow cytometry analysis in a cohort of 13 NSCLC tumor samples; within the live CD3⁺ population, CD8⁺ T cells had the highest median percentage of TIM-3⁺ cells (5.126±2.331%), followed by CD4⁺ effector cells (3.398±0.732%), and CD4⁺ Tregs (1.316±0.310%) (see FIG. 13).

Finally, correlation of TIM-3 expression with ligands, Gal-9, CEACAM-1, and HMGB1, was evaluated both in the TCGA RNASeq data and mIF analyses (see TABLE 9). Pearson correlation of TIM-3 expression with expression of ligands (mRNA and protein), showed that Gal-9 expression was positively correlated across multiple indications. This was not true for CEACAM-1 and HMGB1 expression. Values approaching 1 are the most positively correlated and those approaching -1 are the most negatively correlated, with values near 0 showing little to no correlation.

TABLE 9
Detection of TIM-3 and its ligands using mIF analysis

| | n | % GAL9⁺_area | % CEACAM⁺ | % HMGB⁺ |
|---|---|---|---|---|
| SCLC | 6 | 0.95 | 0.88 | −0.91 |
| TNBC | 46 | 0.8 | −0.05 | 0.29 |
| Bladder | 25 | 0.79 | 0.6 | 0.25 |
| Melanoma | 22 | 0.79 | 0.52 | 0.46 |
| Breast | 46 | 0.68 | 0.7 | 0.42 |
| Endometrial | 28 | 0.59 | 0.43 | 0.53 |
| Lung | 35 | 0.57 | −0.25 | 0.09 |
| Ovarian | 25 | 0.52 | 0.25 | 0.13 |
| SCCHN | 75 | 0.44 | 0.21 | 0.27 |
| NSCLC | 97 | 0.41 | −0.03 | 0.07 |
| H&N | 32 | 0.4 | −0.36 | 0.02 |
| Prostate | 43 | 0.39 | −0.16 | −0.08 |
| Mouth | 17 | 0.36 | 0.09 | 0.44 |
| Kidney | 27 | 0.32 | −0.23 | 0.13 |
| Lymphoma | 43 | 0.32 | 0.17 | −0.21 |
| Gastric | 19 | 0.28 | −0.04 | 0.03 |
| Thyroid | 22 | 0.22 | −0.21 | 0.32 |
| Colon | 24 | −0.07 | −0.42 | −0.16 |

5.6 Explant Platform

Due to the lack of cross-reactivity of human TIM-3 protein with mouse TIM-3 protein, in vivo models are not readily available to interrogate the antitumor activity of M6903. Therefore, to determine whether M6903 had any anti-tumor efficacy, the CANscript™ human tumor microenvironment (TME) platform (developed at MITRA Biotech) was used. The CANscript™ platform is a functional assay that replicates a patient's personal tumor microenvironment, including the immune compartment. Responses to drug treatment applied to pieces of the tumor tissue in vitro are read out using multiple biochemical and phenotypic assays. These tumor responses are integrated by CANscript™ technology's algorithm into a single ‘M’-score that can predict efficacy of the drug.

Using this platform, M6903 was tested in samples from 20 patients with squamous cell carcinoma of the head and neck (SCCHN) either as monotherapy or in combination with bintrafusp alfa. The M-Score predicts treatment outcome based on multiple input parameters for the given tumor specimen. A positive prediction of response correlates to an M-Score greater than 25 (bold numbers in TABLE 10). A negative prediction of response correlates to an M-Score of 25 or lower. There are no M-Scores for the Control treatment as M-Score values are derived from parameters relative to the control untreated samples.

Using M-score as a readout of efficacy, positive predicted response was observed in 3/20 (15%) of tumor samples treated with M6903, 7/20 (35%) of tumor samples treated with bintrafusp alfa, and 9/20 (45%) of tumor samples treated with a combination of M6903 and bintrafusp alfa (see Table 9), suggesting that M6903 has anti-tumor activity which is increased in combination with bintrafusp.

TABLE 10
M-Score analysis for cumulative SCCHN tumors

| | | Bintrafusp | | Bintrafusp |
|---|---|---|---|---|
| S. No | Patient ID | alfa | M6903 | alfa + M6903 |
| 1 | HNS1 | 17 | 19 | 29 |
| 2 | HNS3 | 26 | 26 | 13 |
| 3 | HNS4 | 5 | 17 | 18 |
| 4 | HNS5 | 28 | 9 | 27 |
| 5 | HNS7 | 18 | 10 | 20 |
| 6 | HNS9 | 22 | 11 | 21 |
| 7 | HNS10 | 35 | 18 | 7 |
| 8 | HNS11 | 9 | 17 | 27 |
| 9 | HNS13 | 2 | 11 | 7 |
| 10 | HNS15 | 30 | 22 | 29 |
| 11 | HNS16 | 26 | 16 | 10 |
| 12 | HNS18 | 9 | 13 | 26 |
| 13 | HNS19 | 29 | 21 | 30 |
| 14 | HNS20 | 26 | 32 | 26 |
| 15 | HNS21 | 9 | 8 | 12 |
| 16 | HNS22 | 15 | 22 | 15 |
| 17 | HNS23 | 18 | 20 | 27 |
| 18 | HNS26 | 17 | 13 | 27 |
| 19 | HNS27 | 1 | 1 | 2 |
| 20 | HNS28 | 22 | 27 | 25 |

### Example 6—In Vivo Anti-TIM-3 Antibody Studies

6.1 Animals

Two TIM-3 knock-in mouse models were used. 7-12 weeks old female and male “hu-TIM-3 KI” mice (C57BL/6 background) were obtained from Nanjing Galaxy Biophanna, and 6-8 weeks old female “B-hu-TIM-3 KI” mice (C57BL/6 background) were obtained from Beijing Biocytogen Co., Ltd. Both humanized mouse models were developed by replacing murine extracellular domain of TIM-3 receptor with a human extracellular domain of TIM-3 receptor in mice on the C57BL/6 genetic background. Nanjing Galaxy BioPharma generated hu-TIM-3 KI mice by replacing the extracellular and transmembrane domains of the murine TIM-3 receptor (exon 2-5) with the corresponding domain of the human TIM-3 receptor using Loxp-PGK/Nco-Loxp cassette recombination. Biocytogen developed B-hu-TIM-3 KI mice using CRISPR/Cas9 recombination technology by replacing only the IgV extracellular domain (exon 2) of mouse with the corresponding human domain, which kept the remaining intracellular and cytoplasmic domains of the mouse TIM-3 receptor intact.

6.2 Immune Profile of M6903/Avelumab-Treated MC38 Tumors Grown in huTIM-3 KI Mice

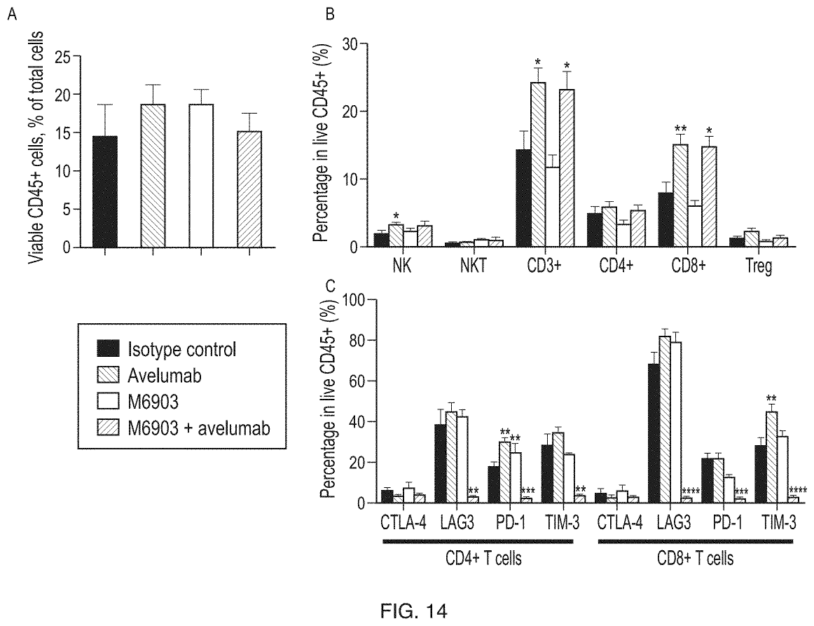

To test the in vivo effects of treatment with M6903 monotherapy or combination with avelumab, MC3 8 colorectal carcinoma cells were subcutaneously implanted into humanized TIM-3 knock-in mice (huTIM-3 KI). The immune cell populations in MC38 tumors established in huTIM-3 KI mice were analyzed via flow cytometry 6 days after the start of treatment with M6903, avelumab, or a combination of the two. Although the percentage of viable CD45+ cells did not differ between the treatment groups (FIG. 14A), the percentage of immune cell populations within the CD45+ population was affected by treatment. Specifically, the percentage of CD3+ cells significantly increased in tumors treated with avelumab (7 mg/kg) monotherapy (P=0.0232) or a combination of M6903 and avelumab (P=0.0445) relative to isotype control (see FIG. 14B). There was also a significant increase of CD8+ T cells with either avelumab monotherapy (P=0.0092) or dual combination therapy (P=0.0127), and an increase in NK cells with avelumab monotherapy (P=0.0172) relative to isotype control (see FIG. 14B). However, there were no significant differences in the percentages of tumor-infiltrating CD3+ leukocytes or CD8+ T cells in tumors treated with combination therapy relative to those treated with avelumab monotherapy.

To evaluate the percentage of T cells within the treated tumors with exhausted phenotypes, the percentage of CD4+ and CD8+ T cells co-expressing the immune checkpoint inhibitors CTLA-4, LAG-3, PD-1, and TIM-3 were analyzed. M6903 monotherapy significantly increased CD4+PD-1+ (P=0.0062) and treatment with avelumab monotherapy increased the percentage of CD4+PD-1+ (P=0.0026) and CD8+TIM-3+ (P=0.0069) cells. However, the combination of M6903 and avelumab had a synergistic effect on these T cell subsets and significantly decreased the percentage of CD4+ cells co-expressing LAG-3 (P=0.0012), PD-1 (P=0.0001), and TIM-3 (P=0.0018), and the percentage of CD8+ cells co-expressing LAG-3 (P<0.0001), PD-1 (P<0.0001), and TIM-3 (P<0.0001) (see FIG. 14C). These results suggest that dual combination of M6903 and avelumab synergizes to decrease exhausted T cells in the tumor microenvironment.

6.3 Anti-Tumor Efficacy of M6903/Avelumab in MC38 Tumor-Bearing B-huTIM-3 KI Mice

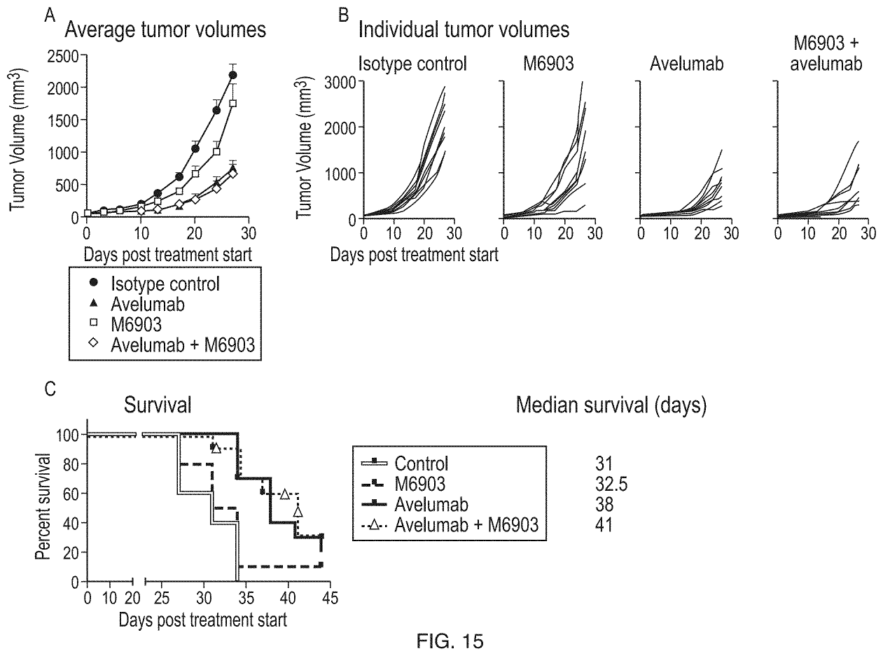

The antitumor efficacy effects of M6903 and avelumab combination therapy were tested in a B-huTIM-3 KI mouse model subcutaneously implanted with MC38 tumors. Mice (N=10/group) were treated with either isotype control (20 mg/kg; i.v; on days 0, 3, 6), avelumab (20 mg/kg; i.v.; on days 0, 3, 6), M6903 (10 mg/kg; i.p.; q3dx7), or the combination of avelumab and M6903. Significant anti-tumor activity was found with avclumab monothcrapy (T/C=34.3%, TGI=67.3%, P<0.0001) or with M6903 monotherapy (T/C=80.4%, TGI=20%, P=0.003 8) relative to isotype control, 27 days after the start of treatment (see FIG. 15A). Combination M6903 and avelumab further enhanced anti-tumor activity (T/C=30.4%, TGI=71.2%) relative to M6903 monotherapy (P<0.0001), though not significantly relative to avelumab monotherapy (see FIG. 15A, B). In addition, combination therapy had a slightly enhanced median survival rate (41 days) relative to isotype control (31 days) and M6903 (32.5 days) and avclumab (38 days) monotherapies (sec FIG. 15C).

6.4 Anti-Tumor Efficacy of M6903/Bintrafusp Alfa in MC38 Tumor-Bearing B-huTIM-3 KI Mice

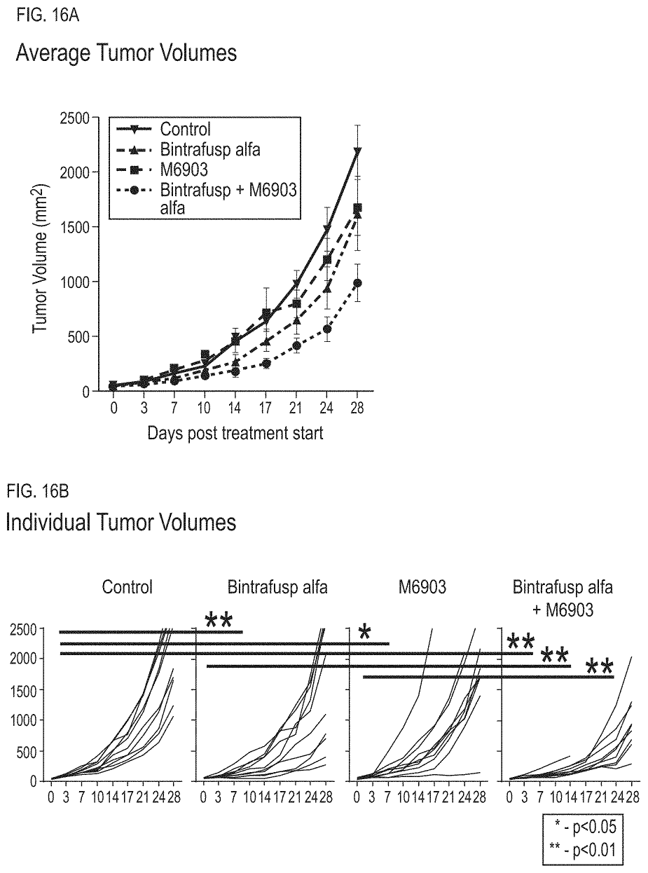

The antitumor efficacy effects of M6903 and bintrafusp combination therapy were tested in a B-huTIM-3 KI mouse model subcutaneously implanted with MC38 tumors. Mice (N=10/group) were treated with either isotype control (20 mg/kg; i.v; on days 0, 3, 6), bintrafusp alfa (24 mg/kg; i.v.; on days 0, 3, 6), M6903 (10 mg/kg; i.p.; q3dx12), or the combination of bintrafusp alfa and M6903. Significant anti-tumor activity was found with bintrafusp monotherapy (TGI=25.7%, P=0.0054)) or with M6903 monotherapy (TGI=18.2%, P=0.0281) relative to isotype control, 28 days after the start of treatment (see FIG. 16A). Combination M6903 and bintrafusp alfa further enhanced anti-tumor activity (TGI=54.6%) relative to M6903 (P=0.0011, day 28) and bintrafusp alfa (P=0.0018, day 28) monotherapies (see FIG. 16A, B). No significant treatment associated body weight loss was observed (data not shown).

### INCORPORATION BY REFERENCE

The entire disclosure of each of the patent and scientific documents referred to herein is incorporated by reference for all purposes.

### EQUIVALENTS

The invention may be embodied in other specific forms without departing from the spirit or essential characteristics thereof The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

### SEQUENCE LISTING

### Anti-TIM3 Antibodies: Optimization

SEQUENCE LISTING:
Anti-TIM3 Antibodies: Optimization

| SEQ ID NO: | |
|---|---|
| | CDRH1 3903E11/M6903 |
| 1 | GFTFSSYA |
| | CDRH2 3903E11/M6903 |
| 2 | ISVSGGST |
| | CDRH3 3903E11/M6903 |
| 3 | AKANWGEEDY |
| | CDRL1 3903E11/M6903 |
| 4 | SSDVGGYNY |
| | CDRL2 3903E11/M6903 |
| 5 | DVS |
| | CDRL3 3903E11/M6903 |
| 6 | SSYADSVV |
| | FR-1 of the heavy chain of antibody 3903E11 family, with X |
| | being any residues selected from the group consisting of Q |
| | (glutamine) and E (glutamic acid) |
| 7 | EVQLVXSGGGLVQPGGSLRLSCAAS |
| | FR-2 of the heavy chain of antibody 3903E11 family, with X |
| | being any residues selected from the group consisting of M |
| | (methionine) and L (leucine) |
| 8 | XSWVRQAPGKGLEWVSA |
| | FR-3 of the heavy chain of antibody 3903E11 family, |
| 9 | YYADSVKGRFTISRDNSKNTLYLQMNSLRAEDTAVYYC |
| | FR-4 of the heavy chain of antibody 3903E11 family |
| 10 | WGQGTLVTVSS |
| | FR-1 of the light chain of antibody 3903E11 family, with X₁ |
| | being any residues selected from the group consisting of S |
| | (serine) and Q (glutamine), X₂ being any residues selected |
| | from the group consisting of Y (Lyrosine) and S (serine) and |
| | X₃ being E (glutamic acid) and A (alanine) |
| 11 | X₁X₂X₃LTQPRSVSGSPGQSVTISCTGT |
| | FR-2 of the light chain of antibody 3903E11 family, with X |
| | being any residues selected from the group consisting of F |
| | (phenylalanine) and Y (tyrosine) |
| 12 | VSWYWHPGKAPKLMIX |
| | FR-3 of the light chain of antibody 3903E11 family |
| 13 | KRPSGVPDRFSGSKSGNTASLTISGLQAEDEADYYC |
| | FR-4 of the light chain of antibody 3903E11 family |
| 14 | FGGGTKVTVL |
| | Light Chain Variant 1 3903E11 (VL1.1)-CL |
| 15 | QSALTQPRSVSGSPGQSVTISCTGTSSDVGGYNYVSWYQQHPGKAPKLMIEDVSKRPSGVPD |
| | RFSGSKSGNTASLTISGLQAEDEADYYCSSYADSVVEGGGTKVTVLGQPKAAPSVTLEPPSS |
| | EELQANKATLVCLISDFYPGAVTVAWKADSSPVKAGVETTTPSKQSNNKYAASSYLSLTPEQ |
| | WKSHKSYSCQVTHEGSTVEKTVAPTECS |
| | Light Chain Variable Region 3903E11 (VL1.1) |
| 52 | QSALTQPRSVSGSPGQSVTISCTGTSSDVGGYNYVSWYQQHPGKAPKLMIEDVSKRPSGVPD |
| | RFSGSKSGNTASLTISGLQAEDEADYYCSSYADSVVEGGGTKVTVL |
| | Heavy Chain Variant 1 3903E11 (VH1.1)-g1 |
| 16 | EVQLVQSGGGLVQPGGSLRLSCAASGFTFSSYALSWVRQAPGKGLEWVSAISVSGGSTYYAD |
| | SVKGRFTISRDNSKNTLYLQMNSLRAEDTAVYYCAKANWGFFDYWGQGTLVTVSSASTKGPS |
| | VFPLAPSSKSTSGGTAALGCLVKDYFPEPVTVSWNSGALTSGVHTFPAVLQSSGLYSLSSVV |
| | TVPSSSLGTOTYICNVNHKPSNTKVDKRVEPKSCDKTHTCPPCPAPELLGGPSVFLEPPKPK |
| | DTLMISRTPEVTCVVVDVSHEDPEVKFNWYVDGVEVHNAKTKPREEQYNSTYRVVSVLTVLH |
| | QDWLNGKEYKCKVSNKALPAPIEKTISKAKGQPREPQVYTLPPSREEMTKNQVSLTCLVKGF |
| | YPSDIAVEWESNGQPENNYKTTPPVLDSDGSFFLYSKLTVDKSRWQQGNVESCSVMHEALHN |
| | HYTQKSLSLSPG |
| | Heavy Chain Variable Region 3903E11 (VH1.1) |
| 53 | EVQLVQSGGGLVQPGGSLRLSCAASGFTESSYALSWVRQAPGKGLEWVSAISVSGGSTYYAD |
| | SVKGRFTISRDNSKNTLYLQMNSLRAEDTAVYYCAKANWGFFDYWGQGTLVTVSS |
| | Light Chain Variant 2 3903E11 (VL1.2)-CL |
| 17 | syeLTQPRSVSGSPGQSVTISCTGTSSDVGGYNYVSWYQQHPGKAPKLMIYDVSKRPSGVPD |
| | RFSGSKSGNTASLTISGLQAEDEADYYCSSYADSVVEGGGTKVTVLGQPKAAPSVTLFPPSS |
| | EELQANKATLVCLISDFYPGAVTVAWKADSSPVKAGVETTTPSKQSNNKYAASSYLSLTPEQ |
| | WKSHKSYSCQVTHEGSTVEKTVAPTECS |
| | Light Chain Variable Region 3903E11 (VL1.2) |
| 54 | syeLTQPRSVSGSPGQSVTISCTGTSSDVGGYNYVSWYQQHPGKAPKLMIYDVSKRPSGVPD |
| | RFSGSKSGNTASLTISGLQAEDEADYYCSSYADSVVFGGGTKVTVL |
| | Heavy Chain Variant 2 3903E11 (VH1.2)-g1 |
| 18 | EVQLVESGGGLVQPGGSLRLSCAASGFTFSSYAMSWVRQAPGKGLEWVSAISVSGGSTYYAD |
| | SVKGRFTISRDNSKNTLYLQMNSLRAEDTAVYYCAKANWGFFDYWGQGTLVTVSSASTKGPS |
| | VFPLAPSSKSTSGGTAALGCLVKDYFPEPVTVSWNSGALTSGVHTFPAVLQSSGLYSLSSVV |
| | TVPSSSLGTQTYICNVNHKPSNTKVDKRVEPKSCDKTHTCPPCPAPELLGGPSVFLEPPKPK |
| | DTLMISRTPEVTCVVVDVSHEDPEVKFNWYVDGVEVHNAKTKPREEQYNSTYRVVSVLTVLH |
| | QDWLNGKEYKCKVSNKALPAPIEKTISKAKGQPREPQVYTLPPSREEMTKNQVSLTCLVKGF |
| | YPSDIAVEWESNGQPENNYKTTPPVLDSDGSFFLYSKLTVDKSRWQQGNVFSCSVMHEALHN |
| | HYTQKSLSLSPG |
| | Heavy Chain Variable Region 3903E11 (VH1.2) |
| 24 | EVOLVESGGGLVOPGGSLRLSCAASGFTESSYAMSWVRQAPGKGLEWVSAISVSGGSTYYAD |
| | SVKGRFTISRDNSKNTLYLQMNSLRAEDTAVYYCAKANWGFFDYWGQGTLVTVSS |
| | Light Chain Variant 3 3903E11 (VL1.3)-CL |
| 19 | QSALTQPRSVSGSPGQSVTISCTGTSSDVGGYNYVSWYQQHPGKAPKLMIEDVSKRPSGVPD |
| | RFSGSKSGNTASLTISGLQAEDEADYYCSSYADSVVEGGGTKVTVLGQPKAAPSVTLFPPSS |
| | EELQANKATLVCLISDFYPGAVTVAWKADSSPVKAGVETTTPSKQSNNKYAASSYLSLTPEQ |
| | WKSHKSYSCQVTHEGSTVEKTVAPTECS |
| | Light Chain Variable Region 3903E11 (VL1.3) |
| 23 | QSALTQPRSVSGSPGQSVTISCTGTSSDVGGYNYVSWYWHPGKAPKLMIEDVSKRPSGVPD |
| | RFSGSKSGNTASLTISGLQAEDEADYYCSSYADSVVFGGGTKVTVL |
| | Heavy Chain Variant 3 3903E11 (VH1.3)-g1 |
| 20 | EVQLVESGGGLVQPGGSLRLSCAASGFTESSIALSWVRQAPGKGLEWVSAISVSGGSTYYAD |
| | SVKGRFTISRDNSKNTLYLQMNSLRAEDTAVYYCAKANWGFFDYWGQGTLVTVSSASTKGPS |
| | VFPLAPSSKSTSGGTAALGCLVKDYFPEPVTVSWNSGALTSGVHTFPAVLQSSGLYSLSSVV |
| | TVPSSSLGTQTYICNVNHKPSNTKVDKRVEPKSCDKTHTCPPCPAPELLGGPSVFLFPPKPK |
| | DTLMISRTPEVTCVVVDVSHEDPEVKFNWYVDGVEVHNAKTKPREEQYNSTYRVVSVLTVLH |
| | QDWLNGKEYKCKVSNKALPAPIEKTISKAKGQPREPQVYTLPPSREEMTKNQVSLTCLVKGF |
| | YRSDIAVEWESNGQPENNYKTTPPVLDSDGSFFLYSKLTVDKSRWQQGNVESCSVMHEALHN |
| | HYTQKSLSLSPG |
| | Heavy Chain Variable Region 3903E11 (VH1.3) |
| 55 | EVQLVESGGGLVQPGGSLRLSCAASGFTFSSYALSWVRQAPGKGLEWVSAISVSGGSTYYAD |
| | SVKGRFTISRDNSKNTLYLQMNSLRAEDTAVYYCAKANWGFFDYWGQGTLVTVSS |

M6903 (anti-TIM3-3903E11(VL1.3, VH1.2)-huIqG2h(FN-AQ,K322A)-delK): Amino Acid

M6903 (anti-TIM3-3903E11(VL1.3, VH1.2)-huIgG2h(FN-AQ, K322A)-delK):
Amino Acid

| SEQ ID NO: | |
|---|---|
| | Light Chain |
| 21 | QSALTQPRSVSGSPGQSVTISCTGTSSDVGGYNYVSWYQQHPGKAPKLMIYDVSKRPSGVPDR |
| | FSGSKSGNTASLTISGLQAEDEADYYCSSYADSVVFGGGTKVTVLGQPKAAPSVTLEPPSSEE |
| | LQANKATLVCLISDFYPGAVTVAWKADSSPVKAGVETTTPSKQSNNKYAASSYLSLTPEQWKS |
| | HKSYSCQVTHEGSTVEKTVAPTECS |
| | Heavy Chain |
| 22 | EVQLVESGGGLVQPGGSLRLSCAASGFTFSSYAMSWVRQAPGKGLEWVSAISVSGGSTYYADS |
| | VKGRFTISRDNSKNTLYLQMNSLRAEDTAVYYCAKANWGFFDYWGQGTLVTVSSASTKGPSVF |
| | PLAPCSRSTSESTAALGCLVKDYFPEPVTVSWNSGALTSGVHTFPAVLQSSGLYSLSSVVTVP |
| | SSNFGTQTYTCNVDMKPSNTKVDKTVEPKSSDKTHTCPPCPAPPVAGPSVFLFPPKPKDTLMI |
| | SRTPEVTCVVVDVSHEDPEVQFNWYVDGVEVHNAKTKPREEQAQSTFRVVSVLTVVHQDWLNG |
| | KEYKCAVSNKGLPAPIEKTISKTKGQPREPQVYTLPPSREEMTKNQVSLTCLVKGFYPSDIAV |
| | EWESNGQPENNYKTTPPMLDSDGSFFLYSKLTVDKSRWQQGNVFSCSVMHEALHNHYTQKSLS |
| | LSPG |
| | Light Chain Variable Region (VL1.3) |
| 23 | QSALTQPRSVSGSPGQSVTISCTGTSSDVGGYNYVSWYQQHPGKAPKLMIYDVSKRPSGVPDR |
| | FSGSKSGNTASLTISGLQAEDEADYYCSSYADSVVFGGGTKVTVL |
| | Heavy Chain Variable Region (VH1.2) |
| 24 | EVQLVESGGGLVQPGGSLRLSCAASGFTFSSYAMSWVRQAPGKGLEWVSAISVSGGSTYYADS |
| | VKGRFTISRDNSKNTLYLQMNSLRAEDTAVYYCAKANWGFFDYWGQGTLVTVSS |
| | Light Chain Constant Region |
| 25 | GQPKAAPSVTLFPPSSEELQANKATLVCLISDFYPGAVTVAWKADSSPVKAGVETTTPSKQSN |
| | NKYAASSYLSLTPEQWKSHKSYSCQVTHEGSTVEKTVAPTECS |
| | Heavy Chain Constant Region |
| 26 | ASTKGPSVFPLAPCSRSTSESTAALGCLVKDYFPEPVTVSWNSGALTSGVHTFPAVLQSSGLY |
| | SLSSVVTVPSSNFGTQTYTCNVDHKPSNTKVDKTVEPKSSDKTHTCPPCPAPPVAGPSVFLFP |
| | PKPKDTLMISRTPEVTCVVVDVSHEDPEVQFNWYVDGVEVHNAKTKPREEQAQSTFRVVSVLT |
| | VVHQDWLNGKEYKCAVSNKGLPAPIEKTISKTKGQPREPQVYTLPPSREEMTKNQVSLTCLVK |
| | GFYPSDIAVEWESNGQPENNYKTTPPMLDSDGSFFLYSKLTVDKSRWQQGNVFSCSVMHEALH |
| | NHYTQKSLSLSPG |

M6903 (anti-TIM3-3903E11(VL1.3, VH1.2)-huIgG2h (FN-AQ , K322A) -delK) : Nucleotide

M6903 (anti-TIM3-3903E11(VL1.3, VH1.2)-huIgG2h(FN-AQ,
K322A)-delK): Nucleotide

| | |
|---|---|
| | Light Chain Variable |
| 27 | CAGAGCGCCCTGACACAGCCTCGCTCAGTGTCCGGGTCTCCTGGACAGTCAGTCACCATCTCC |
| | TGCACTGGAACCAGCAGTGATGTTGGTGGTTATAACTATGTCTCCTGGTACCAACAGCACCCA |
| | GGCAAAGCCCCCAAACTCATGATTTACGATGTCAGTAAGCGGCCCTCAGGGGTCCCTGATCGC |
| | TTCTCTGGCTCCAAGTCTGGCAACACGGCCTCCCTGACCATCTCTGGGCTCCAGGCTGAGGAT |
| | GAGGCTGATTATTACTGCTCCTCATATGCAGACAGCGTGGTATTCGGCGGAGGGACCAAGGTG |
| | ACCGTCCTAGG |
| | Heavy Chain Variable |
| 28 | GAGGTGCAGCTGGTGGAGTCTGGGGGAGGCTTGGTACAGCCTGGGGGGTCCCTGAGACTCTCC |
| | TGTGCAGCCTCTGGATTCACCTTTAGCAGCTATGCCATGAGCTGGGTCCGCCAGGCTCCAGGG |
| | AAGGGGCTGGAGTGGGTCTCAGCTATTAGTGTTAGTGGTGGTAGCACATACTACGCAGACTCC |
| | GTGAAGGGCCGATTCACCATCTCCAGAGACAATTCCAAGAACACGCTGTATCTGCAAATGAAC |
| | AGCCTGAGAGCCGAGGACACGGCCGTATATTACTGTGCGAAAGCCAACTGGGGGTTCTTTGAC |
| | TACTGGGGCCAGGGAACCCTGGTCACTGTCTCTTCA |
| | Light Chain Constant |
| 29 | GGACAGCCCAAGGCTGCCCCCTCGGTCACTCTGTTCCCGCCCTCCTCTGAGGAGCTTCAAGCC |
| | AACAAGGCCACACTGGTGTGTCTCATAAGTGACTTCTACCCGGGAGCCGTGACAGTGGCCTGG |
| | AAGGCAGATAGCAGCCCCGTCAAGGCGGGAGTGGAGACCACCACACCCTCCAAACAAAGCAAC |
| | AACAAGTACGCGGCCAGCAGCTACCTGAGCCTGACGCCTGAGCAGTGGAAGTCCCACAAAAGC |
| | TACAGCTGCCAGGTCACGCATGAAGGGAGCACCGTGGAGAAGACAGTGGCCCCTACAGAATGT |
| | TCA |
| | Heavy Chain Constant |
| 30 | GCTAGCACCAAGGGCCCATCGGTCTTCCCCCTGGCGCCCTGCTCCAGGAGCACCTCCGAGAGC |
| | ACAGCGGCCCTGGGCTGCCTGGTCAAGGACTACTTCCCCGAACCGGTGACGGTGTCGTGGAAC |
| | TCAGGCGCTCTGACCAGCGGCGTGCACACCTTCCCAGCTGTCCTACAGTCCTCAGGACTCTAC |
| | TCCCTCAGCAGCGTGGTGACCGTGCCCTCCAGCAACTTCGGCACCCAGACCTACACCTGCAAC |
| | GTAGATCACAAGCCCAGCAACACCAAGGTGGACAAGACAGTTGAGCCCAAATCTTCTGACAAA |
| | ACTCACACATGCCCACCGTGCCCAGCACCACCTGTGGCAGGACCGTCAGTCTTCCTCTTCCCC |
| | CCAAAACCCAAGGACACCCTCATGATCTCCCGGACCCCTGAGGTCACGTGCGTGGTGGTGGAC |
| | GTGAGCCACGAAGACCCCGAGGTCCAGTTCAACTGGTACGTGGACGGCGTGGAGGTGCATAAT |
| | GCCAAGACAAAGCCACGGGAGGAGCAGGCCCAGAGCACGTTCCGTGTGGTCAGCGTCCTCACC |
| | GTTGTGCACCAGGACTGGCTGAACGGCAAGGAGTACAAGTGCGCTGTCTCCAACAAAGGCCTC |
| | CCAGCCCCCATCGAGAAAACCATCTCCAAAACCAAAGGGCAGCCCCGAGAACCACAGGTGTAC |
| | ACCCTGCCCCCATCACGGGAGGAGATGACCAAGAACCAGGTCAGCCTGACCTGCCTGGTCAAA |
| | GGCTTCTACCCCAGCGACATCGCCGTGGAGTGGGAGAGCAATGGGCAGCCGGAGAACAACTAC |
| | AAGACCACACCTCCCATGCTGGACTCCGACGGCTCCTTCTTCCTCTACAGCAAGCTCACCGTG |
| | GACAAGAGCAGGTGGCAGCAGGGGAACGTCTTCTCATGCTCCGTGATGCATGAGGCTCTGCAC |
| | AACCACTACACACAGAAGAGCCTCTCCCTGTCCCCGGGT |

### Parental Antibody 3903E11 (VL1.0, VH1.0): Amino Acid

Parental Antibody 3903E11 (VL1.0, VH1.0): Amino Acid

| | |
|---|---|
| | Light Chain |
| 31 | syeLTQPRSVSGSPGQSVTISCTGTSSDVGGYNYVSWYQQHPGKAPKLMIFDVSKRPSGVPDR |
| | FSGSKSGNTASLTISGLQAEDEADYYCSSYADSVVEGGGTKVTVLGQPKAAPSVTLFPPSSEE |
| | LQANKATLVCLISDFYPGAVTVAWKADSSPVKAGVETTTPSKQSNNKYAASSYLSLTPEQWKS |
| | HKSYSCQVTHEGSTVEKTVAPTECS |
| | Heavy Chain |
| 32 | EVQLVQSGGGLVQPGGSLRLSCAASGFTFSSYAMSWVRQAPGKGLEWVSAISVSGGSTYYADS |
| | VKGRFTISRDNSKNTLYLQMNSLRAEDTAVYYCAKANWGFEDYWGQGTLVTVSSASTKGPSVF |
| | PLAPSSKSTSGGTAALGCLVKDYFPEPVTVSWNSGALTSGVHTFPAVLQSSGLYSLSSVVTVP |
| | SSSLGTQTYICNVNHKPSNTKVDKRVEPKSCDKTHTCPPCPAPELLGGPSVFLFPPKPKDTLM |
| | ISRTPEVTCVVVDVSHEDPEVKENWYVDGVEVHNAKTKPREEQYNSTYRVVSVLTVLHQDWLN |
| | GKEYKCKVSNKALPAPIEKTISKAKGQPREPQVYTLPPSREEMTKNQVSLTCLVKGFYPSDIA |
| | VEWESNGQPENNYKTTPPVLDSDGSFFLYSKLTVDKSRWQQGNVFSCSVMHEALHNHYTQKSL |
| | SLSPG |
| | Light Chain Variable Region (VL1.0) |
| 33 | syeLTQPRSVSGSPGQSVTISCTGTSSDVGGYNYVSWYQQHPGKAPKLMIFDVSKRPSGVPDR |
| | FSGSKSGNTASLTISGLQAEDEADYYCSSYADSVVFGGGTKVTVL |
| | Heavy Chain Variable Region (VH1.0) |
| 34 | EVQLVQSGGGLVQPGGSLRLSCAASGFTFSSYAMSWVRQAPGKGLEWVSAISVSGGSTYYADS |
| | VKGRFTISRDNSKNTLYLQMNSLRAEDTAVYYCAKANWGFFDYWGQGTLVTVSS |
| | Light Chain Constant Region (CL) |
| 35 | GQPKAAPSVTLEPPSSEELQANKATLVCLISDFYPGAVTVAWKADSSPVKAGVETTTPSKQSN |
| | NKYAASSYLSLTPEQWKSHKSYSCQVTHEGSTVEKTVAPTECS |
| | Heavy Chain Constant Region (IgG1m3) |
| 36 | ASTKGPSVFPLAPSSKSTSGGTAALGCLVKDYFPEPVTVSWNSGALTSGVHTFPAVLQSSGLY |
| | SLSSVVTVPSSSLGTQTYICNVNHKPSNTKVDKRVEPKSCDKTHTCPPCPAPELLGGPSVFLF |
| | PPKPKDTLMISRTPEVTCVVVDVSHEDPEVKFNWYVDGVEVHNAKTKPREEQYNSTYRVVSVL |
| | TVLHQDWLNGKEYKCKVSNKALPAPIEKTISKAKGQPREPQVYTLPPSREEMTKNQVSLTCLV |
| | KGFYPSDIAVEWESNGQPENNYKTTPPVLDSDGSFFLYSKLTVDKSRWQQGNVESCSVMHEAL |
| | HNHYTQKSLSLSPG |

### Antibody 3903E11 Hit: Nucleotide

Antibody 3903E11 Hit: Nucleotide

| | |
|---|---|
| | Light Chain Variable Region |
| 37 | tcctatgagCTGACACAGCCTCGCTCAGTGTCCGGGTCTCCTGGACAGTCAGTCACCATCTCC |
| | TGCACTGGAACCAGCAGTGATGTTGGTGGTTATAACTATGTCTCCTGGTACCAACAGCACCCA |
| | GGCAAAGCCCCCAAACTCATGATTTTTGATGTCAGTAAGCGGCCCTCAGGGGTCCCTGATCGC |
| | TTCTCTGGCTCCAAGTCTGGCAACACGGCCTCCCTGACCATCTCTGGGCTCCAGGCTGAGGAT |
| | GAGGCTGATTATTACTGCTCCTCATATGCAGACAGCGTGGTATTCGGCGGAGGGACCAAGGTG |
| | ACCGTCCTA |
| | Heavy Chain Variable Region |
| 38 | GAGGTGCAGCTGGTGCAGTCTGGGGGAGGCTTGGTACAGCCTGGGGGGTCCCTGAGACTCTCC |
| | TGTGCAGCCTCTGGATTCACCTTTAGCAGCTATGCCATGAGCTGGGTCCGCCAGGCTCCAGGG |
| | AAGGGGCTGGAGTGGGTCTCAGCTATTAGTGTTAGTGGTGGTAGCACATACTACGCAGACTCC |
| | GTGAAGGGCCGATTCACCATCTCCAGAGACAATTCCAAGAACACGCTGTATCTGCAAATGAAC |
| | AGCCTGAGAGCCGAGGACACGGCCGTATATTACTGTGCGAAAGCCAACTGGGGGTTCTTTGAC |
| | TACTGGGGCCAGGGAACCCTGGTCACTGTCTCTTCA |
| | Light Chain Constant Region |
| 39 | GGACAGCCCAAGGCTGCCCCCTCGGTCACTCTGTTCCCGCCCTCCTCTGAGGAGCTTCAAGCC |
| | AACAAGGCCACACTGGTGTGTCTCATAAGTGACTTCTACCCGGGAGCCGTGACAGTGGCCTGG |
| | AAGGCAGATAGCAGCCCCGTCAAGGCGGGAGTGGAGACCACCACACCCTCCAAACAAAGCAAC |
| | AACAAGTACGCGGCCAGCAGCTACCTGAGCCTGACGCCTGAGCAGTGGAAGTCCCACAAAAGC |
| | TACAGCTGCCAGGTCACGCATGAAGGGAGCACCGTGGAGAAGACAGTGGCCCCTACAGAATGT |
| | TCA |
| | Heavy Chain Constant Region (IgG1m3) |
| 40 | GCTAGCACCAAGGGCCCATCGGTCTTCCCCCTGGCACCCTCCTCCAAGAGCACCTCTGGGGGC |
| | ACAGCGGCCCTGGGCTGCCTGGTCAAGGACTACTTCCCCGAACCGGTGACGGTGTCGTGGAAC |
| | TCAGGCGCCCTGACCAGCGGCGTGCACACCTTCCCGGCTGTCCTACAGTCCTCAGGACTCTAC |
| | TCCCTCAGCAGCGTGGTGACCGTGCCCTCCAGCAGCTTGGGCACCCAGACCTACATCTGCAAC |
| | GTGAATCACAAGCCCAGCAACACCAAGGTGGACAAGAGAGTTGAGCCCAAATCTTGTGACAAA |
| | ACTCACACATGCCCACCGTGCCCAGCACCTGAACTCCTGGGGGGACCGTCAGTCTTCCTCTTC |
| | CCCCCAAAACCCAAGGACACCCTCATGATCTCCCGGACCCCTGAGGTCACATGCGTGGTGGTG |
| | GACGTGAGCCACGAAGACCCTGAGGTCAAGTTCAACTGGTACGTGGACGGCGTGGAGGTGCAT |
| | AATGCCAAGACAAAGCCGCGGGAGGAGCAGTACAACAGCACGTACCGTGTGGTCAGCGTCCTC |
| | ACCGTCCTGCACCAGGACTGGCTGAATGGCAAGGAGTACAAGTGCAAGGTCTCCAACAAAGCC |
| | CTCCCAGCCCCCATCGAGAAAACCATCTCCAAAGCCAAAGGGCAGCCCCGAGAACCACAGGTG |
| | TACACCCTGCCCCCATCACGGGAGGAGATGACCAAGAACCAGGTCAGCCTGACCTGCCTGGTC |
| | AAAGGCTTCTATCCCAGCGACATCGCCGTGGAGTGGGAGAGCAATGGGCAGCCGGAGAACAAC |
| | TACAAGACCACGCCTCCCGTGCTGGACTCCGACGGCTCCTTCTTCCTCTATAGCAAGCTCACC |
| | GTGGACAAGAGCAGGTGGCAGCAGGGGAACGTCTTCTCATGCTCCGTGATGCATGAGGCTCTG |
| | CACAACCACTACACGCAGAAGAGCCTCTCCCTGTCCCCGGGT |

TIM3 Sequences (and others)

TIM3 Sequences (and others)

SEQ ID NO: 41: human TIM-3 extracellular domain (amino acid sequence,
NP_116171)
SEVEYRAEVGQNAYLPCFYTPAAPGNLVPVCWGKGACPVFECGNVVLRTDERDVNYWTSRYWLNGDFRKGDVSLTIE
NVTLADSGIYCCRIQIPGIMNDEKENLKLVIKPAKVTPAPTRQRDFTAAFPRMLTTRGHGPAETQTLGSLPDINLTQ
ISTLANELRDSRLANDLRDSGATIRIG
SEQ ID NO: 42: cyno TIM-3 extracellular domain (amino acid sequence,
XP_005558438)
SEVEYIAEVGQNAYLPCSYTPAPPGNLVPVCWGKGACPVFDCSNVVLRTDNRDVNDRTSGRYWLKGDFHKGDVSLTI
ENVTLADSGVYCCRIQIPGIMNDEKHNVKLVVIKPAKVTPAPTLQRDLTSAFPRMLTTGEHGPAETQTPGSLPDVNL
TQIFTLTNELRDSGATIRTA
SEQ ID NO: 43: human TIM-3 ECD with His tag (amino acid sequence, Novoprotein
Cat# C356)
SEVEYRAEVGQNAYLPCFYTPAAPGNLVPVCWGKGACPVFECGNVVLRTDERDVNYWTSRYWLNGDFRKGDVSLTIE
NVTLADSGIYCCRIQIPGIMNDEKFNLKLVIKPAKVTPAPTLQRDFTAAFPRMLTTRGHGPAETQTLGSLPDINLTQ
ISTLANELRDSRLANDLRDSGATIRVDHHHHHH
SEQ ID NO: 44: human TIM-3 ECD with His tag (amino acid sequence, Novoprotein
Cat# CD71)
SEVEYRAEVGQNAYLPCFYTPAAPGNLVPVCWGKGACPVFECGNVVLRTDERDVNYWTSRYWLNG
DERKGDVSLTIENVTLADSGIYCCRIQIPGIMNDEKFNLKLVIKPAKVTPAPTLQRDFTAAFPRM
LTTRGHGPAETQTLGSLPDINLTQISTLANELRDSRLANDLRDSGATIRVDDIEGRMDEPKSCDK
THTCPPCPAPELLGGPSVFLFPPKPKDTLMISRTPEVTCVVVDVSHEDPEVKFNWYVDGVEVHNA
KTKPREEQYNSTYRVVSVLTVLHQDWLNGKEYKCKVSNKALPAPIEKTISKAKGQPREPQVYTLP
PSREEMTKNQVSLTCLVKGFYPSDIAVEWESNGQPENNYKTTPPVLDSDGSFFLYSKLTVDKSRW
QQGNVFSCSVMHEALHNHYTQKSLSLSPGKHHHHHH
SEQ ID NO: 45: marmoset TIM-3 ECD (amino acid sequence, Novoprotein Cat#
CM64)
EEYIVEVGQNAYLPCFYTLDTPGNLVPVCWGKGACPVFECGDVVLRTDERDVSYRTSSRYWLNGDFHKGNVTLAIGN
VTLEDSGIYCCRVQIPGIMNDKKFNLKLVIKPAKVTPAPTLPRDSTPAFPRMLTTEDHGPAETQTLEILHDKNLTQL
STLANELQDAGTTIRIHHHHHH
SEQ ID NO: 46: mouse TIM-3 extracellular domain (amino acid sequence,
NP_599011)
RSLENAYVFEVGKNAYLPCSYTLSTPGALVPMCWGKGFCPWSQCTNELLRTDERNVTYQKSSRYQLKGDLNKGDVSL
IIKNVTLDDHGTYCCRIQFPGLMNDKKLELKLDIKAAKVTPAQTAHGDSTTASPRTLTTERNGSETQTLVTLHNNNG
TKISTWADEIKDSGETIRTA
SEQ ID NO: 47: HC of 3903E11 Fab fragment for crystallization
EVQLVESGGGLVQPGGSLRLSCAASGFTFSSYAMSWVRQAPGKGLEWVSAISVSGGSTYYADSVKGRFTISRDNSKN
TLYLQMNSLRAEDTAVYYCAKANWGFFDYWGQGTLVTVSSASTKGPSVFPLAPSSKSTSGGTAALGCLVKDYFPEPV
TVSWNSGALTSGVHTFPAVLQSSGLYSLSSVVTVPSSSLGTQTYICNVNHKPSNTKVDKKVEPKSCAAAHHHHHH
SEQ ID NO: 48: LC of 3903E11 Fab fragment for crystallization
SYELTQPRSVSGSPGQSVTISCTGTSSDVGGYNYVSWYQQHPGKAPKLMIFDVSKRPSGVPDRFSGSKSGNTASLTI
SGLQAEDEADYYCSSYADSVVEGGGTKVTVLGQPKAAPSVTLFPPSSEELQANKATLVCLISDFYPGAVTVAWKADS
SPVKAGVETTTPSKQSNNKYAASSYLSLTPEQWKSHKSYSCQVTHEGSTVEKTVAPTECS
SEQ ID NO: 49: Human TIM-3 ECD (expressed in e. coli for crystallography)
MSEVEYRAEVGQNAYLPCFYTPAAPGNLVPVCWGKGACPVFECGNVVLRTDERDVNYWTSRYWLNGDFRKGDVSLTI
ENVTLADSGIYCCRIQIPGIMNDEKFNLKLVIK
SEQ ID NO: 50: Nucleotide sequence for Human TIM-3 ECD (expressed in e. coli
for crystallography)
ATGAGCGAGGTGGAATATCGGGCCGAAGTGGGCCAGAACGCCTACCTGCCTTGCTTCTACACACCAGCCGCCCCTGG
CAACCTGGTGCCTGTGTGTTGGGGAAAGGGCGCCTGCCCTGTGTTCGAGTGCGGCAACGTGGTGCTGAGAACCGACG
AGCGGGACGTGAACTACTGGACCAGCCGGTACTGGCTGAACGGCGACTTCAGAAAGGGCGACGTGTCCCTGACCATC
GAGAACGTGACCCTGGCCGACAGCGGCATCTACTGCTGCAGAATCCAGATCCCCGGCATCATGAACGACGAGAAGTT
CAACCTGAAGCTCGTGATCAAGTAA
SEQ ID NO: 51 Human TIM-3 Isoform 1 (Uniprot Code Q8TDQ0-1)
MFSHLPFDCVLLLLLLLLTRSSEVEYRAEVGQNAYLPCFYTPAAPGNLVPVCWGKGACPVFECGNVVLRTDERDVNY
WTSRYWLNGDFRKGDVSLTIENVTLADSGIYCCRIQIPGIMNDEKFNLKLVIKPAKVTPAPTRQRDETAAFPRMLTT
RGHGPAETQTLGSLPDINLTQISTLANELRDSRLANDLRDSGATIRIGIYIGAGICAGLALALIFGALIFKWYSHSK
EKTQNLSLTSLANLPPSGLANAVAEGTRSEENIYTIEENVYEVEEPNEYYCYVSSRQQPSQPLGCRFAMP

1. An isolated antibody that binds human TIM-3 comprising
(i) an immunoglobulin heavy chain variable region comprising a CDR_{H1} comprising the amino acid sequence of SEQ ID NO: 1, a CDR_{H2} comprising the amino acid sequence of SEQ ID NO: 2, and a CDR_{H3} comprising the amino acid sequence of SEQ ID NO: 3; and
(ii) an immunoglobulin light chain variable region comprising a CDR_{L1} comprising the amino acid sequence of SEQ ID NO: 4, a CDR_{L2} comprising the amino acid sequence of SEQ ID NO: 5, and a CDR_{L3} comprising the amino acid sequence of SEQ ID NO: 6.

2. An isolated nucleic acid comprising a nucleotide sequence encoding an immunoglobulin heavy chain variable region of claim 1.

3. An isolated nucleic acid comprising a nucleotide sequence encoding an immunoglobulin light chain variable region of claim 1.

4. An expression vector comprising the nucleic acid of claim 2.

5. An expression vector comprising the nucleic acid of claim 3.

6. (canceled)

7. A host cell comprising the expression vector of claim 4.

8. A host cell comprising the expression vector of claim 5.

9.-10. (canceled)

11. A method of producing a polypeptide comprising an immunoglobulin heavy chain variable region, the method comprising:
(a) growing the host cell of claim 7 under conditions so that the host cell expresses the polypeptide comprising the immunoglobulin heavy chain variable region; and
(b) purifying the polypeptide comprising the immunoglobulin heavy chain variable region.

12. (canceled)

13. The isolated antibody of claim 1, comprising an immunoglobulin heavy chain variable region selected from the group consisting of SEQ ID NO: 53, SEQ ID NO: 24, SEQ ID NO: 55, SEQ ID NO: 34, and an immunoglobulin light chain variable region selected from the group consisting of SEQ ID NO: 52, SEQ ID NO: 54, SEQ ID NO: 23 and SEQ ID NO: 33.

14. The isolated antibody of claim 13, comprising an immunoglobulin heavy chain variable region comprising the amino acid sequence of SEQ ID NO: 24, and an immunoglobulin light chain variable region comprising the amino acid sequence of SEQ ID NO: 23.

15.-25. (canceled)

26. The isolated antibody of claim 1, comprising an immunoglobulin heavy chain and an immunoglobulin light chain selected from the group consisting of:
(a) an immunoglobulin heavy chain comprising the amino acid sequence of SEQ ID NO: 22, and an immunoglobulin light chain comprising the amino acid sequence of SEQ ID NO: 21; and
(b) an immunoglobulin heavy chain comprising the amino acid sequence of SEQ ID NO: 32, and an immunoglobulin light chain comprising the amino acid sequence of SEQ ID NO: 31.

27.-37. (canceled)

38. The antibody of claim 1, wherein the antibody has a KD of 9.2 nM or lower, as measured by surface plasmon resonance.

39.-42. (canceled)

43. A method of downregulating at least one exhaustion marker in a tumor microenvironment, the method comprising exposing the tumor microenvironment to an effective amount of the antibody of claim 1 to downregulate at least one exhaustion marker.

44. The method of claim 43, wherein the method further comprises exposing the tumor microenvironment to an effective amount of a second therapeutic agent.

45. The method of claim 44, wherein the second therapeutic agent is an anti-PD-L1 antibody.

46. The method of claim 43, wherein the exhaustion marker is CTLA-4, LAG-3, PD-1, or TIM-3.

47. A method of potentiating T cell activation, the method comprising exposing the T cell to an effective amount of the antibody of claim 1, thereby to potentiate the activation of the T cell.

48. The method of claim 47, wherein the method further comprises exposing the T cell to an effective amount of a second therapeutic agent.

49. A method of inhibiting proliferation of a tumor cell comprising exposing the cell to an effective amount of the antibody of claim 1 to inhibit proliferation of the tumor cell.

50. The method of claim 49, wherein the method further comprises exposing the tumor cell to an effective amount of a second therapeutic agent.

51. A method of inhibiting tumor growth in a mammal, the method comprising exposing the mammal to an effective amount of the antibody of claim 1 to inhibit tumor growth in the mammal.

52. The method of claim 51, wherein the method further comprises exposing the mammal to an effective amount of a second therapeutic agent.

53. A method of treating cancer in a mammal, the method comprising administering an effective amount of the antibody of claim 1 to a mammal in need thereof

54. The method of claim 53, wherein the method further comprises administering an effective amount of a second therapeutic agent to the mammal.

55. The method of claim 53, wherein the cancer is selected from the group consisting of diffuse large B-cell lymphoma, renal cell carcinoma (RCC), non-small cell lung carcinoma (NSCLC), squamous cell carcinoma of the head and neck (SCCHN), triple negative breast cancer (TNBC) or gastric/stomach adenocarcinoma (STAD).

56. The method of claim 53, wherein the mammal is a human.

57.-79. (canceled)
